(19) United States
(12) Patent Application Publication
(10) Pub. No.: US 2017/0087174 A1
(43) Pub. Date: Mar. 30, 2017

(54) COMBINATION THERAPY REGIMEN FOR TREATMENT OF HCV

(71) Applicants: JANSSEN PHARMACEUTICALS, INC., Titusville, NJ (US); ACHILLION PHARMACEUTICALS, INC., New Haven, CT (US)

(72) Inventors: Maria Gloria Beumont, Bievres (FR); Lieve Bijnens, Beerse (BE); Lawrence M. Blatt, S. San Francisco, CA (US); Sushmita Mukherjee Chanda, S.San Francisco, CA (US); John Fry, S.South San Francisco, CA (US); Eugene Jans, Beerse (BE); Thomas Naoki Kakuda, S.San Francisco, CA (US); Sivi Mahadevan, Ravels (BE); Roel Mertens, Beerse (BE); Gaston Rafael Picchio, San Diego, CA (US); Alex Van Dijck, Beerse (BE); Peter Van Remoortere, Princeton, NJ (US); David Apelian, Boonton, NJ (US); Dawei Chen, Guilford, CT (US); Milind Deshphande, Madison, CT (US); James Hui, Apex, NC (US); Avinash Phadke, Branford, CT (US)

(21) Appl. No.: 15/281,050

(22) Filed: Sep. 29, 2016

Related U.S. Application Data
(60) Provisional application No. 62/365,541, filed on Jul. 22, 2016.
(60) Provisional application No. 62/353,708, filed on Jun. 23, 2016.
(60) Provisional application No. 62/299,338, filed on Feb. 24, 2016.
(60) Provisional application No. 62/234,408, filed on Sep. 29, 2015.

(51) Int. Cl. A61K 31/7072; A61K 9/10; A61K 9/00; A61K 31/4709; A61K 31/4184

(52) U.S. Cl. CPC A61K 31/7072; A61K 31/4709; A61K 31/4184; A61K 9/0053; A61K 9/10

(57) ABSTRACT

A composition, including a fixed dose composition, comprising Compound (I), Compound (II), and Compound (III) for the treatment of hepatitis C, and methods of manufacture thereof.

### STATEMENT OF RELATED APPLICATIONS

This application claims the benefit of U.S. Provisional Patent Application Nos. 62/234,408 filed Sep. 29, 2015; 62/299,338 filed Feb. 24, 2016; 62/353,708 filed Jun. 23, 2016 and 62/365,541 filed Jul. 22, 2016. The entirety of these applications are hereby incorporated by reference for all purposes.

### FIELD OF THE INVENTION

The present invention provides a specific combination and regimen of therapeutic compounds for the advantageous treatment of hepatitis C virus infection.

### BACKGROUND OF THE INVENTION

Hepatitis C virus (HCV), a member of the Flaviviridae family of viruses in the hepacivirus genus, is the leading cause of chronic liver disease worldwide. Recent estimates report the global hepatitis C prevalence at around 2.4% with up to 170 million people thought to be chronically infected. Although the development of diagnostics and blood screening has considerably reduced the rate of new infections, HCV remains a global health burden due to its chronic nature and its potential for long-term liver damage. It is now known that HCV has the ability to incorporate into the host's genome.

The hepatitis C virus genome is a small positive-sense single stranded RNA enclosed in a nucleocapsid and lipid envelope. It consists of 9.6 kb ribonucleotides, which encodes a large polypeptide of about 3,000 amino acids (Dymock et al. Antiviral Chemistry & Chemotherapy 2000, 11, 79). Following maturation, this polypeptide is processed into at least ten proteins. NS3/4A serine protease is responsible for the cleavage of the non-structural downstream proteins. NS5A is a zinc-binding proline-rich hydrophilic phosphoprotein which has no apparent enzymatic activity yet has an important function mediating the interaction with other nonstructural viral and cellular proteins. NS5B is an enzyme with polymerase activity that is involved in the synthesis of double-stranded RNA from the single-stranded viral RNA genome, which serves as the template.

NS3/4A serine protease, NS5A and NS5B polymerase are essential for viral replication, and inhibitors are important drug candidates for HCV treatment.

HCV is mainly transmitted by blood contact. Following initial acute infection, a majority of infected individuals develop chronic hepatitis because HCV replicates preferentially in hepatocytes, but is not directly cytopathic. Over decades, a considerable number of infected persons develop fibrosis, at least 30% develop cirrhosis, 1-4% develop hepatocellular carcinoma, and chronic HCV infection is the leading cause for liver transplantation. HCV is responsible for 50-76% of all liver cancer cases and two thirds of all liver transplants in the developed world. This and the number of patients involved has made HCV the focus of considerable medical research.

There are six major HCV genotypes (1-6) and multiple subtypes (represented by letters). Genotype 1a is predominant in North America, while Genotype 1b is predominant in Europe. The HCV genotype is clinically important in determining potential response to therapy and the required duration of such therapy. Standard therapy (pegylated interferon alpha plus ribavirin (a nucleoside analog)) is only effective in 50-60% of patients and is associated with significant side effects.

Due to the number of people infected with HCV and the virus' high mutation rate, there is a pressing need for efficacious new treatments.

A goal of HCV therapy is to provide efficacious, interferon-free treatment for the long term clearance of HCV, which is often pursued through a combination of active compounds. Further goals are potent antiviral activities, high genetic barriers to resistance, broad genotypic coverage, minimal side effects, and a favorable safety profile.

The “SVR” of an HCV regimen refers to the sustained virological response, wherein a “response” means an HCV RNA level of less than the lower limit of quantitation (LLOQ). A “SVRn” refers to an SVR of up to about n weeks after termination of the relevant treatment regime. It is a goal of HCV therapy to achieve a cure, which is currently defined as an SVR of at least 12 weeks (“SVR₁₂”), i.e., evidence that the patient has a sustained HCV level that is less than LLOQ over a 12 week period after cessation of treatment.

To date, a number of fixed dose drug combinations have been approved for the treatment of HCV. Harvoni® (Gilead Sciences, Inc.) contains the NS5A inhibitor ledipasvir and the NS5B inhibitor sofosbuvir. In clinical studies, 96-99% of patients with HCV genotype 1 who had no prior treatment achieved an SVR12 in approximately 12 weeks of therapy. Technivie™ (AbbVie, Inc.) is a fixed-dose combination containing ombitasvir, an NS5A inhibitor; paritaprevir, an NS3/4A protease inhibitor; and ritonavir, a CYP3A inhibitor. The product is indicated in combination with ribavirin for the treatment of patients with genotype 4 chronic hepatitis C without cirrhosis and the treatment course is 12 weeks. Daklinza™ (daclatasvir, Bristol-Myers Squibb) is a HCV NS5A inhibitor indicated for use with sofosbuvir for the treatment of chronic genotype 3 infection. The duration of therapy is 12 weeks. Zepatier™ (Merck & Co.) has recently been approved for the treatment of chronic HCV genotypes 1 and 4. Zepatier™ is a fixed-dose combination product containing elbasvir, an HCV NS5A inhibitor, and grazoprevir, an HCV NS3/4A protease inhibitor. Zepatier™ is indicated with or without ribavirin; the course of therapy is 12 or 16 weeks. Most recently, the U.S. Food and Drug Administration (FDA) approved Epclusa® to treat adult patients with chronic HCV with or without cirrhosis. Epclusa® (Gilead Sciences, Inc.) is a fixed-dose combination tablet containing sofosbuvir and velpatasvir and is the first approved drug for the treatment of all six major forms of HCV. Epclusa® is approved for use in combination with ribavirin and the course of therapy is 12 weeks.

A number of companies continue to carry out research focused on the discovery of new anti-HCV agents and combinations thereof for the treatment of HCV. U.S. patents focused on anti-HCV agents and combinations thereof include U.S. Pat. Nos. 9,382,218; 9,321,753; 9,249,176; 9,233,974; 9,221,833; 9,211,315; 9,194,873; 9,186,369; 9,180,193; 9,156,823; 9,138,442; 9,133,170; 9,108,999; 9,090,559; 9,079,887; 9,073,943; 9,073,942; 9,056,090; 9,051,340; 9,034,863; 9,029,413; 9,011,938; 8,987,302; 8,945,584; 8,940,718; 8,927,484; 8,921,341; 8,884,030; 8,841,278; 8,822,430; 8,772,022; 8,765,722; 8,742,101; 8,741,946; 8,674,085; 8,673,288; 8,669,234; 8,663,648; 8,618,275; 8,580,252; 8,575,195; 8,575,135; 8,575,118; 8,569,302; 8,524,764; 8,513,298; 8,501,714; 8,404,651; 8,273,341; 8,257,699; 8,197,861; 8,158,677; 8,105,586; 8,093,353; 8,088,368; 7,897,565; 7,871,607; 7,846,431; 7,829,081; 7,829,077; 7,824,851; 7,572,621; and 7,326,536.

Additional U.S. patents include patents assigned to Alios: U.S. Pat. Nos. 9,365,605; 9,346,848; 9,328,119; 9,278,990; 9,249,174; 9,243,022; 9,073,960; 9,012,427; 8,980,865; 8,895,723; 8,877,731; 8,871,737; 8,846,896 and 8,772,474; Achillion U.S. Pat. Nos. 9,273,082; 9,233,136; 9,227,952; 9,133,115; 9,125,904; 9,115,175; 9,085,607; 9,006,423; 8,946,422; 8,835,456; 8,809,313; 8,785,378; 8,614,180; 8,445,430; 8,435,984; 8,183,263; 8,173,636; 8,163,693; 8,138,346; 8,114,888; 8,106,209; 8,088,806; 8,044,204; 7,985,541; 7,906,619; 7,902,365; 7,767,706; 7,741,334; 7,718,671; 7,659,399; 7,476,686; 7,439,374; 7,365,068; 7,199,128; and 7,094,807; Cocrystal Pharma Inc. U.S. Pat. Nos. 9,181,227; 9,173,893; 9,040,479 and 8,771,665; Gilead Sciences U.S. Pat. Nos. 9,353,423; 9,346,841; 9,321,800; 9,296,782; 9,296,777; 9,284,342; 9,238,039; 9,216,996; 9,206,217; 9,161,934; 9,145,441; 9,139,604; 9,090,653; 9,090,642; 9,085,573; 9,062,092; 9,056,860; 9,045,520; 9,045,462; 9,029,534; 8,980,878; 8,969,588; 8,962,652; 8,957,046; 8,957,045; 8,946,238; 8,933,015; 8,927,741; 8,906,880; 8,889,159; 8,871,785; 8,841,275; 8,815,858; 8,809,330; 8,809,267; 8,809,266; 8,779,141; 8,765,710; 8,759,544; 8,759,510; 8,735,569; 8,735,372; 8,729,089; 8,722,677; 8,716,264; 8,716,263; 8,716,262; 8,697,861; 8,664,386; 8,642,756; 8,637,531; 8,633,309; 8,629,263; 8,618,076; 8,592,397; 8,580,765; 8,569,478; 8,563,530; 8,551,973; 8,536,187; 8,513,186; 8,513,184; 8,492,539; 8,486,938; 8,481,713; 8,476,225; 8,420,597; 8,415,322; 8,338,435; 8,334,270; 8,329,926; 8,329,727; 8,324,179; 8,283,442; 8,263,612; 8,232,278; 8,178,491; 8,173,621; 8,163,718; 8,143,394. Further patents assigned to Idenix, acquired by Merck, include U.S. Pat. Nos. 9,353,100; 9,309,275; 9,296,778; 9,284,307; 9,249,173; 9,243,025; 9,211,300; 9,187,515; 9,187,496, 9,109,001; 8,993,595; 8,951,985; 8,691,788; 8,680,071; 8,637,475; 8,507,460; 8,377,962; 8,362,068; 8,343,937; 8,299,038; 8,193, 372; 8,093,379; 7,951,789; 7,932,240; 7,902,202; 7,662,798; 7,635,689; 7,625,875; 7,608,600; 7,608,597; 7,582,618; 7,547,704; 7,456,155; 7,384,924; 7,365,057; 7,192,936; 7,169,766; 7,163,929; 7,157,441; 7,148,206; 7,138,376; 7,105,493; 6,914,054 and 6,812,219. Patents assigned to Merck include U.S. Pat. Nos. 9,364,482; 9,339,541; 9,328,138; 9,265,773; 9,254,292; 9,243,002; 9,242,998; 9,242,988; 9,242,917; 9,238,604; 9,156,872; 9,150,603; 9,139,569; 9,120,818; 9,090,661; 9,073,825; 9,061,041; 8,987,195; 8,980,920; 8,927,569; 8,871,759; 8,828,930; 8,772,505; 8,715,638; 8,697,694; 8,637,449; 8,609,635; 8,557,848; 8,546,420; 8,541,434; 8,481,712; 8,470,834; 8,461,107; 8,404,845; 8,377,874; 8,377,873; 8,354,518; 8,309,540; 8,278,322; 8,216,999; 8,148,349; 8,138,164; 8,080,654; 8,071,568; 7,973,040; 7,935,812; 7,915,400; 7,879,815; 7,879,797; 7,632,821; 7,569,374; 7,534,767; 7,470,664 and 7,329,732. Additional U.S. patent application publications include US 2013/0029904 to Boehringer Ingelheim GMBH and US 2014/0113958 to Stella Aps.

Despite the availability of a number of anti-HCV regimens for the treatment of HCV, there remains a need for therapies that can decrease the treatment period, and that have one or a combination of potent antiviral activities, high genetic barriers to resistance, broad genotypic coverage, minimal side effects and favorable safety profiles.

### SUMMARY OF THE INVENTION

The present invention provides a specific combination of drugs using a specific dosage regime that after approximately 8, 7, 6, 5, or even 4 or less weeks of treatment can achieve a sustained virological response. In some embodiments, the treatment regime leads to a sustained virological response of approximately 12, 18, or 24 weeks. In one aspect, the treatment is accomplished with one pill or other dosage form given once a day for the treatment period.

The ability to reach a sustained virological response of at least 12, 18 or 24 weeks using a treatment regime as short as 8, 7, 6, 5, or even 4 or less weeks of treatment is advantageous for the patient because it shortens the duration in which compliance is required, and may minimize the risk of adverse events.

The anti-HCV drugs used in this advantageous combination regime are:
The NS3/4A protease inhibitor Simeprevir (also referred to as Compound (I) or SMV), or a pharmaceutically acceptable hydrate, solvate or salt thereof;
The NS5A inhibitor Odalasvir (also referred to as Compound (II), ODV or ACH-3102), or a pharmaceutically acceptable hydrate, solvate or salt thereof; and
The NS5B polymerase inhibitor of Compound (III), which is isopropyl ((S)-(((2S,3S,4R,5R)-5-(2,4-dioxo-3,4-dihydropyrimidin-1-(2H-yl)-2-fluoro-3,4-dihydroxy-4-methyltetrahydrofuran-2yl)methoxy)(phenoxy)phosphoryl)alaninate, or a pharmaceutically acceptable hydrate, solvate or salt thereof.

Compound (I) (Simeprevir, also referred to as SMV) is:

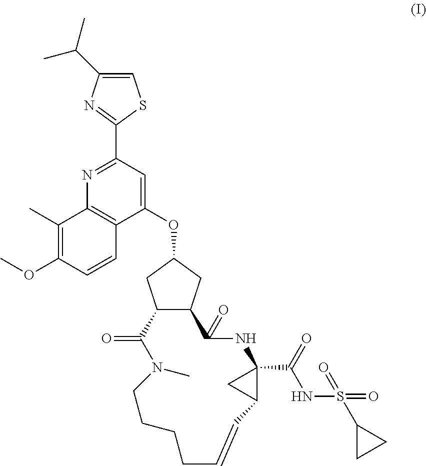 [SMILES: COc1ccc2c(O[C@@H]3C[C@H]4C(=O)N[C@]5(C(=O)NS(=O)(=O)C6CC6)C[C@H]5/C=C/CCCCN(C)C(=O)[C@@H]4C3)cc(-c3nc(C(C)C)cs3)nc2c1C]

Compound (II) (Odalasvir, also known as ACH-3102 and ODV) is:

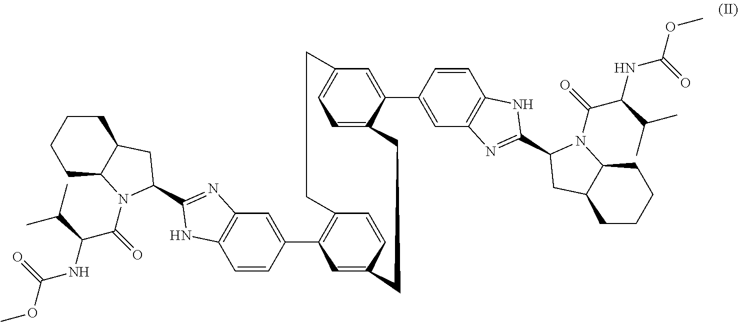

Compound (III) is:

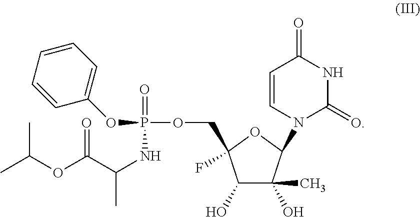 [SMILES: CC(C)OC(=O)C(C)N[P@](=O)(OC[C@@]1(F)O[C@@H](n2ccc(=O)[nH]c2=O)[C@](C)(O)[C@@H]1O)Oc1ccccc1]

Compound (III) is metabolized in vivo to produce the active metabolite A-2 shown below. During the conversion of Compound (III) to A-2, a small amount of A-1 is also produced.

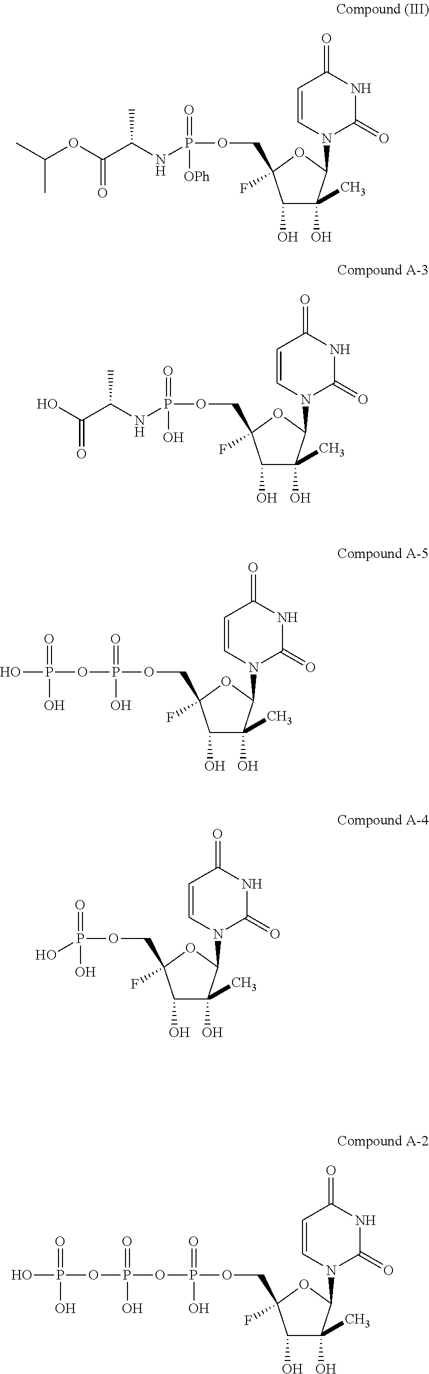 [SMILES: CC(C)OC(=O)[C@H](C)NP(=O)(OC[C@@]1(F)O[C@@H](n2ccc(=O)[nH]c2=O)[C@](C)(O)[C@@H]1O)Oc1ccccc1 ; C[C@H](NP(=O)(O)OC[C@@]1(F)O[C@@H](n2ccc(=O)[nH]c2=O)[C@](C)(O)[C@@H]1O)C(=O)O ; C[C@]1(O)[C@H](n2ccc(=O)[nH]c2=O)O[C@](F)(COP(=O)(O)OP(=O)(O)O)[C@H]1O ; C[C@]1(O)[C@H](n2ccc(=O)[nH]c2=O)O[C@](F)(COP(=O)(O)O)[C@H]1O ; C[C@]1(O)[C@H](n2ccc(=O)[nH]c2=O)O[C@](F)(COP(=O)(O)OP(=O)(O)OP(=O)(O)O)[C@H]1O]

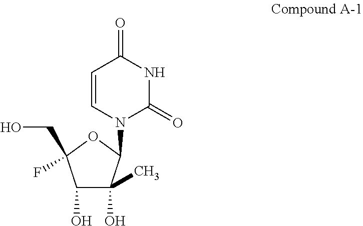 [SMILES: C[C@]1(O)[C@H](n2ccc(=O)[nH]c2=O)O[C@](F)(CO)[C@H]1O]

Compounds (I-III) are provided in an effective amount in combination to treat a patient, typically a human, infected with HCV. In one embodiment, Simeprevir is administered once a day in a dosage of 75 or 100 mg, Odalasvir is administered once a day in a dosage of 25 mg, and Compound (III) is administered in an amount of 800 mg a day. In another embodiment, Odalasvir is provided in the dosage form, or combination of dosage forms, in an amount of 50 mg per day. In another embodiment, Odalasvir is provided in the dosage form, or combination of dosage forms, in an amount of 25 mg per day. In another embodiment, Odalasvir is provided in the dosage form, or combination of dosage forms, in an amount of 12.5 mg per day. In another embodiment, Odalasvir is provided in the dosage form, or combination of dosage forms, in an amount of 10 mg per day. In one aspect, these three drugs are administered in a single dosage form once a day, which may have the benefit of improving treatment compliance. In another embodiment, the three drugs are formulated together into two or more fixed dosage forms, which are administered simultaneously or over the course of the day, for example two or three times a day, as prescribed by a healthcare provider. In yet another embodiment, the three active anti-HCV drugs are provided in separate pills and are administered approximately simultaneously. In another aspect, two of the three drugs are provided in a fixed dose combination and the third is provided in a separate dosage form but administered approximately simultaneously.

In one embodiment, Simeprevir is administered once a day in a dosage of 75 or 100 mg, Odalasvir is administered once a day in a dosage of 12.5 mg, and Compound (III) is administered in a dosage of 800 mg a day. In another embodiment, Simeprevir is administered once a day in a dosage of 75 mg or 100 mg, Odalasvir is administered once a day in a dosage of 20 mg, and Compound (III) is administered in an amount of 800 mg a day. In an additional embodiment, Simeprevir is administered once a day in a dosage of 75 mg or 100 mg, Odalasvir is administered once a day in a dosage of 15 mg, and Compound (III) is administered in an amount of 800 mg a day. In an additional embodiment, Simeprevir is administered once a day in a dosage of 75 mg or 100 mg, Odalasvir is administered once a day in a dosage of 17.5 mg, and Compound (III) is administered in an amount of 800 mg a day. In an additional embodiment, Simeprevir is administered once a day in a dosage of 75 mg or 100 mg, Odalasvir is administered once a day in a dosage of at least 10, 11, 12, 13, 14, 15, 16, 17, 18, 19, 20, 21, 22, 23, 24 or 25 mg (and not more than 20 or 25 mg), wherein each combination is considered and intended to be individually described, and Compound (III) is administered in an amount of 800 mg a day.

In another embodiment, Simeprevir is administered once a day in a dosage of 75 mg or 100 mg, Odalasvir is administered once a day in a dosage of 7.5, 10, 12.5, 15, 17.5, 20, 22.5, or 25 mg, wherein each combination is considered and intended to be individually described, and Compound (III) is administered in an amount of 800 mg a day.

When Compounds (I-III) are co-administered or closely sequentially administered their bioavailability is significantly enhanced. This unexpected result is highly advantageous in that increased distribution reduces the potential for treatment failure. Furthermore, lower concentrations of compound may be used which minimizes the potential for drug toxicity.

A drug-drug interaction study was carried out in human subjects to determine the interaction between Compounds (I-III) in vivo (see Example 1 below). The study concluded that SMV and ODV both increase exposure to Compound (III), however, Compound (III) does not alter the bioavailability of SMV or ODV. When SMV and ODV are co-administered with Compound (III), the bioavailability of Compound (III) was approximately 8-fold higher when compared to subjects given Compound (III) alone. Metabolites A-1 to A-5 were also present at higher concentrations when Compound (III) was administered with SMV and ODV. In addition, SMV was found to increase the exposure of ODV by 1.6-fold. Similarly, ODV also increased the bioavailability of SMV by 1.6-fold. Further studies concluded that ODV does not inhibit cytochrome P450 enzymes.

Thus, the combination of Compounds (I-III) is unexpectedly advantageous over single administration of any one of these three drugs. In fact, it could have been predicted that Simeprevir might adversely affect the pharmacokinetics of Compound (III), but that was not what was provided. Given that ODV does not inhibit CYP450 activity, it is not possible to predict that ODV and SMV would demonstrate superior pharmacokinetic properties when taken together.

In one embodiment, the present invention also provides a method of treating a hepatitis C infection in a patient comprising administering to the patient an effective amount of an approximately simultaneous, for example, fixed dosage, combination, comprising the above three active anti-HCV agents or independently their solvate, hydrate or pharmaceutically acceptable salt.

In certain embodiments, the targeted patient has cirrhosis of the liver. In other embodiments, the patient does not have cirrhosis of the liver. In other embodiments, the patient has hepatocellular carcinoma. In different embodiments, the HCV-infected patient does not have hepatocellular carcinoma.

The targeted patient may be infected with HCV genotype 1a, 1b, 1c, 2a, 2b, 2c, 3a, 3b, 4a, 5a or 6a, or a combination thereof. In another embodiment, the targeted patient may be infected with HCV genotype 1, 2, 3, 4, 5, or 6. In another embodiment, the targeted patient may be infected with HCV genotype 1, 4, or 2. In one embodiment, the patient is infected with genotype 1. In one embodiment, the patient is infected with genotype 2. In another embodiment, the patient is infected with genotype 3. In another embodiment, the patient is infected with genotype 4. In another embodiment, the patient is infected with genotype 4. In another embodiment, the patient is infected with genotype 5. In another embodiment, the patient is infected with genotype 6. In one embodiment, the treatment provides pan-genomic efficacy.

An open-label human study was conducted using a combination of Compound (III) and Odalasvir with or without Simeprevir for 6-8 weeks in 80 treatment-naïve, HCV genotype 1 infected patients without cirrhosis. These patients were divided into four groups (shown below). The following results provide a non-limiting example of the present invention:
(i) Compound (III) (400 mg), Odalasvir (50 mg) and Simeprevir (100 mg), given once a day for 8 weeks resulted in 20/20 (100%) patients with an SVR of 24 weeks.
(ii) Compound (III) (800 mg once a day), Odalasvir (50 mg every other day) without Simeprevir for 8 weeks resulted in 18/20 (90%) patients with an SVR of 12 weeks.
(iii) Compound (III) (800 mg once a day), Odalasvir (50 mg every other day) and Simeprevir (75 mg once a day) for 8 weeks resulted in 20/20 (100%) patients with an SVR of 12 weeks.
(iv) Compound (III) (800 mg once a day), Odalasvir (50 mg every other day) and Simeprevir (75 mg once a day) for 6 weeks resulted in 20/20 patients with an SVR of 12 weeks.

Consistent with the drug-drug interaction studies, increasing the Compound (III) dose from 400 to 800 mg increased the 5′-OH nucleoside metabolite A-1 exposure less than proportionally. Observed Odalasvir and Simeprevir exposures in group (i) were higher than anticipated. Reducing Odalasvir dosing from once a day to every other day decreased Odalasvir exposure proportionally. Reducing Simeprevir dosing from 100 mg to 75 mg QD decreased Simeprevir exposure less than proportionally.

In an alternative embodiment, the described combination of drugs may be administered as a prophylaxis to prevent HCV infection.

The invention also includes the specific combinations and dosage forms wherein Simeprevir may be in the form of an amorphous sodium salt, Odalasvir may be crystalline or amorphous that in some embodiments is not a salt, and Compound (III) may be an anhydrous crystalline form that in some embodiments is not a salt, hydrate or solvate. In an alternative embodiments, Odalasvir is provided as a hydrate, and in particular, a dihydrate.

### BRIEF DESCRIPTION OF THE FIGURES

As used in the figures Odalasvir is sometimes referred to as “ODV” and Simeprevir is sometimes referred to as “SMV”.

FIG. 1A is a graph of the Compound (III) concentration resulting from various dosage combinations as a function of time for Group 1. The y-axis is the Compound (III) concentration measured in nanograms/milliliter in blood and the x-axis is time in hours after human administration in vivo (see Example 1 below). The lower curve with diamond data points is the concentration curve obtained on day 3 with the dosing of Compound (III) alone. The middle curve with rectangle data points is the concentration curve obtained when Compound (III) and SMV were dosed on day 13. The upper curve with triangle data points is the concentration curve for Compound (III) when Compound (III), SMV and ODV were dosed on day 23.

FIG. 1B is a graph of the Compound (III) concentration resulting from various dosage combinations as a function of time for Group 2. The y-axis is the Compound (III) concentration measured in nanograms/milliliter in blood and the x-axis is time in hours after human administration in vivo. The lower curve with the diamond data points is the concentration curve obtained on day 3 with the dosing of Compound (III) alone. The middle curve with rectangle data points is the concentration curve obtained when Compound (III) and ODV were dosed on day 13. The upper curve with triangle data points is the concentration curve for Compound (III) when Compound (III), SMV and ODV were dosed on day 23.

FIG. 2A is a graph of the Compound A-3 concentration resulting from various dosage combinations as a function of time for Group 1. The y-axis is the Compound A-3 concentration measured in nanograms/milliliter in blood and the x-axis is time in hours after human administration in vivo. The lower curve with diamond data points is the concentration curve obtained on day 3 with the dosing of Compound (III) alone. The middle curve with rectangle data points is the concentration curve obtained when Compound (III) and SMV were dosed on day 13. The upper curve with triangle data points is the concentration curve obtained when Compound (III), SMV and ODV were dosed on day 23.

FIG. 2B is a graph of the Compound A-3 concentration resulting from various dosage combinations as a function of time for Group 2. The y-axis is the Compound A-3 concentration measured in nanograms/milliliter in blood and the x-axis is time in hours after human administration in vivo. The lower curve diamond data points is the concentration curve obtained on day 3 with the dosing of Compound (III) alone. The middle curve with rectangle data points is the concentration curve obtained when Compound (III) and ODV were dosed on day 13. The upper curve with triangle data points is the concentration curve obtained when Compound (III), SMV and ODV were dosed on day 23.

FIG. 3A is a graph of the Compound A-1 concentration resulting from various dosage combinations as a function of time for Group 1. The y-axis is the Compound A-1 concentration measured in nanograms/milliliter in blood and the x-axis is time in hours after human administration in vivo. The curve with diamond data points is the concentration curve obtained on day 3 with the dosing of Compound (III) alone. The curve with rectangle data points is the concentration curve obtained when Compound (III) and SMV were dosed on day 13. The curve with triangle data points is the concentration curve obtained when Compound (III), SMV and ODV were dosed on day 23.

FIG. 3B is a graph of the Compound A-1 concentration resulting from various dosage combinations as a function of time for Group 2. The y-axis is the Compound A-1 concentration measured in nanograms/milliliter in blood and the x-axis is time in hours after human administration in vivo. The lower curve diamond data points is the concentration curve obtained on day 3 with the dosing of Compound (III) alone. The middle curve with rectangle data points is the concentration curve obtained when Compound (III) and ODV were dosed on day 13. The upper curve with triangle data points is the concentration curve obtained when Compound (III), SMV and ODV were dosed on day 23.

FIG. 4A is a graph of the SMV concentration resulting from various dosage combinations as a function of time for Group 1. The y-axis is the SMV concentration measured in nanograms/milliliter in blood and the x-axis is time in hours after human administration in vivo. The curve with diamond data points is the concentration curve obtained on day 10 with the dosing of SMV alone. The curve with rectangle data points is the concentration curve obtained when Compound (III) and SMV were dosed on day 13. The curve with triangle data points is the concentration curve for SMV when SMV and ODV were dosed on day 20. The curve with the data points is the concentration curve for SMV when SMV, ODV and Compound (III) were dosed on day 23.

FIG. 4B is a graph of the SMV concentration resulting from various dosage combinations as a function of time for Group 2. The y-axis is the SMV concentration measured in nanograms/milliliter in blood and the x-axis is time in hours after human administration in vivo. The lower curve with triangle data points is the concentration curve obtained on day 20 with the dosing of SMV and ODV. The top curve with the [symbol] data points is the concentration curve obtained when SMV, ODV and Compound (III) were dosed on day 23.

FIG. 5A is a graph of the ODV concentration resulting from various dosage combinations as a function of time for Group 1. The y-axis is the ODV concentration measured in nanograms/milliliter in blood and the x-axis is time in hours after human administration in vivo. The curve with diamond data points is the concentration curve obtained on day 20 with the dosing of ODV and SMV. The curve with rectangle data points is the concentration curve obtained when Compound (III), SMV and ODV were dosed on day 23.

FIG. 5B is a graph of the ODV concentration resulting from various dosage combinations as a function of time for Group 1. The y-axis is the ODV concentration measured in nanograms/milliliter in blood and the x-axis is time in hours after human administration in vivo. The lower curve with diamond data points is the concentration curve obtained on day 10 with the dosing of ODV alone. The curve with rectangle data points is the concentration curve obtained when ODV and Compound (III) were dosed on day 13. The curve with triangle data points is the concentration curve for ODV when ODV and SMV were dosed on day 20. The curve with [symbol] data points is the concentration curve for ODV when Compound (III), SMV and ODV were dosed on day 23.

FIG. 6 is a graph of the Compound A-1 plasma concentration resulting from once daily administration of single oral doses of Compound (III), SMV, and ODV as a function of time. The y-axis is Compound A-1 plasma concentration measured in ng/mL and the x-axis is time measured in hours. The top curve is the plasma concentration curve obtained when Compound (III) is dosed at 800 mg. The bottom curve is the plasma concentration curve obtained when Compound (III) is dosed at 400 mg.

FIG. 7 is a graph of the Simeprevir (SMV) plasma concentration resulting from once daily administration of single oral doses of Compound (III), SMV, and ODV as a function of time. The y-axis is SMV plasma concentration measured in ng/mL and the x-axis is time measured in hours. The top curve is the plasma concentration curve obtained when Simeprevir is dosed at 100 mg. The bottom curve is the plasma concentration curve obtained when Simeprevir is dosed at 75 mg.

FIG. 8 is a graph of the Odalasvir (ODV) plasma concentration resulting from single oral doses of Compound (III), SMV, and ODV as a function of time. The y-axis is ODV plasma concentration measured in ng/mL and the x-axis is time measured in hours. The top curve is the plasma concentration curve obtained when Odalasvir is dosed daily at 50 mg. The bottom curve is the plasma concentration curve obtained when Odalasvir is dosed every other day at 50 mg.

FIG. 9 is a graph of the Compound (III) plasma concentration resulting from a single dose administered in vivo to healthy volunteers as single agents in the combination therapy (Arm 1) or a fixed dose combination (FDC) (Arm 2, Arm 3, and Arm 4). The y-axis is plasma concentration of Compound (III) measured in ng/mL and the x-axis is time measured in hours. The curve with the open circles is the concentration curve obtained when ODV, SMV, and Compound (III) are administered concurrently as separate single agents (Arm 1). The curve with the closed circles is the concentration curve when ODV, SMV, and Compound (III) are administered as a FDC containing SMV 100 mg+ODV 50 mg as a spray dried solid dispersion with HPMC-AS L in a ratio ODV/HPMC-AS L 1/1 w/w+Compound (III) 800 mg (Arm 2). The curve with the open squares is the concentration curve when ODV, SMV, and Compound (III) are administered as a FDC containing SMV 100 mg+ODV 50 mg as a spray dried solid dispersion with HPMC-AS L in a ratio ODV/HPMC-AS L 1/3 w/w+Compound (III) 800 mg (Arm 3). The curve with the closed squares is the concentration curve when ODV, SMV, and Compound (III) are administered as a FDC: SMV 100 mg+ODV 50 mg as a spray dried solid dispersion with copovidone and poloxamers+Compound (III) 800 mg (Arm 4).

FIG. 10 is a graph of the Simeprevir plasma concentration resulting from a single dose administered in vivo to healthy volunteers as single agents in the combination therapy (Arm 1) or a fixed dose combination (FDC) (Arm 2, Arm 3, and Arm 4). The y-axis is plasma concentration of SMV measured in ng/mL and the x-axis is time measured in hours. The curve with the open circles is the concentration curve obtained when ODV, SMV, and Compound (III) are administered concurrently as separate single agents (Arm 1). The curve with the closed circles is the concentration curve when ODV, SMV, and Compound (III) are administered as a FDC: SMV 100 mg+ODV 50 mg as a spray dried solid dispersion with HPMC-AS L in a ratio ODV/HPMC-AS L 1/1 w/w+Compound (III) 800 mg (Arm 2). The curve with the open squares is the concentration curve when ODV, SMV, and Compound (III) are administered as a FDC: SMV 100 mg+ODV 50 mg as a spray dried solid dispersion with HPMC-AS L in a ratio ODV/HPMC-AS L 1/3 w/w+Compound (III) 800 mg (Arm 3). The curve with the closed squares is the concentration curve when ODV, SMV, and Compound (III) are administered as a FDC: SMV 100 mg+ODV 50 mg as a spray dried solid dispersion with copovidone and poloxamers+Compound (III) 800 mg (Arm 4).

FIG. 11 is a graph of the Odalasvir plasma concentration resulting from a single dose administered in vivo to healthy volunteers as single agents in the combination therapy (Arm 1) or a fixed dose combination (FDC) (Arm 2, Arm 3, and Arm 4). The y-axis is plasma concentration of ODV measured in ng/mL and the x-axis is time measured in hours. The curve with the open circles is the concentration curve obtained when ODV, SMV, and Compound (III) are administered concurrently as separate single agents (Arm 1). The curve with the closed circles is the concentration curve when ODV, SMV, and Compound (III) are administered as a FDC: SMV 100 mg+ODV 50 mg as a spray dried solid dispersion with HPMC-AS L in a ratio ODV/HPMC-AS L 1/1 w/w+Compound (III) 800 mg (Arm 2). The curve with the open squares is the concentration curve when ODV, SMV, and Compound (III) are administered as a FDC: SMV 100 mg+ODV 50 mg as a spray dried solid dispersion with HPMC-AS L in a ratio ODV/HPMC-AS L 1/3 w/w+Compound (III) 800 mg (Arm 3). The curve with the closed squares is the concentration curve when ODV, SMV, and Compound (III) are administered as a FDC: SMV 100 mg+ODV 50 mg as a spray dried solid dispersion with copovidone and poloxamers+Compound (III) 800 mg (Arm 4).

FIG. 12 is a graph of the Compound A-1 plasma concentration resulting from a single dose administered in vivo to healthy volunteers as single agents in the combination therapy (Arm 1) or a fixed dose combination (FDC) (Arm 2, Arm 3, and Arm 4). The y-axis is plasma concentration of Compound A-1 measured in ng/mL and the x-axis is time measured in hours. The curve with the open circles is the concentration curve obtained when ODV, SMV, and Compound (III) are administered concurrently as separate single agents (Arm 1). The curve with the closed circles is the concentration curve when ODV, SMV, and Compound (III) are administered as a FDC: SMV 100 mg+ODV 50 mg as a spray dried solid dispersion with HPMC-AS L in a ratio ODV/HPMC-AS L 1/1 w/w+Compound (III) 800 mg (Arm 2). The curve with the open squares is the concentration curve when ODV, SMV, and Compound (III) are administered as a FDC: SMV 100 mg+ODV 50 mg as a spray dried solid dispersion with HPMC-AS L in a ratio ODV/HPMC-AS L 1/3 w/w+Compound (III) 800 mg (Arm 3). The curve with the closed squares is the concentration curve when ODV, SMV, and Compound (III) are administered as a FDC: SMV 100 mg+ODV 50 mg as a spray dried solid dispersion with copovidone and poloxamers+Compound (III) 800 mg (Arm 4).

FIG. 13 is a graph of Compound A-3 plasma concentration resulting from a single dose administered in vivo to healthy volunteers as single agents in the combination therapy (Arm 1) or a fixed dose combination (FDC) (Arm 2, Arm 3, and Arm 4). The y-axis is plasma concentration of Compound A-3 measured in ng/mL and the x-axis is time measured in hours. The curve with the open circles is the concentration curve obtained when ODV, SMV, and Compound (III) are administered concurrently as separate single agents (Arm 1). The curve with the closed circles is the concentration curve when ODV, SMV, and Compound (III) are administered as a FDC: SMV 100 mg+ODV 50 mg as a spray dried solid dispersion with HPMC-AS L in a ratio ODV/HPMC-AS L 1/1 w/w+Compound (III) 800 mg (Arm 2). The curve with the open squares is the concentration curve when ODV, SMV, and Compound (III) are administered as a FDC: SMV 100 mg+ODV 50 mg as a spray dried solid dispersion with HPMC-AS L in a ratio ODV/HPMC-AS L 1/3 w/w+Compound (III) 800 mg (Arm 3). The curve with the closed squares is the concentration curve when ODV, SMV, and Compound (III) are administered as a FDC: SMV 100 mg+ODV 50 mg as a spray dried solid dispersion with copovidone and poloxamers+Compound (III) 800 mg (Arm 4).

### DETAILED DESCRIPTION OF THE INVENTION

### Definitions

As used in the specification and in the claims, the term “comprising” may include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically carriers, and excludes other compounds.

All ranges disclosed herein are inclusive of the recited endpoint and independently combinable (for example, the range of “from 2 mg to 10 mg” is inclusive of the endpoints, 2 mg and 10 mg, and independently all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” or “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.”

Unless otherwise indicated, the term “about” refers to plus or minus 10% or the “about” may refer to plus or minus 10% of the indicated number. For example, “about 10%” may indicate a range of 9% to 11% and “about 1” may mean from 0.9 to 1.1. Other meanings of “about” may be apparent from the context, such as rounding off, so, for example “about 1” may also mean from 0.5 to 1.4. The term about is used without regard to the Doctrine of Equivalents, and is not intended to be a substitute for it.

In one embodiment, the term approximately is used interchangeably with “about”.

As used herein, “effective amount” refers to the amount of Compounds (I), (II), and (III), or any pharmaceutically acceptable salts thereof, that elicits the biological or medicinal response in a tissue system (e.g., blood, plasma, biopsy) or warm-blooded animal (e.g., human), that is being sought by a health care provider, which includes alleviation of the symptoms of the disease being treated.

As used herein, “treatment experienced” refers to a patient who has had at least one previous course of a non-direct-acting antiviral agent (“DAA”), interferon-based HCV therapy, with or without ribavirin.

As used herein, “treatment naïve” refers to the patient not having previously received treatment with any drug—investigational or approved—for HCV infection.

The term “viral relapsers” as used herein is a term known to those skilled in the art and stands for the number of patients, given as an absolute number or as a percentage of the treated patients, who did not achieve SVR12 at the end of the treatment period and have an HCV RNA level of greater than LLOQ during week 24 after the end of the treatment period.

### Active Compounds

The present invention provides a specific combination of drugs using a specific dosage regime that can achieve a sustained virological response in a human against a hepatitis C infection after approximately 8, 7, 6, 5, or even 4 or less weeks of treatment. In some embodiments, the treatment regime leads to a sustained virological response of approximately at least 12 weeks, at least 18 weeks or at least 24 weeks. In some embodiments, the treatment regime leads to a sustained virological response of 12 weeks, 18 weeks or 24 weeks. In one aspect, the treatment is accomplished with one pill or other dosage form a day for the treatment period.

The anti-HCV drugs used in this advantageous combination are:
The NS3/4A protease inhibitor Simeprevir (Compound I, SMV), or a pharmaceutically acceptable salt, hydrate or solvate thereof;
The NS5A inhibitor Odalasvir (Compound (II), ACH-3102, ODV), or a pharmaceutically acceptable salt, hydrate (including a dihydrate), or solvate thereof; and
The NS5B polymerase inhibitor Compound (III), which is isopropyl ((S)-(((2S,3S,4R,5R)-5-(2,4-dioxo-3,4-dihydropyrimidin-1-(2H-yl)-2-fluoro-3,4-dihydroxy-4-methyltetrahydrofuran-2yl)methoxy)(phenoxy)phosphoryl)alaninate, or a pharmaceutically acceptable salt, hydrate or solvate thereof.

Simeprevir (SMV) can be prepared according to methods known in the art, for example, those methods described in WO 2007/014926 (see e.g. Example 5). In some embodiments, Simeprevir is provided as its sodium salt. Simeprevir and its uses are also covered by U.S. Pat. Nos. 7,671,032; 8,148,399; 8,349,869; 8,741,926; 8,754,106; 9,040,562; and 9,353,103. Simeprevir was approved by the U.S. FDA in November 2013 and is marketed as Olysio in 150 mg oral capsules for the treatment of hepatitis C. See also WO 2010/097229 which describes a spray drying process to obtain the amorphous sodium salt.

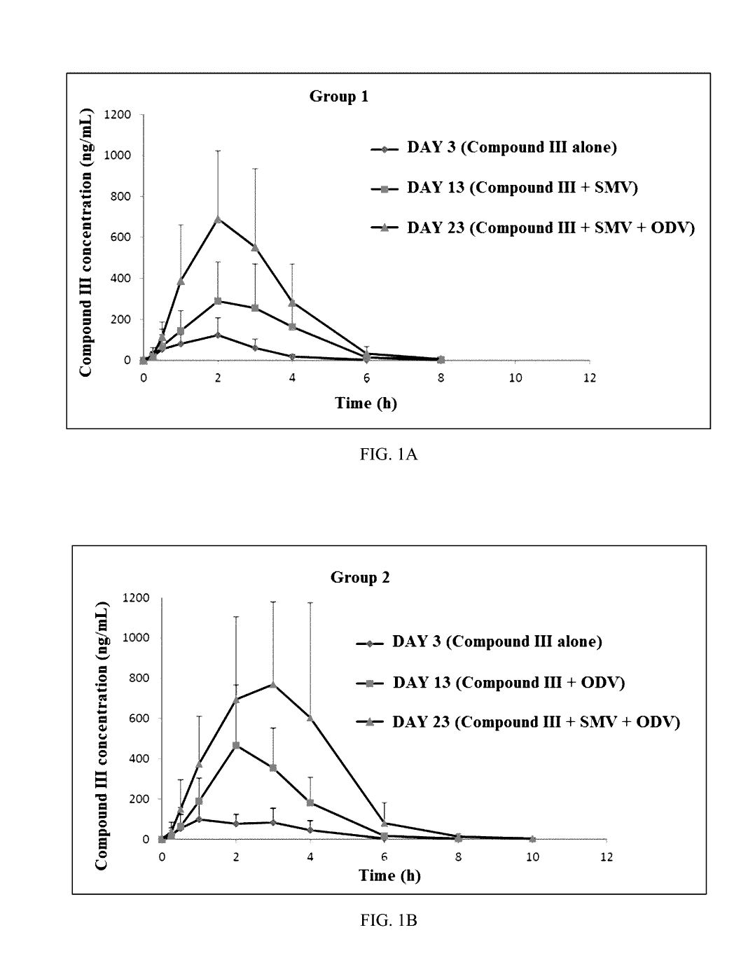

Odalasvir (ODV, ACH-3102) can be prepared according to methods known in the art, for example, those methods described in international patent application WO 2012/166716 (see e.g. compound number 43). In some embodiments, the form of ACH-3102 is a non-salt form, and in the same or other embodiments, it is an amorphous or crystalline form. The compound is described in U.S. Pat. No. 8,809,313. Odalasvir is also referred to as “ODV”, ACH-3102 and in FIGS. 1-5, it is referred to as “ODV”.

Compound (III) is an HCV RNA polymerase NS5B inhibitor. It can be prepared according to methods known in the art, for example, those methods described in WO 2014/100505 (see Example 31, compound 18). In some embodiments, the form of Compound (III) is a non-salt form; in the same or other embodiments, the form of compound (III) is not a solvate, whereas in still further embodiments it is a non-solvated crystalline form or an anhydrous crystalline form. Compound (III) is also described in U.S. Pat. Nos. 9,249,174 and 9,243,022 and Publication No.: U.S. 2015/0368286 (WO 2015/200216).

Compound (III) has the chemical structure:

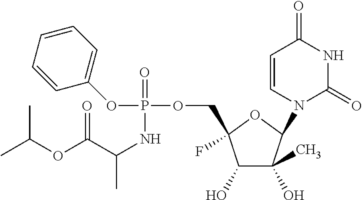 [SMILES: CC(C)OC(=O)C(C)NP(=O)(OC[C@@]1(F)O[C@@H](n2ccc(=O)[nH]c2=O)[C@](C)(O)[C@@H]1O)Oc1ccccc1]

In one embodiment, Compound (III) has the chemical structure:

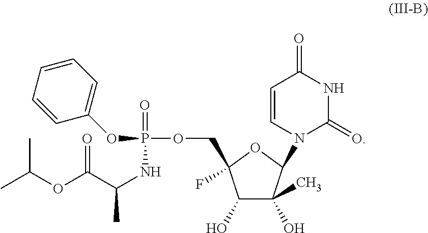 [SMILES: CC(C)OC(=O)[C@H](C)N[P@](=O)(OC[C@@]1(F)O[C@@H](n2ccc(=O)[nH]c2=O)[C@](C)(O)[C@@H]1O)Oc1ccccc1]

In one embodiment Compound (III) is isopropyl ((S)-(((2S,3S,4R,5R)-5-(2,4-dioxo-3,4-dihydropyrimidin-1 (2H)-yl)-2-fluoro-3,4-dihydroxy-4-methyltetrahydrofuran-2-yl)methoxy)(phenoxy)phosphoryl)-L-alaninate, or a pharmaceutically acceptable salt thereof. Compound (III) is typically provided in the form of Compound (III-B). Compound (III) is used in the examples to refer to Compound (III-B).

In one embodiment, Compound (III) has the chemical structure:

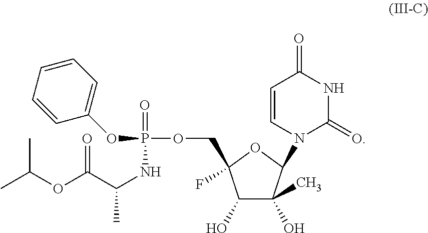 [SMILES: CC(C)OC(=O)[C@@H](C)N[P@](=O)(OC[C@@]1(F)O[C@@H](n2ccc(=O)[nH]c2=O)[C@](C)(O)[C@@H]1O)Oc1ccccc1]

In one embodiment Compound (III) is isopropyl ((S)-(((2S,3S,4R,5R)-5-(2,4-dioxo-3,4-dihydropyrimidin-1(2H)-yl)-2-fluoro-3,4-dihydroxy-4-methyltetrahydrofuran-2-yl)methoxy)(phenoxy)phosphoryl)-D-alaninate, or a pharmaceutically acceptable salt thereof.

In one embodiment, Compound (III) has the chemical structure:

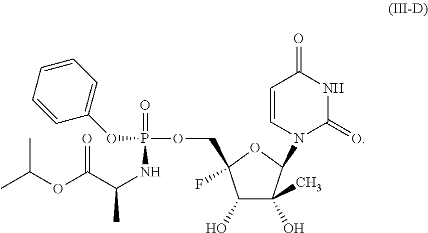 [SMILES: CC(C)OC(=O)[C@H](C)N[P@@](=O)(OC[C@@]1(F)O[C@@H](n2ccc(=O)[nH]c2=O)[C@](C)(O)[C@@H]1O)Oc1ccccc1]

In one embodiment Compound (III) is isopropyl ((R)-(((2S,3S,4R,5R)-5-(2,4-dioxo-3,4-dihydropyrimidin-1(2H)-yl)-2-fluoro-3,4-dihydroxy-4-methyltetrahydrofuran-2-yl)methoxy)(phenoxy)phosphoryl)-L-alaninate, or a pharmaceutically acceptable salt thereof.

In one embodiment Compound (III) has the chemical structure:

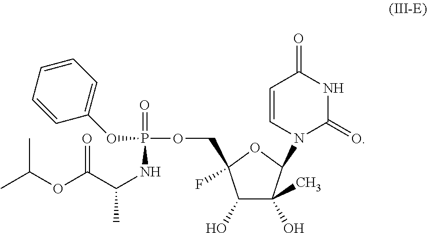 [SMILES: CC(C)OC(=O)[C@@H](C)N[P@@](=O)(OC[C@@]1(F)O[C@@H](n2ccc(=O)[nH]c2=O)[C@](C)(O)[C@@H]1O)Oc1ccccc1]

In one embodiment Compound (III) is isopropyl ((R)-(((2S,3S,4R,5R)-5-(2,4-dioxo-3,4-dihydropyrimidin-1 (2H)-yl)-2-fluoro-3,4-dihydroxy-4-methyltetrahydrofuran-2-yl)methoxy)(phenoxy)phosphoryl)-D-alaninate, or a pharmaceutically acceptable salt thereof.

In one embodiment, the phosphoramidate has an (S)-chiral phosphorus and the amino acid of the phosphoramidate is in the L-configuration. In another embodiment, the phosphoramidate has an (S)-chiral phosphorus and the amino acid of the phosphoramidate is in the D-configuration.

In one embodiment the phosphoramidate is isopropyl ((S)-ethoxy(phenoxy)phosphoryl)-L-alaninate.

Compound (III) is provided in the form of a phenoxy, isopropyl-alaninate phosphoramidate ester prodrug of the 2′-methyl, 2′-hydroxyl, 3′-hydroxy, 4′-fluorouridine nucleoside. The phosphoramidate prodrug facilitates the metabolism of the nucleoside to the active 5′-triphosphate, by maximizing the amount of intracellular 5′-monophosphate metabolite which is readily anabolized in vivo to the 5′-triphosphate. A-4 is not a circulating metabolite and thus not directly measured as it would require a biopsy of the liver.

Compound (III) is metabolized through A-3 to A-2 primarily via A-4 and A-5 with some production of A-1 (see the structures below).

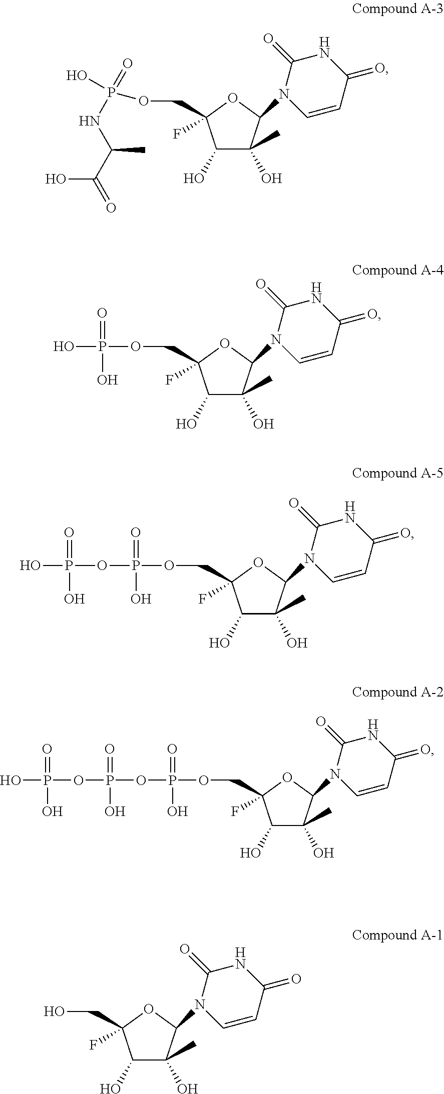 [SMILES: C[C@H](NP(=O)(O)OC[C@@]1(F)O[C@@H](n2ccc(=O)[nH]c2=O)[C@](C)(O)[C@@H]1O)C(=O)O ; C[C@]1(O)[C@H](n2ccc(=O)[nH]c2=O)O[C@](F)(COP(=O)(O)O)[C@H]1O ; C[C@]1(O)[C@H](n2ccc(=O)[nH]c2=O)O[C@](F)(COP(=O)(O)OP(=O)(O)O)[C@H]1O ; C[C@]1(O)[C@H](n2ccc(=O)[nH]c2=O)O[C@](F)(COP(=O)(O)OP(=O)(O)OP(=O)(O)O)[C@H]1O ; C[C@]1(O)[C@H](n2ccc(=O)[nH]c2=O)O[C@](F)(CO)[C@H]1O]

### Dosage Forms of the Anti-HCV Combination Treatment

The invention includes pharmaceutical dosage forms that provide the described active compounds in an effective amount in combination to treat a patient, typically a human, infected with HCV. In certain embodiments, Simeprevir is administered once a day in a dosage of approximately 75 or 100 mg optionally as its sodium salt (wherein the mg weight refers to the weight of active compound without regard to the weight of the salt). In alternative embodiments, Simeprevir can optionally be used, for example, in amorphous or crystalline form and/or as a hydrate, solvate and/or in a pharmaceutically acceptable carrier.

Odalasvir is administered once a day in a dosage of at least approximately 5, 7.5, 10, 12.5, 15, 17.5, 20, 22.5, 25 or 50 mg. Odalasvir can be used, for example, in amorphous or crystalline form and/or as a hydrate (including a dihydrate), a solvate or a pharmaceutically acceptable salt. In one embodiment, Odalasvir is provided in the dosage form, or combination of dosage forms, in an amount of 10, 12.5 or 15 mg per day. In one embodiment, Odalasvir is dosed in an amount of 20 or 25 mg every day.

Compound (III) is administered in an amount of 400, 600, 700, 800, 900, or 1000 mg a day. Compound (III) can be provided optionally in amorphous or crystalline form or as a hydrate, a solvate or a pharmaceutically acceptable salt.

In one specific embodiment, Simeprevir is administered once a day in a dosage of approximately 75 or 100 mg as its sodium salt, Odalasvir is administered once a day in a dosage of approximately 10, 12.5, 17.5 or 20 or 25 mg, and Compound (III) is administered in an amount of 800 mg a day.

In one aspect, these three active compounds are administered in a single fixed dosage form once a day, which may have the benefit of improving treatment compliance and has the unexpected benefit of advantageous drug-drug interaction for phaarmokinetic. In another embodiment, the three drugs are formulated together into two or more fixed dosage forms, which are taken simultaneously or over the course of the day, for example two or three times a day, as prescribed by the healthcare provider. In yet another embodiment, the three active anti-HCV drugs are provided in separate pills and are administered approximately simultaneously. In another aspect, two of the three drugs are provided in a fixed dose combination and the third is provided in a separate dosage form but administered approximately simultaneously. In one embodiment, one or more of the active compounds is administered every other day, for example, Odalasvir.

The invention also includes the specific combinations that include dosage forms wherein Simeprevir may be in the form of an amorphous sodium salt, Odalasvir may be a crystalline or amorphous form that in some embodiments is not in the form of a salt, and Compound (III) may be an anhydrous crystalline form that in some embodiments is not in the form of a salt or solvate.

It has been unexpectedly discovered that co-administration of effective amounts of Compounds I, II and III any of which can be in the form of a pharmaceutically acceptable salt, leads to an unexpectedly advantageous effect on the pharmacokinetics/dynamics of the drug combination which can allow for a reduction in the treatment time and/or effective treatment dosages over what would be predicted according to conventional treatment methods and compounds. For example, it has surprisingly and unexpectedly been discovered that the combination of these three drugs improves the bioavailability of the drug combination on administration.

A human in vivo study (see Example 1) has confirmed that: (i) exposure to Compound (III) (the phosphoramidate prodrug of the nucleoside) increases by 7 to 8 times; (ii) exposure to Compound A-3 (the de-esterified phosphoramidate prodrug of the nucleoside, which is the first step of metabolism) increases by 1.9 to 2.8 times; (iii) exposure to Compound A-1 (the parent nucleoside with a free 5′-OH group) increases by 1 to 1.5 times; (iv) exposure to Simeprevir and Odalasvir interaction appears additive and increases the Cₘᵢₙ of Compound A-1 by 3 to 3.5 times; (v) exposure of Simeprevir increases by 1.6; and (vi) exposure of Odalasvir increases by 1.5 times.

Thus, the combination of these three drugs is unexpectedly advantageous over the single administration of any one of the drugs.

In some embodiments, the Compounds (I), (II), and (III), or independently a pharmaceutically acceptable salt, hydrate or solvate thereof are administered as separate oral capsules or oral tablets. Formulations may include solid dispersions, including a spray dried dispersion.

When a combination is referred to herein, specifically a combination of the compounds of Compounds (I), (II) and (III), or independently any pharmaceutically acceptable hydrate, solvate or salt of a component thereof, such a combination may be a single formulation comprising all three compounds or it may be a combination product (such a kit of parts) where each of the three compounds may be packaged together either as three separate forms (each comprising an active substance) or as two forms (one form comprising any two of the active substances, and the other form comprising the remaining active substance), wherein active substance refers to any of Compounds (I), (II) and (III) or independently a pharmaceutically acceptable hydrate, solvate or salt thereof. The combination of compounds as described herein may be co-administered, sequentially administered, or administered substantially simultaneously. Hence the individual dosage forms of each of compounds (I), (II), and (III), or independently a pharmaceutically acceptable hydrate, solvate or salt thereof can be administered as separate forms (e.g., as separate tablets or capsules) as described herein or, in other embodiments, may be administered as a single form containing all three active substances or as two forms (one containing any two of the active substances and the other containing the remaining active substance).

All amounts mentioned in this disclosure refer to the free form (i.e. non-salt, hydrate or solvate form). The values given below represent free-form equivalents, i.e., quantities as if the free form would be administered. If salts are administered the amounts need to be calculated in function of the molecular weight ratio between the salt and the free form.

The daily doses described herein are calculated for an average body weight of about 70 kg and may be recalculated in case of pediatric applications, or when used with patients with a substantially diverting body weight, according to the advice of the healthcare practitioner.

In some embodiments, Compound (I) (Simeprevir), or a pharmaceutically acceptable salt thereof, is administered in an amount that is about 50 mg to about 200 mg per day. For example, Compound (I) (Simeprevir), or a pharmaceutically acceptable salt thereof, is administered in an amount that is at least about 50, 60, 70, 75, 80, 90, 100, 110, 120, 130, 140, 150, 160, 170, 180, 190, or 200 mg per day (for example 150 mg, 100 mg or 75 mg per day). In certain embodiments, Compound (I), or a pharmaceutically acceptable salt thereof, is administered in an amount that is about 150 mg per day (for the duration of the treatment regimen). In still other embodiments Compound (I), or pharmaceutically acceptable salt thereof, is administered in an amount that is about 100 mg per day (such a dose is lower than the daily 150 mg dose, for use in combination, approved in e.g. US and the EU). In another embodiment, Compound (I), or a pharmaceutically acceptable salt thereof, is administered in an amount that is about 75 mg per day. In certain of these embodiments, Simeprevir is administered as the sodium salt.

In some embodiments, Compound (II) (Odalasvir), or a pharmaceutically acceptable salt thereof, is administered in an amount that is about 10 mg to about 200 mg per day. For example, Compound (II), or a pharmaceutically acceptable salt thereof, is administered in an amount that is at least about 5, 10, 12.5, 15, 17.5, 20, 25, 50, 75, 100, 125, 150, 175, or 200 mg per day (e.g. 10 mg or 12.5 mg or 25 mg or 50 mg per day). In some embodiments, Compound (II), or a pharmaceutically acceptable salt thereof, is administered in an amount that is about 12.5 mg per day. In some embodiments, Compound (II), or a pharmaceutically acceptable salt thereof, is administered in an amount that is about 25 mg per day. Still other embodiments include those in which Compound (II) is administered: (i) once daily in an amount that is about 50 mg; or (ii) in an amount of about 150 mg as a loading dose and thereafter once daily in about 50 mg, each for the duration of the treatment regimen. In yet other embodiments, Odalasvir is administered every other day, for example, 50 mg every other day (“QOD”).

In some embodiments, Compound (III), or a pharmaceutically acceptable salt thereof, is administered in an amount that is about 200 mg to about 1200 mg per day. For example, Compound (III), or a pharmaceutically acceptable salt thereof, is administered in an amount that is about 200, 250, 300, 350, 400, 450, 500, 550, 600, 650, 700, 750, 800, 850, 900, 950, 1000, 1050, 1100, 1150 or 1200 mg per day (e.g. 400 mg, 600 mg, 800 mg or 1200 mg per day). In some embodiments, Compound (III), or a pharmaceutically acceptable salt thereof, is administered in an amount that is about 800 mg per day (for the duration of the treatment regimen). In still another embodiment, Compound (III), or pharmaceutically acceptable salt thereof, is administered in an amount that is about 400 mg per day.

The combination of Compounds as described herein may be co-administered, sequentially administered, or administered substantially simultaneously (as described herein). Hence the individual dosage forms of each of the Compounds (I), (II), and (III), or any pharmaceutical salts thereof can be administered as separate forms (e.g. as separate tablets or capsules) as described herein or, in an alternative embodiment, may be administered as a single form containing all three actives or as two forms (one containing any two of the actives and the other containing the remaining active).

In one embodiment, the combination of compounds as described herein are administered as a single tablet that contains 75 or 100 mg of Simeprevir, or a pharmaceutically acceptable salt thereof; 5 mg of Odalasvir or a pharmaceutically acceptable salt thereof; and 800 mg of Compound (III).

In one embodiment, the combination of compounds as described herein are administered as a single tablet that contains 75 or 100 mg of Simeprevir, or a pharmaceutically acceptable salt thereof; 7.5 mg of Odalasvir or a pharmaceutically acceptable salt thereof; and 800 mg of Compound (III).

In one embodiment, the combination of compounds as described herein are administered as a single tablet that contains 75 or 100 mg of Simeprevir, or a pharmaceutically acceptable salt thereof; 10 mg of Odalasvir or a pharmaceutically acceptable salt thereof; and 800 mg of Compound (III).

In one embodiment, the combination of compounds as described herein are administered as a single tablet that contains 75 or 100 mg of Simeprevir, or a pharmaceutically acceptable salt thereof; 12.5 mg of Odalasvir or a pharmaceutically acceptable salt thereof; and 800 mg of Compound (III).

In one embodiment, the combination of compounds as described herein are administered as a single tablet that contains 75 or 100 mg of Simeprevir, or a pharmaceutically acceptable salt thereof; 15 mg of Odalasvir or a pharmaceutically acceptable salt thereof; and 800 mg of Compound (III).

In one embodiment, the combination of compounds as described herein are administered as a single tablet that contains 75 or 100 mg of Simeprevir, or a pharmaceutically acceptable salt thereof; 20 mg of Odalasvir or a pharmaceutically acceptable salt thereof; and 800 mg of Compound (III).

In one embodiment, the combination of compounds as described herein are administered as a single tablet that contains 75 or 100 mg of Simeprevir, or a pharmaceutically acceptable salt thereof; 25 mg of Odalasvir or a pharmaceutically acceptable salt thereof; and 800 mg of Compound (III).

In one embodiment, the combination of compounds as described herein are administered as a single tablet that contains 75 or 100 mg of Simeprevir, or a pharmaceutically acceptable salt thereof; 50 mg of Odalasvir or a pharmaceutically acceptable salt thereof; and 800 mg of Compound (III).

In one embodiment, the combination of compounds as described herein is administered as a single tablet that contains 75 or 100 mg of Simeprevir, or a pharmaceutically acceptable salt thereof; 75 mg of Odalasvir or a pharmaceutically acceptable salt thereof; and 800 mg of Compound (III).

In one embodiment, the combination of compounds as described herein are administered as a single tablet that contains 75 or 100 mg of Simeprevir, or a pharmaceutically acceptable salt thereof; 100 mg of Odalasvir or a pharmaceutically acceptable salt thereof; and 800 mg of Compound (III).

In one embodiment, the combination of compounds as described herein are administered as a single tablet that contains 75 or 100 mg of Simeprevir, or a pharmaceutically acceptable salt thereof; 125 mg of Odalasvir or a pharmaceutically acceptable salt thereof; and 800 mg of Compound (III).

In one embodiment, the combination of compounds as described herein are administered as a single tablet that contains 75 or 100 mg of Simeprevir, or a pharmaceutically acceptable salt thereof; 150 mg of Odalasvir or a pharmaceutically acceptable salt thereof; and 800 mg of Compound (III).

In one embodiment, the combination of compounds as described herein are administered as a single tablet that contains 75 or 100 mg of Simeprevir, or a pharmaceutically acceptable salt thereof; 200 mg of Odalasvir or a pharmaceutically acceptable salt thereof; and 800 mg of Compound (III).

In one embodiment, the combination of compounds as described herein are co-administered as two tablets that contain 75 or 100 mg of Simeprevir, or a pharmaceutically acceptable salt thereof; 10 mg of Odalasvir or a pharmaceutically acceptable salt thereof; and 800 mg of Compound (III).

In one embodiment, the combination of compounds as described herein are co-administered as two tablets that contain 75 or 100 mg of Simeprevir, or a pharmaceutically acceptable salt thereof; 12.5 mg of Odalasvir or a pharmaceutically acceptable salt thereof; and 800 mg of Compound (III).

In one embodiment, the combination of compounds as described herein are co-administered as two tablets that contain 75 or 100 mg of Simeprevir, or a pharmaceutically acceptable salt thereof; 15 mg of Odalasvir or a pharmaceutically acceptable salt thereof; and 800 mg of Compound (III).

In one embodiment, the combination of compounds as described herein are co-administered as two tablets that contain 75 or 100 mg of Simeprevir, or a pharmaceutically acceptable salt thereof; 20 mg of Odalasvir or a pharmaceutically acceptable salt thereof; and 800 mg of Compound (III).

In one embodiment, the combination of compounds as described herein are co-administered as two tablets that contain 75 or 100 mg of Simeprevir, or a pharmaceutically acceptable salt thereof; 25 mg of Odalasvir or a pharmaceutically acceptable salt thereof; and 800 mg of Compound (III).

In one embodiment, the combination of compounds as described herein are co-administered as two tablets that contain 75 or 100 mg of Simeprevir, or a pharmaceutically acceptable salt thereof; 50 mg of Odalasvir or a pharmaceutically acceptable salt thereof; and 800 mg of Compound (III).

In one embodiment, the combination of compounds as described herein are co-administered as two tablets that contain 75 or 100 mg of Simeprevir, or a pharmaceutically acceptable salt thereof; 75 mg of Odalasvir or a pharmaceutically acceptable salt thereof; and 800 mg of Compound (III).

In one embodiment, the combination of compounds as described herein are co-administered as two tablets that contain 75 or 100 mg of Simeprevir, or a pharmaceutically acceptable salt thereof; 100 mg of Odalasvir or a pharmaceutically acceptable salt thereof; and 800 of Compound (III).

In one embodiment, the combination of compounds as described herein are co-administered as two tablets that contain 75 or 100 mg of Simeprevir, or a pharmaceutically acceptable salt thereof; 125 mg of Odalasvir or a pharmaceutically acceptable salt thereof; and 800 mg of Compound (III).

In one embodiment, the combination of compounds as described herein are co-administered as two tablets that contain 75 or 100 mg of Simeprevir, or a pharmaceutically acceptable salt thereof; 150 mg of Odalasvir or a pharmaceutically acceptable salt thereof; and 800 mg of Compound (III).

In one embodiment, the combination of compounds as described herein are co-administered as two tablets that contain 75 or 100 mg of Simeprevir, or a pharmaceutically acceptable salt thereof; 175 mg of Odalasvir or a pharmaceutically acceptable salt thereof; and 800 mg of Compound (III).

In one embodiment, the combination of compounds as described herein are co-administered as two tablets that contain 75 or 100 mg of Simeprevir, or a pharmaceutically acceptable salt thereof; 200 mg of Odalasvir or a pharmaceutically acceptable salt thereof; and 800 mg of Compound (III).

In one embodiment, the combination of compounds as described herein are sequentially administered as two tablets that contain 75 or 100 mg of Simeprevir, or a pharmaceutically acceptable salt thereof; 10 mg of Odalasvir or a pharmaceutically acceptable salt thereof; and 800 mg of Compound (III).

In one embodiment, the combination of compounds as described herein are sequentially administered as two tablets that contain 75 or 100 mg of Simeprevir, or a pharmaceutically acceptable salt thereof; 12.5 mg of Odalasvir or a pharmaceutically acceptable salt thereof; and 800 mg of Compound (III).

In one embodiment, the combination of compounds as described herein are sequentially administered as two tablets that contain 75 or 100 mg of Simeprevir, or a pharmaceutically acceptable salt thereof; 15 mg of Odalasvir or a pharmaceutically acceptable salt thereof; and 800 mg Compound (III).

In one embodiment, the combination of compounds as described herein are sequentially administered as two tablets that contain 75 or 100 mg of Simeprevir, or a pharmaceutically acceptable salt thereof; 20 mg of Odalasvir or a pharmaceutically acceptable salt thereof; and 800 mg of Compound (III).

In one embodiment, the combination of compounds as described herein are sequentially administered as two tablets that contain 75 or 100 mg of Simeprevir, or a pharmaceutically acceptable salt thereof; 25 mg of Odalasvir or a pharmaceutically acceptable salt thereof; and 800 mg of Compound (III).

In one embodiment, the combination of compounds as described herein are sequentially administered as two tablets that contain 75 or 100 mg of Simeprevir, or a pharmaceutically acceptable salt thereof; 50 mg of Odalasvir or a pharmaceutically acceptable salt thereof; and 800 mg of Compound (III).

In one embodiment, the combination of compounds as described herein are sequentially administered as two tablets that contain 75 or 100 mg of Simeprevir, or a pharmaceutically acceptable salt thereof; 75 mg of Odalasvir or a pharmaceutically acceptable salt thereof; and 800 mg of Compound (III).

In one embodiment, the combination of compounds as described herein are sequentially administered as two tablets that contain 75 or 100 mg of Simeprevir, or a pharmaceutically acceptable salt thereof; 100 mg of Odalasvir or a pharmaceutically acceptable salt thereof; and 800 mg of Compound (III).

In one embodiment, the combination of compounds as described herein are sequentially administered as two tablets that contain 75 or 100 mg of Simeprevir, or a pharmaceutically acceptable salt thereof; 125 mg of Odalasvir or a pharmaceutically acceptable salt thereof; and 800 mg of Compound (III).

In one embodiment, the combination of compounds as described herein are sequentially administered as two tablets that contain 75 or 100 mg of Simeprevir, or a pharmaceutically acceptable salt thereof; 150 mg of Odalasvir or a pharmaceutically acceptable salt thereof; and 800 mg of Compound (III).

In one embodiment, the combination of compounds as described herein are sequentially administered as two tablets that contain 75 or 100 mg of Simeprevir, or a pharmaceutically acceptable salt thereof; 175 mg of Odalasvir or a pharmaceutically acceptable salt thereof; and 800 mg of Compound (III).

In one embodiment, the combination of compounds as described herein are sequentially administered as two tablets that contain 75 or 100 mg of Simeprevir, or a pharmaceutically acceptable salt thereof; 200 mg of Odalasvir or a pharmaceutically acceptable salt thereof; and 800 mg of Compound (III).

In one embodiment, the treatment includes 100 mg of Simeprevir, 50 mg of Odalasvir and 800 mg of Compound (III), any of which may be in the form of a pharmaceutically acceptable salt.

In some embodiments, the following amounts of active therapeutic agent are employed daily in the treatment regime: Simeprevir (150 mg, 100 mg or 75 mg), Odalasvir (50 mg or 25 mg), Compound (III) (800 mg or 400 mg; administered e.g. as 8×100 mg or 4×100 mg tablets/capsules or as 2×400 mg or 1×400 mg tablet/capsule or 1×800 mg). It will be understood that such amounts refer only to the weights of the non-salt moieties; if such active substances are formulated in a certain salt form (e.g. Simeprevir sodium salt, etc), the net weight of that part will proportionately increase. Further, it will also be understood that the active substances are in some embodiments formulated into the relevant tablets, for example with (a) pharmaceutically acceptable carrier(s) and/or excipient(s).

The in vitro antiviral activity against HCV of described combinations can be tested in a cellular HCV replicon system based on Lohmann et al. (1999) Science 285:110-113, with the further modifications described by Krieger, et al., (2001) Journal of Virology 75: 4614-4624 (incorporated herein by reference). This model, while not a complete infection model for HCV, is accepted as a robust and efficient model of autonomous HCV RNA replication. The in vitro antiviral activity against HCV can also be tested by enzymatic tests.

Compounds (I), (II) and (III), as described herein, may be used in pharmaceutically acceptable salt forms or in free (i.e. non-salt) form (or as a hydrate or solvate). Salt forms can be obtained by treating the free form with an acid or base to yield what is sometimes referred to as pharmaceutically acceptable acid and base addition salts. Pharmaceutically acceptable acid or base, as appropriate, addition salts of the Compounds (I), (II) and/or (III) can conveniently be obtained by treating the free form with an appropriate acid or base. Acids that are known to be useful in the formation of pharmaceutically acceptable salts comprise, for example, inorganic acids such as hydrohalic acids, such as hydrobromic acid, or in particular hydrochloric acid; or sulfuric, nitric, phosphoric and the like acids; or organic acids such as, for example, acetic, propanoic, hydroxyacetic, lactic, pyruvic, oxalic, malonic, succinic, maleic, fumaric, malic (i.e. hydroxybutanedioic acid), tartaric, citric, methanesulfonic, ethanesulfonic, benzenesulfonic, p-toluenesulfonic, cyclamic, salicylic, p-amino-salicylic, pamoic and the like acids. Bases that are known to be useful in the formation of pharmaceutically acceptable salts include pharmaceutically acceptable inorganic and organic bases, such as metal bases and amines, and illustratively among them, bases that lead to the formation of ammonium salts, alkali and earth alkaline metal salts, e.g. the lithium, sodium or potassium salts; or the magnesium or calcium salts; benzathine salts, N-methyl-D-glucamine salts, hydrabamine salts, and salts with amino acids such as, for example, arginine, lysine, and the like. It will also be understood that some embodiments of this invention also comprise any solvates that the Compounds of (I), (II) or (III) may form. Such solvates may be, for example, hydrates, alcoholates, e.g. ethanolates, and the like.

In an aspect of the invention, pharmaceutical compositions according to the present invention include one, two or three of the active agents described herein in combination with a pharmaceutically acceptable carrier, additive, or excipient, further optionally in combination or alternation with at least one of the other active compounds.

In general, while it is preferable to administer the pharmaceutical composition in orally-administrable form (such as a tablet, pill or gel-cap), the Compounds or their salts alone or by combination may be administered via a parenteral, intravenous, intramuscular, topical, transdermal, buccal, subcutaneous, suppository, or other route, including intranasal spray. Intravenous and intramuscular formulations are often administered in sterile saline. One of ordinary skill in the art may modify the formulations to render them more soluble in water or other vehicle, for example, this can be easily accomplished by minor modifications (salt formulation, esterification, etc.) which are well within the ordinary skill in the art. Given the disclosure herein, one can modify the route of administration and dosage regimen of a particular compound in order to manage the pharmacokinetics of the present compounds for maximum anti-HCV beneficial effect in patients.

To prepare the pharmaceutical compositions according to the present invention, a therapeutically effective amount of one or more of the compounds according to the present invention is often intimately admixed with a pharmaceutically acceptable carrier according to conventional pharmaceutical compounding techniques to produce a fixed dosage form. A carrier may take a wide variety of forms depending on the form of preparation desired for administration, e.g., oral or parenteral. In preparing pharmaceutical compositions in oral dosage form, any of the usual pharmaceutical media may be used. For solid oral preparations such as powders, tablets, capsules, and for solid preparations such as suppositories, suitable carriers and additives including starches, sugar carriers, such as dextrose, manifold, lactose, and related carriers, diluents, granulating agents, lubricants, binders, disintegrating agents, and the like may be used. If desired, the tablets or capsules may be enteric-coated or sustained release by standard techniques. The use of these dosage forms may significantly enhance the bioavailability of the compounds in the patient. Thus, for liquid oral preparations such as suspensions, elixirs, and solutions, suitable carriers and additives including water, glycols, oils, alcohols, flavoring agents, preservatives, coloring agents, and the like may be used.

For parenteral formulations, the carrier will usually comprise sterile water or aqueous sodium chloride solution, though other ingredients, including those which aid dispersion, also may be included. Of course, where sterile water is to be used and maintained as sterile, the compositions and carriers must also be sterilized. Injectable suspensions may also be prepared, in which case appropriate liquid carriers, suspending agents, and the like may be employed.

Liposomal suspensions (including liposomes targeted to viral antigens) may be prepared by conventional methods to produce pharmaceutically acceptable carriers. This may be appropriate for the delivery of free nucleosides, acyl/alkyl nucleosides or phosphate ester pro-drug forms of the nucleoside compounds according to the present invention.

In typical embodiments according to the present invention, the compounds and compositions are used to treat, prevent or delay an HCV infection or a secondary disease state, condition or complication of an HCV infection.

### Methods of Treatment

The present disclosure provides an unexpectedly advantageous combination of Simeprevir (Compound (I)), or a pharmaceutically acceptable salt, hydrate, or solvate thereof; Odalasvir (Compound (II)), or a pharmaceutically acceptable salt, hydrate, or solvate thereof; and Compound (III), or a pharmaceutically acceptable salt thereof, for use to treat a patient, typically a human, infected with hepatitis C using a treatment regime that terminates after a period of time that is approximately, 8 weeks or less, 7 weeks or less, 6 weeks or less, 6 weeks or less, 5 weeks or less, or 4 weeks or less.

In some embodiments, the administration of the compounds of compounds (I), (II), and (III), or any salt, hydrate or solvate form(s) thereof, terminates after a period of time that is less than 6 weeks, for example, 5, or 4 weeks. In other embodiments, the administration terminates after a period of time that is 4 weeks.

In one embodiment, the administration terminates after a period of time that is approximately 8 weeks, 7 weeks, 6 weeks, 5 weeks or 4 weeks or less.

In one embodiment, the administration terminates after a period of time of about 4 weeks to achieve an SVR of at least 12 weeks.

In one embodiment, the administration terminates after a period of time of about 4 weeks to achieve an SVR of at least 18 weeks.

In one embodiment, the administration terminates after a period of time of about 4 weeks to achieve an SVR of at least 24 weeks.

In one embodiment, the administration terminates after a period of time of about 5 weeks to achieve an SVR of at least 12 weeks.

In one embodiment, the administration terminates after a period of time of about 5 weeks to achieve an SVR of at least 18 weeks.

In one embodiment, the administration terminates after a period of time of about 5 weeks to achieve an SVR of at least 24 weeks.

In one embodiment, the administration terminates after a period of time of about 6 weeks to achieve an SVR of at least 12 weeks.

In one embodiment, the administration terminates after a period of time of about 6 weeks to achieve an SVR of at least 18 weeks.

In one embodiment, the administration terminates after a period of time of about 6 weeks to achieve an SVR of at least 24 weeks.

In one embodiment, the administration terminates after a period of time of about 7 weeks to achieve an SVR of at least 12 weeks.

In one embodiment, the administration terminates after a period of time of about 7 weeks to achieve an SVR of at least 18 weeks.

In one embodiment, the administration terminates after a period of time of about 7 weeks to achieve an SVR of at least 24 weeks.

In one embodiment, the administration terminates after a period of time of about 8 weeks to achieve an SVR of at least 12 weeks.

In one embodiment, the administration terminates after a period of time of about 8 weeks to achieve an SVR of at least 18 weeks.

In one embodiment, the administration terminates after a period of time of about 8 weeks to achieve an SVR of at least 24 weeks.

In a typical embodiment, the patient treated is a human who has been infected with hepatitis C. In another aspect, the patient, is treated is a mammal infected with hepatitis C such as a simian.

The patients treated according to the described methods may be infected with any of the HCV genotypes 1, 2, 3, 4, 5, and/or 6 (for example, 1a, 1b, 1c, 2a, 2b, 2c, 3a, 3b, 4a, 5a or 6a) (or any combination thereof). In one embodiment, the methods disclosed treat all HCV genotypes (“pan-genotypic treatment”). HCV genotyping can be performed using methods known in the art, for example, VERSANT™ HCV Genotype 2.0 Assay Line Probe Assay (LiPA).

The patients treated according to the present invention may be treatment naïve or treatment-experienced, may be compensated liver patients or decompensated liver patients; cirrhotic or non-cirrhotic; patients with fibrosis (including high levels of fibrosis); any ethnicity; co-infected with another viral infection, for example, HIV infection; a liver transplant patients, or a patient with polymorphism such as Q80K, etc; or an IL28 status patient.

As used herein, “treatment naïve” refers to the patient not having previously received treatment with any drug—investigational or approved—for HCV infection. As used herein, “treatment experienced” refers to a patient who has had at least one previous course of another anti-HCV agent, for example, a non-direct-acting antiviral agent (“DAA”), interferon-based HCV therapy, with or without ribavirin. In some embodiments, the last dose in this previous course occurred at least two months prior to implementing a treatment regime according to the present disclosure.

In some embodiments, the patients treated according to the described methods do not have decompensated liver disease, in which case, the administration in some embodiments terminates after a period of time that is 6 weeks or, in other embodiments, less than 6 weeks, for example, 5, or 4 weeks, and in still other embodiments, the administration terminates after a period of time that is 4 weeks.

In some embodiments, the patients treated according to the described methods are treatment naïve (either with or without decompensated liver disease). In other embodiments, the patients treated are treatment-experienced (either with or without decompensated liver disease). When it is indicated that the patient has decompensated liver disease, it means e.g., that liver function is insufficient, Child-Pugh A, Child-Pugh B, prior to initiation of the treatment, in which case, the administration in some embodiments terminates after a period of time that is 4 weeks, 5 weeks, 6 weeks, 7 weeks or 8 weeks.

Some embodiments of the treatments disclosed herein include the administration of the Compounds (I), (II), and (III), or pharmaceutically acceptable salt, hydrate, or solvate form(s) thereof, and does not include administering interferon, for example, PEGylated interferon, during the treatment period.

In some embodiments, the described methods do not include administration of ribavirin during the treatment period. In other embodiments, the described methods do include administration of ribavirin during the treatment period.

Prior to initiation of treatment, the HCV infection can be diagnosed using methods known in the art, for example, by testing an HCV RNA level present in a biological sample taken from the patient, for example, a blood, plasma, or liver biopsy sample. Patients who may typically be treated using the described methods will have a quantifiable HCV RNA level greater than the lower limit of quantification (“LLOQ”) of the Roche COBAS Ampliprep/COBAS Taqman™ HCV Quantitative Test v2.0 (Roche Diagnostics, Indianapolis, Ind.). The current LLOQ of that assay is 15 IU/mL.

The methods described herein may be used to treat HCV infections that are comorbid with other liver diseases. For example, the HCV infection can be comorbid with liver fibrosis, cirrhosis, Child-Pugh A (mild hepatic impairment), or Child-Pugh B (moderate hepatic impairment), prior to initiation of the treatment. For example, a patient suffering from liver fibrosis may be characterized by methods known in the art, such as a FibroSURE™ score of less than or equal to 0.48 and an aspartate aminotransferase to platelet ratio index (APRI) score of less than or equal to 1.

Patients who can be treated according to the methods of the disclosure, in addition to having an HCV infection prior to initiation of the treatment, can also suffer from cirrhosis prior to initiation of the treatment. For example, a patient can also suffer from cirrhosis characterized by methods known in the art, such as a FibroSURE™ score of greater than 0.75 and an aspartate aminotransferase to platelet ratio index (APRI) score of greater than 2, prior to initiation of the treatment. Alternatively, the patient can also suffer from cirrhosis characterized by a METAVIR score F4, prior to initiation of the treatment.

Patients who can be treated according to the methods of the disclosure, in addition to having an HCV infection prior to initiation of the treatment, can also suffer from Child-Pugh A (mild hepatic impairment) prior to initiation of the treatment.

Patients who can be treated according to the methods of the disclosure, in addition to having an HCV infection prior to initiation of the treatment, can also suffer from Child-Pugh B (moderate hepatic impairment) prior to initiation of the treatment. Evidence of portal hypertension characterized by, for example, esophageal varices or hepatic venous pressure gradient (HVPG) greater than or equal to 10 mm Hg, can be present prior to initiation of the treatment.

An effective amount of a pharmaceutical composition/combination of the disclosure may optionally be an amount sufficient to (a) inhibit the progression of hepatitis C or other disorder described herein; (b) cause a regression of the hepatitis C infection or other disorder described herein; or (c) cause a cure of a hepatitis C infection, or other disorder described herein, for example such that HCV virus or HCV antibodies can no longer be detected in a previously infected patient's blood or plasma.

In certain embodiments, an effective amount of one of the anti-HCV drug combination described herein, optionally in a pharmaceutically acceptable carrier can be used to treat a secondary condition associated with a disorder described herein, for example hepatitis C, including but not limited to those disorders described below in (i) through (viii).

(i) Cryoglobulinemia which is abnormal antibodies (called cryoglobulins) that come from hepatitis C virus stimulation of lymphocytes. These antibodies can deposit in small blood vessels, thereby causing inflammation of the vessels (vasculitis) in tissues throughout the body including the skin, joints and kidneys (glomerulonephritis).

(ii) B-cell non-Hodgkin's lymphoma associated with hepatitis C, which is considered to be caused by excessive stimulation by hepatitis C virus of B-lymphocytes, resulting in abnormal reproduction of the lymphocytes.

(iii) Skin conditions such as lichen planus and porphyria cutanea tarda have been associated with hepatitis C infection.

(iv) Cirrhosis, which is a disease in which normal liver cells are replaced with scar or abnormal tissue. Hepatitis C is one of the most common causes of liver cirrhosis.

(v) Ascites, which is the accumulation of fluid in the abdominal cavity commonly caused by cirrhosis of the liver, which can be caused by hepatitis C infection.

(vi) Hepatocellular carcinoma, of which 50% of the cases in the U.S. are currently caused by chronic hepatitis C infection.

(vii) Hepatitis C related jaundice, which is a yellowish pigmentation caused by increased bilirubin.

(viii) Thrombocytopenia is often found in patients with hepatitis C and may be the result of bone marrow inhibition, decrease in liver thrombopoietin production and/or an autoimmune mechanism. In many patients, as hepatitis C advances, the platelet count decreases and both bone marrow viral inhibition and antiplatelet antibodies increase.

Other symptoms and disorders associated with hepatitis C that may be treated by an effective amount of a pharmaceutical composition/combination of the disclosure include decreased liver function, fatigue, flu-like symptoms: fever, chills, muscle aches, joint pain, and headaches, nausea, aversion to certain foods, unexplained weight loss, psychological disorders including depression, and tenderness in the abdomen.

The active compounds presented herein can also be used to enhance liver function, a problem generally associated with hepatitis C infection, for example, synthetic function including synthesis of proteins such as serum proteins (e.g., albumin, clotting factors, alkaline phosphatase, aminotransferases (e.g., alanine transaminase, aspartate transaminase), 5′-nucleosidase, y glutaminyltranspeptidase, etc.), synthesis of bilirubin, synthesis of cholesterol, and synthesis of bile acids; a liver metabolic function, including carbohydrate metabolism, amino acid and ammonia metabolism, hormone metabolism, and lipid metabolism; detoxification of exogenous drugs; and a hemodynamic function, including splanchnic and portal hemodynamics.

Embodiments of treatment methods according to this invention are envisioned to provide an SVRn of 4, 6, 12, or 24 or greater weeks. Some of these various SVRn are envisioned to apply to at least 80% of the treated patients, in other embodiments they are envisioned to apply to at least 90% of the patients, in other embodiments to at least 95% of the treated patients, while in still other embodiments to more than 95% of the treated patients, and some apply to 100% of the patients. In other embodiments of this invention, various of such SVRn are envisioned to apply to patients infected with HCV genotype 1a containing the NS3 polymorphism Q80K. It is known in the art that patients infected with HCV genotype 1a containing the NS3 polymorphism Q80K demonstrate lower response rates to previously-described treatments, for example, treatments with Simeprevir in combination with PEGylated interferon and ribavirin.

The term “viral relapsers” as used herein is a term known to those skilled in the art and stands for the number of patients, given as an absolute number or as a percentage of the treated patients, who did not achieve SVR12 at the end of the treatment period and have an HCV RNA level of greater than LLOQ during week 24 after the end of the treatment period. Embodiments of treatment methods according to this invention are envisioned to reduce viral relapsers to less than 10% of patients, in other embodiments to less than 5%, while still in other embodiments to less than 2% of patients.

In some embodiments of methods according to this invention, Compounds (I), (II), and (III), or pharmaceutically acceptable salts thereof are administered once per day during the period of administration. In some embodiments, they can be co-administered, in others sequentially administered, while in still others they can be administered substantially simultaneously. IN some embodiments, the drugs are taken in a manner that allows the bioavailabilities to overlap such that the benefit of the combination treatment is achieved. In some of the latter embodiments, administration entails taking such compounds or pharmaceutically acceptable salts thereof within 60, 45 or 30 minutes or less of each other, in some embodiments 15 minutes or less of each other. In some embodiments, the compounds of compounds (I), (II), and (III), or pharmaceutically acceptable salts thereof are administered once per day, at approximately the same time each day. For example, the compounds of compounds (I), (II), and (III), or pharmaceutically acceptable salts thereof are administered within a time range of 4 hours of the original time of administration on the first day, that is, ±2 hours, or ±1 hour, or in still other embodiments±30 minutes of the time on the original administration day.

“Liver function” refers to a normal function of the liver, including, but not limited to, a synthetic function including synthesis of proteins such as serum proteins (e.g., albumin, clotting factors, alkaline phosphatase, aminotransferases (e.g., alanine transaminase, aspartate transaminase), 5′-nucleosidase, y glutaminyltranspeptidase, etc.), synthesis of bilirubin, synthesis of cholesterol, and synthesis of bile acids; a liver metabolic function, including carbohydrate metabolism, amino acid and ammonia metabolism, hormone metabolism, and lipid metabolism; detoxification of exogenous drugs; and a hemodynamic function, including splanchnic and portal hemodynamics.

### Fixed Dose Combinations

An aspect of the invention is a fixed dosage combination with an effective amount for a patient, typically a human, of Simeprevir, Odalasvir and Compound III, to treat hepatitis C or another condition described herein, optionally provided as a pharmaceutically acceptable salt, hydrate or solvate in a pharmaceutically acceptable carrier, in any of the dosage amounts or manners described herein.

In one embodiment, the fixed dose combination includes a spray dried solid dispersion of at least one of the Compounds or its pharmaceutically acceptable salt, solvate, or hydrate, and the composition is suitable for oral delivery. In one aspect of this embodiment, the fixed dose combination includes about 100 mg Simeprevir, 25 mg Odalasvir and 800 mg Compound III. In one aspect of this embodiment, the fixed dose combination includes about 100 mg Simeprevir, 20 mg Odalasvir and 800 mg Compound III, wherein at least one of the Compounds (for example Simeprevir and/or Odalasvir) is in a spray dried solid dispersion. In one aspect of this embodiment, the fixed dose combination includes about 100 mg Simeprevir, 12.5 mg Odalasvir and 800 mg Compound III wherein at least one of the Compounds (for example Simeprevir and/or Odalasvir) is in a spray dried solid dispersion. In one aspect of this embodiment, the fixed dose combination includes about 100 mg Simeprevir, 17.5 mg Odalasvir and 800 mg Compound III wherein at least one of the Compounds (for example Simeprevir and/or Odalasvir) is in a spray dried solid dispersion. In one aspect of this embodiment, the fixed dose combination includes about 100 mg Simeprevir, 10 mg Odalasvir and 800 mg Compound III wherein at least one of the Compounds (for example Simeprevir and/or Odalasvir) is in a spray dried solid dispersion. In one aspect of this embodiment, the fixed dose combination includes about 75 or 100 mg Simeprevir, 12.5 mg Odalasvir and 800 mg Compound III wherein at least one of the Compounds (for example Odalasvir) is in a spray dried solid dispersion. In one embodiment, Compound (III) is not provided as a spray dried dispersion in the fixed dosed composition.

In another embodiment, the fixed dose combination is a granulo layered solid dispersion of at least one of the Compounds or its pharmaceutically acceptable salt, solvate, or hydrate, and the composition is suitable for oral delivery. In one aspect of this embodiment, the fixed dose combination is a granulo layered solid dispersion that includes about 100 mg Simeprevir, 25 mg Odalasvir and 800 mg Compound III. In one aspect of this embodiment, the fixed dose combination is a granulo layered solid dispersion that includes about 100 mg Simeprevir, 20 mg Odalasvir and 800 mg Compound III. In one aspect of this embodiment, the fixed dose combination is a is a granulo layered solid dispersion that includes about 100 mg Simeprevir, 12.5 mg Odalasvir and 800 mg Compound III. In one aspect of this embodiment, the fixed dose combination is a is a granulo layered solid dispersion that includes about 100 mg Simeprevir, 17.5 mg Odalasvir and 800 mg Compound III. In one aspect of this embodiment, the fixed dose combination is a granulo layered solid dispersion includes about 100 mg Simeprevir, 10 mg Odalasvir and 800 mg Compound III. In one aspect of this embodiment, the fixed dose combination is a granulo layered solid dispersion that includes about 75 or 100 mg Simeprevir, 12.5 mg Odalasvir and 800 mg Compound III. In one embodiment, Compound (III) is not provided as a spray dried dispersion in the fixed dosed composition.

In certain embodiments, a spray dried dispersion or granulo layered solid dispersion component is prepared using Odalasvir crystalline dihydrate. In other embodiments, the solid dispersion also contains at least one excipient selected from copovidone, poloxamer and HPMC-AS. In one embodiment the poloxamer is Poloxamer 407 or a mixture of poloxamers that may include Poloxamer 407. In one embodiment HPMC-AS is HPMC-AS-L.

In other embodiments, a fixed dose composition prepared from Compounds I, II and III (or two of the three Compounds), or independently its pharmaceutically acceptable salt, hydrate or solvate composition also comprises one or more of the following excipients: a phosphoglyceride; phosphatidylcholine; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohol such as polyethylene glycol (PEG); polyoxyethylene-9-lauryl ether; a surface active fatty acid, such as palmitic acid or oleic acid; fatty acid; fatty acid monoglyceride; fatty acid diglyceride; fatty acid amide; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebroside; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipid; synthetic and/or natural detergent having high surfactant properties; deoxycholate; cyclodextrin; chaotropic salt; ion pairing agent; glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid; pullulan, cellulose, microcrystalline cellulose, silicified microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan, mannitol, sorbitol, xylitol, erythritol, maltitol, and lactitol, a pluronic polymer, polyethylene, polycarbonate (e.g. poly(1,3-dioxan-2one)), polyanhydride (e.g. poly(sebacic anhydride)), polypropylfumerate, polyamide (e.g. polycaprolactam), polyacetal, polyether, polyester (e.g., polylactide, polyglycolide, polylactide-co-glycolide, polycaprolactone, polyhydroxy acid (e.g. poly(((3-hydroxyalkanoate))), poly(orthoester), polycyanoacrylate, polyvinyl alcohol, polyurethane, polyphosphazene, polyacrylate, polymethacrylate, polyurea, polystyrene, and polyamine, polylysine, polylysine-PEG copolymer, and poly(ethyleneimine), poly(ethylene imine)-PEG copolymer, glycerol monocaprylocaprate, propylene glycol, Vitamin E TPGS (also known as d-α-Tocopheryl polyethylene glycol 1000 succinate), gelatin, titanium dioxide, polyvinylpyrrolidone (PVP), hydroxypropyl methyl cellulose (HPMC), hydroxypropyl cellulose (HPC), methyl cellulose (MC), block copolymers of ethylene oxide and propylene oxide (PEO/PPO), polyethyleneglycol (PEG), sodium carboxymethylcellulose (NaCMC), or hydroxypropylmethyl cellulose acetate succinate (HPMCAS).

In other embodiments, a fixed dose composition prepared from Compounds I, II and III (or two of the three Compounds), or independently its pharmaceutically acceptable salt, hydrate or solvate also comprises one or more of the following surfactants: polyoxyethylene glycol, polyoxypropylene glycol, decyl glucoside, lauryl glucoside, octyl glucoside, polyoxyethylene glycol octylphenol, Triton X-100, glycerol alkyl ester, glyceryl laurate, cocamide MEA, cocamide DEA, dodecyldimethylamine oxide, and poloxamers. Examples of poloxamers include, poloxamers 188, 237, 338 and 407. These poloxamers are available under the trade name Pluronic® (available from BASF, Mount Olive, N.J.) and correspond to Pluronic® F-68, F-87, F-108 and F-127, respectively. Poloxamer 188 (corresponding to Pluronic® F-68) is a block copolymer with an average molecular mass of about 7,000 to about 10,000 Da, or about 8,000 to about 9,000 Da, or about 8,400 Da. Poloxamer 237 (corresponding to Pluronic® F-87) is a block copolymer with an average molecular mass of about 6,000 to about 9,000 Da, or about 6,500 to about 8,000 Da, or about 7,700 Da. Poloxamer 338 (corresponding to Pluronic® F-108) is a block copolymer with an average molecular mass of about 12,000 to about 18,000 Da, or about 13,000 to about 15,000 Da, or about 14,600 Da. Poloxamer 407 (corresponding to Pluronic® F-127) is a polyoxyethylene-polyoxypropylene triblock copolymer in a ratio of between about E101 P56 E101 to about E106 P70 E106, or about E101 P56E101, or about E106 P70 E106, with an average molecular mass of about 10,000 to about 15,000 Da, or about 12,000 to about 14,000 Da, or about 12,000 to about 13,000 Da, or about 12,600 Da.

In yet other embodiments, a fixed dose composition prepared from Compounds I, II and III (or two of the three Compounds), or independently its pharmaceutically acceptable salt, hydrate or solvate also comprises one or more of the following surfactants: polyvinyl acetate, cholic acid sodium salt, dioctyl sulfosuccinate sodium, hexadecyltrimethyl ammonium bromide, saponin, sugar esters, Triton X series, sorbitan trioleate, sorbitan mono-oleate, polyoxyethylene (20) sorbitan monolaurate, polyoxyethylene (20) sorbitan monooleate, oleyl polyoxyethylene (2) ether, stearyl polyoxyethylene (2) ether, lauryl polyoxyethylene (4) ether, block copolymers of oxyethylene and oxypropylene, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, cetylpyridinium chloride, benzalkonium chloride, olive oil, glyceryl monolaurate, corn oil, cotton seed oil, and sunflower seed oil.

In alternative embodiments, a fixed dose composition prepared from Compounds I, II and III (or two of the three Compounds), or independently its pharmaceutically acceptable salt, hydrate or solvate is prepared by a process that includes solvent or dry granulation optionally followed by compression or compaction, spray drying, nano-suspension processing, hot melt extrusion, extrusion/spheronization, molding, spheronization, layering (e.g., spray layering suspension or solution), or the like. Examples of such techniques include direct compression, using appropriate punches and dies, for example wherein the punches and dies are fitted to a suitable tableting press; wet granulation using suitable granulating equipment such as a high shear granulator to form wetted particles to be dried into granules; granulation followed by compression using appropriate punches and dies, wherein the punches and dies are fitted to a suitable tableting press; extrusion of a wet mass to form a cylindrical extrudate to be cut into desire lengths or break into lengths under gravity and attrition; extrusion/spheronization where the extrudate is rounded into spherical particles and densified by spheronization; spray layering of a suspension or solution onto an inert core using a technique such as a convention pan or Wurster column; injection or compression molding using suitable molds fitted to a compression unit; and the like.

Exemplary disintegrants include alginic acid, carboxymethylcellulose calcium, carboxymethylcellulose sodium, cross-linked sodium carboxymethylcellulose (sodium croscarmellose), powdered cellulose, chitosan, croscarmellose sodium, crospovidone, guar gum, low substituted hydroxypropyl cellulose, methyl cellulose, microcrystalline cellulose, sodium alginate, sodium starch glycolate, partially pregelatinized starch, pregelatinized starch, starch, sodium carboxymethyl starch, and the like, or a combination thereof.

Exemplary lubricants include calcium stearate, magnesium stearate, glyceryl behenate, glyceryl palmitostearate, hydrogenated castor oil, light mineral oil, sodium lauryl sulfate, magnesium lauryl sulfate, sodium stearyl fumarate, stearic acid, zinc stearate, silicon dioxide, colloidal silicon dioxide, dimethyldichlorosilane treated with silica, talc, or a combination thereof.

The dosage form cores described herein may be coated to result in coated tablets. The dosage from cores can be coated with a functional or non-functional coating, or a combination of functional and non-functional coatings. “Functional coating” includes tablet coatings that modify the release properties of the total composition, for example, a sustained-release or delayed-release coating. “Non-functional coating” includes a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition. A non-functional coating can also mask the taste of the uncoated composition including the active pharmaceutical ingredient. A coating may comprise a light blocking material, a light absorbing material, or a light blocking material and a light absorbing material.

Exemplary polymethacrylates include copolymers of acrylic and methacrylic acid esters, such as a. an aminomethacrylate copolymer USP/NF such as a poly(butyl methacrylate, (2-dimethyl aminoethyl)methacrylate, methyl methacrylate) 1:2:1 (e.g., EUDRAGIT E 100, EUDRAGIT EPO, and EUDRAGIT E 12.5; CAS No. 24938-16-7); b. a poly(methacrylic acid, ethyl acrylate) 1:1 (e.g., EUDRAGIT L30 D-55, EUDRAGIT L100-55, EASTACRYL 30D, KOLLICOAT MAE 30D AND 30DP; CAS No. 25212-88-8); c. a poly(methacrylic acid, methyl methacrylate) 1:1 (e.g., EUDRAGIT L 100, EUDRAGIT L 12.5 and 12.5 P; also known as methacrylic acid copolymer, type A NF; CAS No. 25806-15-1); d. a poly(methacrylic acid, methyl methacrylate) 1:2 (e.g. EUDRAGIT S 100, EUDRAGIT S 12.5 and 12.5P; CAS No. 25086-15-1); e. a poly(methyl acrylate, methyl methacrylate, methacrylic acid) 7:3:1 (e.g. Eudragit FS 30 D; CAS No. 26936-24-3); f a poly(ethyl acrylate, methylmethacrylate, trimethylammonioethyl methacrylate chloride) 1:2:0.2 or 1:2:0.1 (e.g., EUDRAGITS RL 100, RL PO, RL 30 D, RL 12.5, RS 100, RS PO, RS 30 D, or RS 12.5; CAS No. 33434-24-1); g. a poly(ethyl acrylate, methyl methacrylate) 2:1 (e.g. EUDRAGIT NE 30 D, Eudragit NE 40D, Eudragit NM 30D; CAS No. 9010-88-2); and the like, or a combination thereof.

Suitable alkylcelluloses include, for example, methylcellulose, ethylcellulose, and the like, or a combination thereof. Exemplary water based ethylcellulose coatings include AQUACOAT, a 30% dispersion further containing sodium lauryl sulfate and cetyl alcohol, available from FMC, Philadelphia, Pa.; SURELEASE a 25% dispersion further containing a stabilizer or other coating component (e.g., ammonium oleate, dibutyl sebacate, colloidal anhydrous silica, medium chain triglycerides, etc.) available from Colorcon, West Point, Pa.; ethyl cellulose available from Aqualon or Dow Chemical Co (Ethocel), Midland, Mich. Those skilled in the art will appreciate that other cellulosic polymers, including other alkyl cellulosic polymers, can be substituted for part or all of the ethylcellulose.

Other suitable materials that can be used to prepare a functional coating include hydroxypropyl methylcellulose acetate succinate (HPMCAS); cellulose acetate phthalate (CAP); a polyvinylacetate phthalate; neutral or synthetic waxes, fatty alcohols (such as lauryl, myristyl, stearyl, cetyl or specifically cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, or a combination thereof. Suitable waxes include beeswax, glycowax, castor wax, carnauba wax, microcrystalline wax, candelilla, and wax-like substances, e.g., material normally solid at room temperature and having a melting point of from about 30° C. to about 100° C., or a combination thereof.

In other embodiments, a functional coating may include digestible, long chain (e.g., C₈-C₅₀, specifically C₁₂-C₄₀), substituted or unsubstituted hydrocarbons, such as fatty acids, fatty alcohols, glyceryl esters of fatty acids, mineral and vegetable oils, waxes, or a combination thereof. Hydrocarbons having a melting point of between about 25° C. and about 90° C. may be used. Specifically, long chain hydrocarbon materials, fatty (aliphatic) alcohols can be used.

The coatings can optionally contain additional pharmaceutically acceptable excipients such as a plasticizer, a stabilizer, a water-soluble component (e.g. pore formers), an anti-tacking agent (e.g., talc), a surfactant, and the like, or a combination thereof.

A functional coating may include a release-modifying agent, which affects the release properties of the functional coating. The release-modifying agent can, for example, function as a pore-former or a matrix disrupter. The release-modifying agent can be organic or inorganic, and include materials that can be dissolved, extracted or leached from the coating in the environment of use. The release-modifying agent can comprise one or more hydrophilic polymers including cellulose ethers and other cellulosics, such as hydroxypropyl methylcellulose, hydroxypropylcellulose, hydroxyethylcellulose, methyl cellulose, cellulose acetate phthalate, or hydroxypropyl methylcellulose acetate phthalate; povidone; polyvinyl alcohol; an acrylic polymer, such as gastric soluble Eudragit FS 30D, pH sensitive Eudragit L30D 55, L 100, S 100, or L 100-55; or a combination thereof. Other exemplary release-modifying agents include a povidone; a saccharide (e.g., lactose, and the like); a metal stearate; an inorganic salt (e.g., dibasic calcium phosphate, sodium chloride, and the like); a polyethylene glycol (e.g., polyethylene glycol (PEG) 1450, and the like); a sugar alcohol (e.g., sorbitol, mannitol, and the like); an alkali alkyl sulfate (e.g., sodium lauryl sulfate); a polyoxyethylene sorbitan fatty acid ester (e.g., polysorbate); or a combination thereof. Exemplary matrix disrupters include water insoluble organic or inorganic material. Organic polymers including but not limited to cellulose, cellulose ethers such as ethylcellulose, cellulose esters such as cellulose acetate, cellulose acetate butyrate and cellulose acetate propionate; and starch can function as matrix disrupters. Examples or inorganic disrupters include many calcium salts such as mono-, di- and tri calcium phosphate; silica and, talc.

The coating may optionally contain a plasticizer to improve the physical properties of the coating. For example, because ethylcellulose has a relatively high glass transition temperature and does not form flexible films under normal coating conditions, it may be advantageous to add plasticizer to the ethylcellulose before using the same as a coating material. Generally, the amount of plasticizer included in a coating solution is based on the concentration of the polymer, e.g., can be from about 1% to about 200% depending on the polymer but is most often from about 1 wt % to about 100 wt % of the polymer. Concentrations of the plasticizer, however, can be determined by routine experimentation.

Examples of plasticizers for ethylcellulose and other celluloses include plasticizers such as dibutyl sebacate, diethyl phthalate, triethyl citrate, tributyl citrate, triacetin, or a combination thereof, although it is possible that other water-insoluble plasticizers (such as acetylated monoglycerides, phthalate esters, castor oil, etc.) can be used.

Examples of plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, or a combination thereof, although it is possible that other plasticizers (such as acetylated monoglycerides, phthalate esters, castor oil, etc.) can be used.

Suitable methods can be used to apply the coating material to the surface of the dosage form cores. Processes such as simple or complex coacervation, interfacial polymerization, liquid drying, thermal and ionic gelation, spray drying, spray chilling, fluidized bed coating, pan coating, or electrostatic deposition may be used.

In certain embodiments, an optional intermediate coating is used between the dosage form core and an exterior coating. Such an intermediate coating can be used to protect the active agent or other component of the core subunit from the material used in the exterior coating or to provide other properties. Exemplary intermediate coatings typically include water-soluble film forming polymers. Such intermediate coatings may include film forming polymers such as hydroxyethyl cellulose, hydroxypropyl cellulose, gelatin, hydroxypropyl methylcellulose, polyethylene glycol, polyethylene oxide, and the like, or a combination thereof; and a plasticizer. Plasticizers can be used to reduce brittleness and increase tensile strength and elasticity. Exemplary plasticizers include polyethylene glycol propylene glycol and glycerin.

### EXAMPLES

The following examples are merely illustrative and are not intended to limit the disclosure to the materials, conditions, or process parameters set forth therein.

### Example 1. Drug-Drug Interaction Study in Humans

A drug-drug interaction study (DDI) study is a study designed to investigate whether a drug alters the pharmacokinetics of another drug or drugs or their metabolites. In Example 1, a Phase-1, open-label, two-group, fixed-sequence study was carried out to evaluate the Odalasvir, Simeprevir and Compound (III) combination pharmacokinetics (PK) in healthy volunteers (male or female 18-60 years of age, BMI 18-32 kg/m², minimum weight 50 kg and in good health based on findings of a medical evaluation including medical history, physical examination, laboratory tests and ECG).

Group One

Compound (III)—Subjects received 800 mg of Compound (III) once daily from Days 1-3, Days 11-13, and Days 21-23. PK blood samples for determination of Compound (III) and metabolite concentrations were collected in reference to the Day 3, Day 13, and Day 23 doses.

Simeprevir—Subjects received 150 mg of Simeprevir (SMV) once daily from Days 4-23. PK blood samples for determination of SMV concentrations were collected in reference to the Day 10, Day 13, Day 20, and Day 23 doses.

Odalasvir—Subjects received a loading dose of 150 mg on Day 14, and 50 mg of Odalasvir once daily from Days 15-23. PK blood samples for determination of Odalasvir concentrations were collected in reference to the Day 20 and Day 23 doses.

Group Two

Compound (III)—Subjects received 800 mg of Compound (III) once daily from Days 1-3, Days 11-13 and Days 21-23. PK blood samples for determination of Compound (III) and metabolite concentrations were collected in reference to the Day 3, Day 13, and Day 23 doses.

Simeprevir—subjects receive 150 mg of SMV once daily from Days 4-23. PK blood samples for determination of SMV concentrations were collected in reference to the Day 20 and Day 23 doses.

Odalasvir—subjects receive a loading dose of 150 mg on Day 4, and 50 mg of Odalasvir once daily from Days 5-23. PK blood samples for determination of Odalasvir concentrations were collected in reference to the Day 10, Day 13, Day 20, and Day 23 doses.

This drug-drug interaction study was intended to assess the safety and tolerability of the described drug combinations at pre-defined time points throughout the study. Study completion/follow-up visit(s) were performed 7 and 28 days after completion of the last study assessment.

A primary objective was to evaluate the effect of multiple oral doses of Odalasvir (alone), Simeprevir (alone) and Odalasvir and Simeprevir, on the multiple oral dose PK of Compound (III) (and certain metabolites thereof, such as the major metabolites in systemic circulation) in healthy volunteers. Other objectives were to evaluate the safety and tolerability of multiple oral doses of Compound (III) when administered alone and in combination with single and multiple oral doses of Odalasvir and/or Simeprevir in healthy volunteers; to determine the potential effect of Compound of Compound (III) and/or Odalasvir on the steady-state PK of Simeprevir in healthy volunteers; to determine the potential effect of Compound of Compound (III) and/or Simeprevir on the steady state PK of Odalasvir in healthy volunteers.

The following PK parameters were estimated for Compound (III) (and certain metabolites thereof, such as the major metabolites in systemic circulation), Simeprevir and Odalasvir: (i) maximum observed plasma concentration (Cₘₐₓ); (ii) area under plasma concentration-time curve from time 0 to dosing interval (tau) (AUC₀₋ₜ). Various other PK parameters were estimated including: Cₗₐₛₜ, t_{1/2}, Tₘₐₓ, Tₗₐₛₜ, CL/F, V_{z}/F, and λ_{z}.

Dose Regime

The following daily doses were employed:

### Compound (III): 800 mg

Odalasvir: 150 mg loading dose; 50 mg for the remainder of the study period.

### Simeprevir: 150 mg.

The drug-drug interaction study investigated whether the pharmacokinetics of the compounds individually were altered when administered in combination. Individual pharmacokinetic parameters of the Compounds used for comparison were:
The CYP inhibition potential of SMV is limited (on any CYP).
SMV displayed no induction of CYP3A4 or of CYP1A2, up to 10 μM.
Odalasvir displayed no inhibition of any CYP (up to 10 μM), except for a limited effect on CYP2C8.
Odalasvir displayed no inhibition of CYP3A4/5, 2B6 and 1A2 up to 10 μM.
Compound (III) displayed limited CYP inhibition potential (Compound (III): IC₅₀>26 μM; Compound A-3: IC₅₀>48 μM; Compound A-1: IC₅₀>40 μM).

The following effects of each of SMV, Compound (III) and Odalasvir individually were observed as follows:
SMV in a prior drug-drug interaction study with Digoxin (a pgp-substrate) led to a 35% increase in exposure of Digoxin (see the in vitro inhibition results of SMV below).
SMV in a prior drug-drug interaction study with Rosuvastatin (substrate of OATPs & BCRP) led to a 3-fold increase in Rosuvastatin exposure.

The following effects of each of SMV, Compound (III) and Odalasvir individually on in vitro inhibition of efflux transporter and uptake transporter were also known:
SMV displays strong/moderate in vitro inhibition of the following efflux transporters: P-gp, MRP2 (IC₅₀=6-19 μM), BSEP (IC₅₀=1.7 μM).
Compound (III) displays no in vitro inhibition of the following efflux transporters: Pgp or BCRP
Odalasvir displays the following effects in the listed efflux transporters: moderate inhibition of Pgp (IC₅₀=9.5 μM); no inhibition of MRP2/3, BCRP, or BSEP.
SMV displays in the following uptake transporters: OATP1B1 strong in vitro inhibition (IC₅₀=0.3 μM); other OATPs NTCP (IC₅₀=2.2 μM).
Compound (III) displays no in vitro inhibition of OATP1B1 or OATP1B3.
Odalasvir displays no inhibition of OATP1B1/B3

Herein, “OATP” refers to “organic anion-transporting polypeptide” and “Pgp” refers to P-glycoprotein.

In contrast to the above individual pharmacokinetic parameters of Compounds I-III, the drug-drug interaction studies of the combination of Compounds I-III indicated that the pharmacokinetic parameters of the combination varied and improved unexpectedly. Results are shown in Tables 1-3, below, and in FIGS. 1-5.

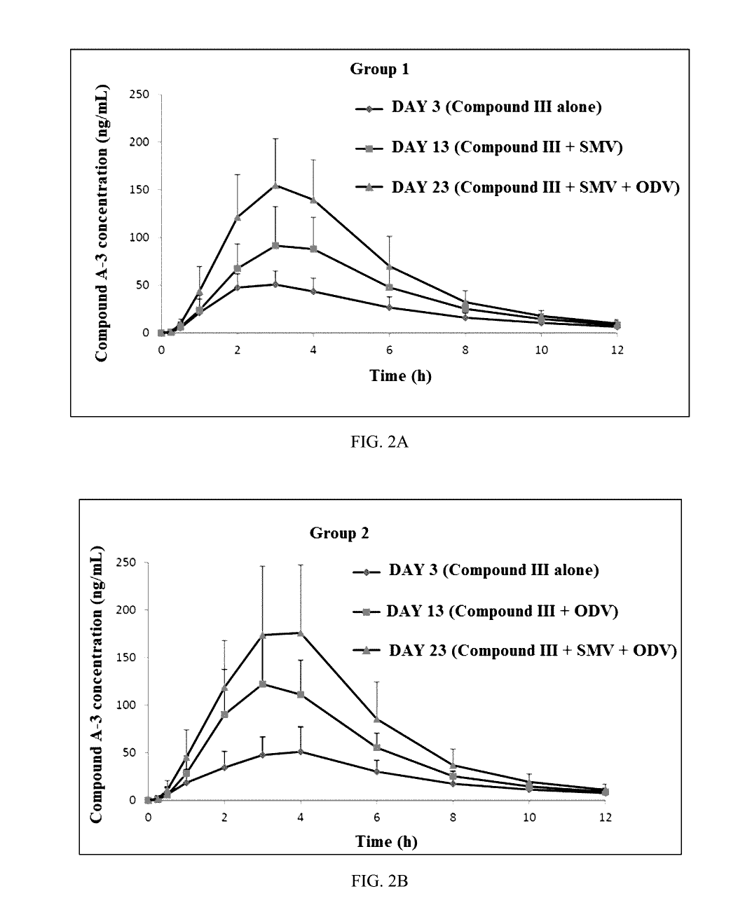

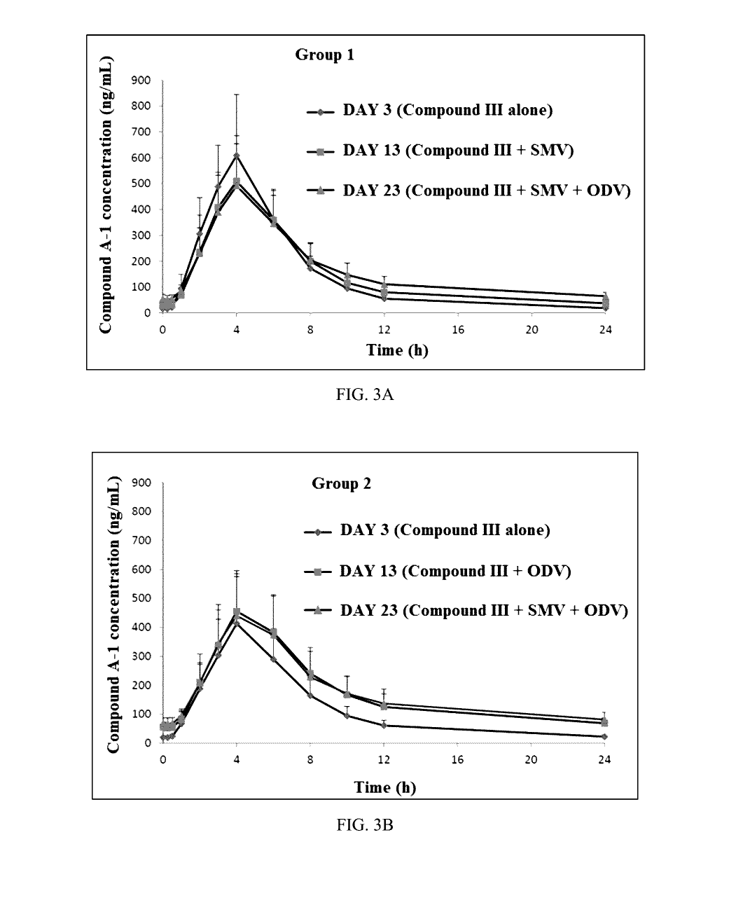

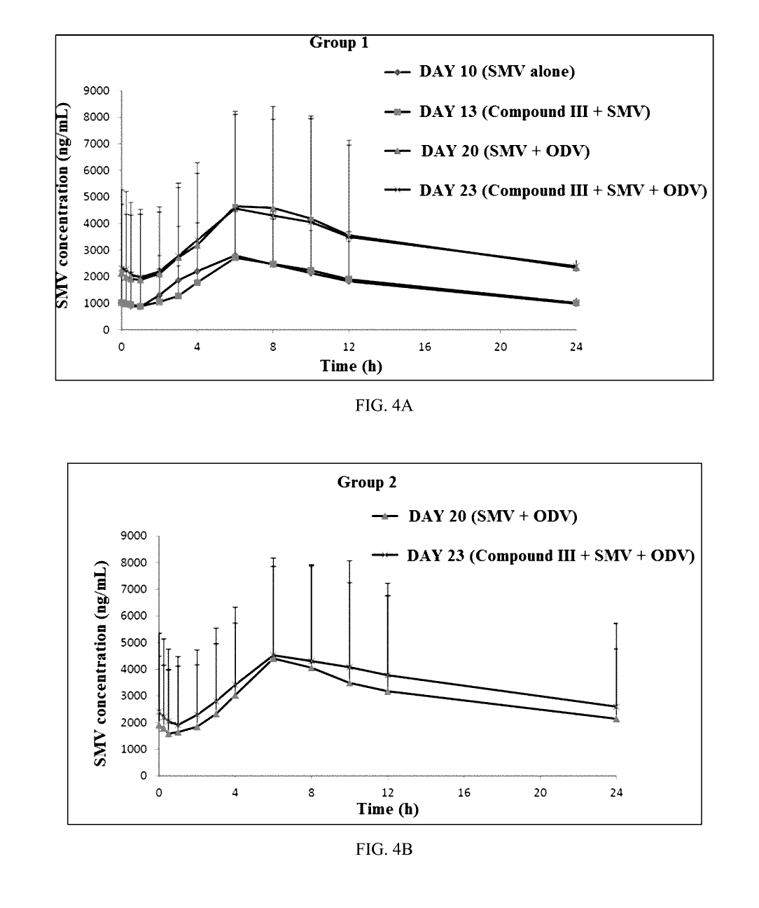

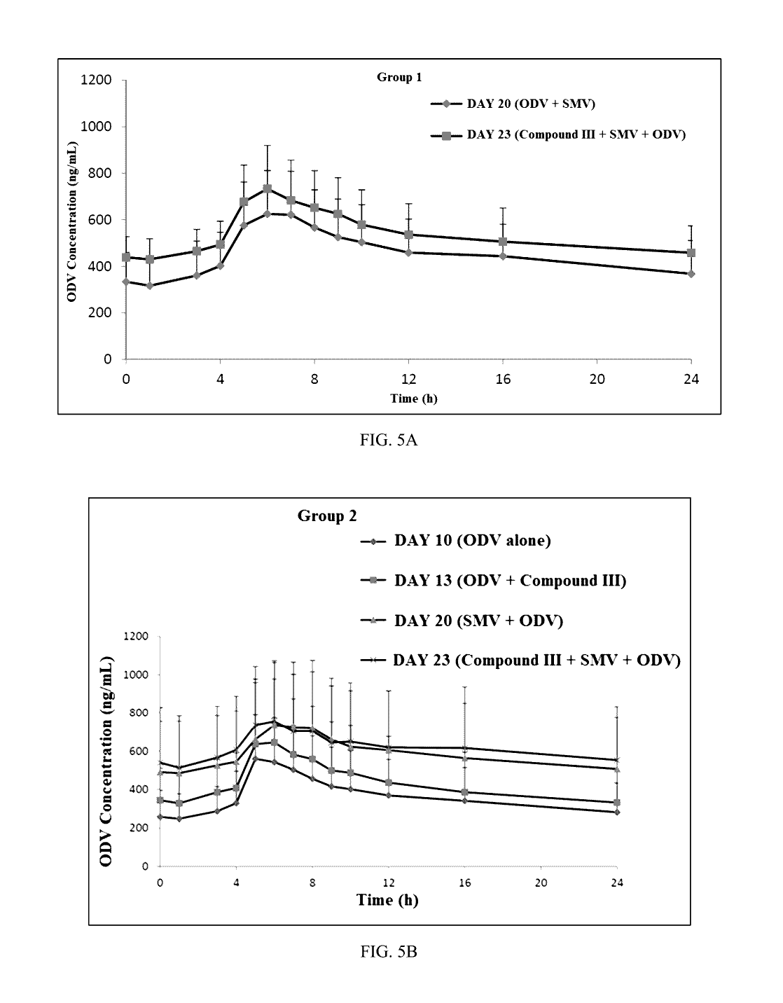

FIG. 1A is a graph showing the concentration of Compound (III) on the y-axis measured in nanograms/milliliter in blood and the x-axis is shown as time in hours after human administration in vivo. FIG. 1B is a graph showing the concentration of Compound (III) on the y-axis measured in nanograms/milliliter in blood and the x-axis is time in hours after human administration. FIG. 2A is a graph showing the concentration of Compound A-3 on the y-axis measured in nanograms/milliliter in blood and the x-axis is time in hours after human administration. FIG. 2B is a graph showing the concentration of Compound A-3 on the y-axis measured in nanograms/milliliter in blood and the x-axis is time in hours after human administration. FIG. 3A is a graph showing the concentration of Compound A-1 on the y-axis measured in nanograms/milliliter in blood and the x-axis is time in hours after human administration. FIG. 3B is a graph showing the concentration of Compound A-1 on the y-axis measured in nanograms/milliliter in blood and the x-axis is time in hours after human administration. FIG. 4A is a graph showing the concentration of SMV on the y-axis measured in nanograms/milliliter in blood and the x-axis is time in hours after human administration. FIG. 4B is a graph showing the concentration of SMV on the y-axis measured in nanograms/milliliter in blood and the x-axis is time in hours after human administration. FIG. 5A is a graph showing the concentration of ODV on the y-axis measured in nanograms/milliliter in blood and the x-axis is time in hours after human administration. FIG. 5B is a graph showing the concentration of ODV on the y-axis measured in nanograms/milliliter and the x-axis is time in hours after human administration.

TABLE 1
Effect of Simeprevir (SMV) and/or Odalasvir on Compound (III)

| | | Compound (III) | | Compound A-3 | | Compound A-1 | |
|---|---|---|---|---|---|---|---|
| | | Cₘₐₓ | AUC₀₋₂₄ | Cₘₐₓ | AUC₀₋₂₄ | Cₘₐₓ | AUC₀₋₂₄ |
| Group | Day | (ng/mL) | (ng * h/mL) | (ng/mL) | (ng * h/mL) | (ng/mL) | (ng * h/mL) |
| Group 1 | Day 3 | 161.6 | 291.6 | 57.1 | 313.4 | 614.2 | 3554 |
| | Compound (III) | | | | | | |
| | alone | | | | | | |
| | Day 13 | 376.2 | 1002 | 102.6 | 521.3 | 523.7 | 3621 |
| | (Compound (III) + | | | | | | |
| | SMV) | | | | | | |
| | Day 23 | 746.6 | 2086 | 162.5 | 822.4 | 499.5 | 4027 |
| | (Compound (III) + | | | | | | |
| | SMV + ODV) | | | | | | |
| Group 2 | Day 3 | 150.7 | 325.4 | 58.0 | 324.7 | 416.3 | 2826 |
| | (Compound (III) | | | | | | |
| | alone) | | | | | | |
| | Day 13 | 515.9 | 1302 | 130.3 | 614.3 | 470.7 | 4196 |
| | (Compound (III) + | | | | | | |
| | SMV) | | | | | | |
| | Day 23 | 949.6 | 2913 | 194.2 | 925.4 | 447.7 | 4321 |
| | (Compound (III) + | | | | | | |
| | SMV + ODV) | | | | | | |

TABLE 2
Effect of Compound (III) and/or Odalasvir on SMV

| | | SMV | |
|---|---|---|---|
| | | | AUC₀₋₂₄ |
| | | Cₘₐₓ | (ng * h/ |
| Group | Day | (ng/mL) | mL) |
| Group 1 | Day 10 (SMV alone) | 2957 | 41258 |
| | Day 13 (SMV + Compound (III)) | 2764 | 40646 |
| | Day 20 (SMV + ODV) | 5219 | 78246 |
| | Day 23 (SMV + Compound (III) + ODV) | 4741 | 77798 |
| Group 2 | Day 20 (SMV + ODV) | 4529 | 70148 |
| | Day 23 (SMV + Compound (III) + ODV) | 4951 | 81020 |

TABLE 3
Effect of SMV and/or Compound (III) on ODV

| | | ACH-3102 | |
|---|---|---|---|
| | | | AUC₀₋₂₄ |
| | | Cₘₐₓ | (ng * h/ |
| Group | Day | (ng/mL) | mL) |
| Group 1 | Day 20 (ODV + SMV) | 648.9 | 10764 |
| | Day 23 (SMV + Compound (III) + ODV) | 744.4 | 12774 |
| Group 2 | Day 10 (ODV alone) | 581.8 | 8717 |
| | Day 13 (ODV + Compound (III)) | 668.8 | 10240 |
| | Day 20 (ODV + SMV) | 769.7 | 13957 |
| | Day 23 (ODV + Compound (III) + SMV) | 780.2 | 14770 |

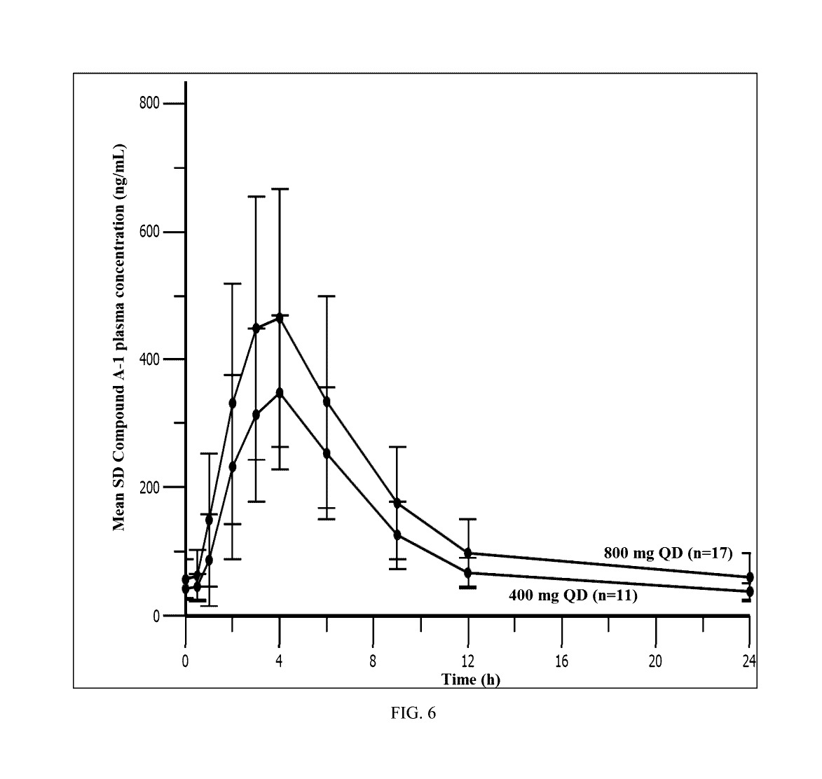

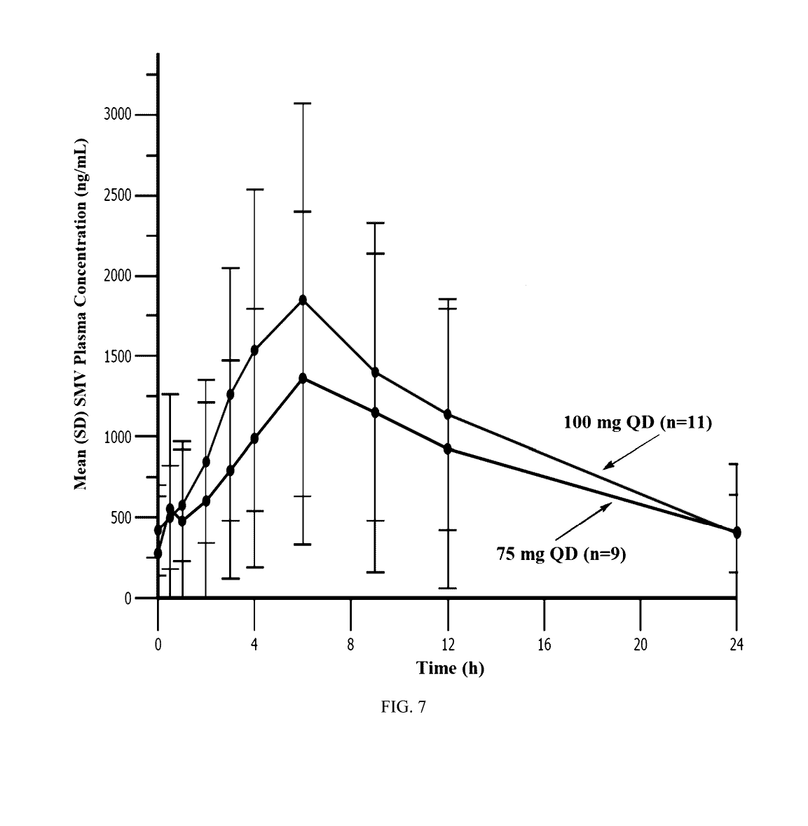

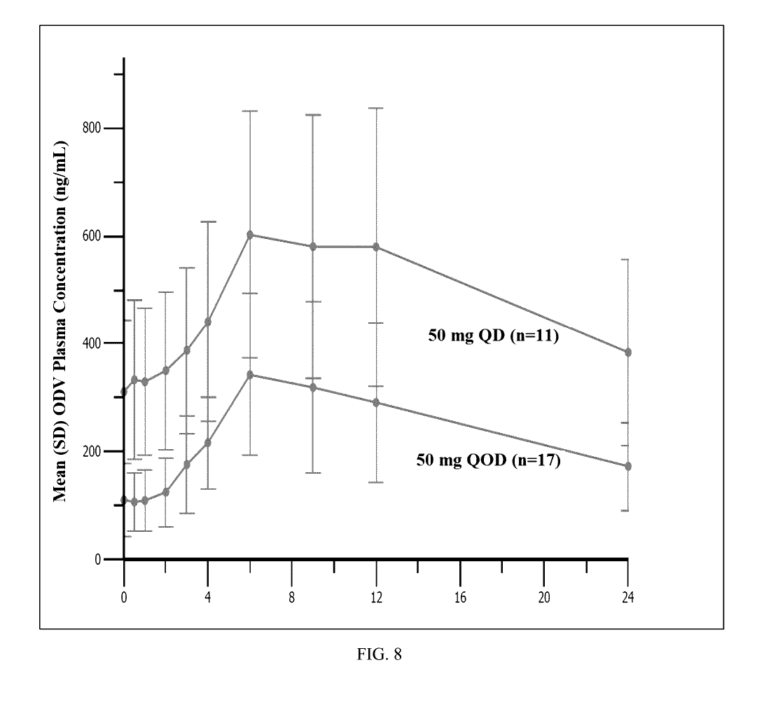

FIGS. 6, 7 and 8 are graphs illustrating pharmacokinetic data obtained in this study. FIG. 6 is a graph of the Compound A1 plasma concentration resulting from once daily administration of single oral doses of Compound (III), SMV, and ODV as a function of time. The y-axis is plasma concentration measured in ng/mL and the x-axis is time measured in hours. The top curve is the plasma concentration curve obtained when Compound (III) is dosed at 800 mg. The bottom curve is the plasma concentration curve obtained when Compound (III) is dosed at 400 mg. FIG. 7 is a graph of the Simeprevir (SMV) plasma concentration resulting from once daily administration of single oral doses of Compound (III), SMV, and ODV as a function of time. The y-axis is plasma concentration measured in ng/mL and the x-axis is time measured in hours. The top curve is the plasma concentration curve obtained when Simeprevir is dosed at 100 mg. The bottom curve is the plasma concentration curve obtained when Simeprevir is dosed at 75 mg. FIG. 8 is a graph of the Odalasvir (ODV) plasma concentration resulting from single oral doses of Compound (III), SMV, and ODV as a function of time. The y-axis is plasma concentration measured in ng/mL and the x-axis is time measured in hours. The top curve is the plasma concentration curve obtained when Odalasvir is dosed daily at 50 mg. The bottom curve is the plasma concentration curve obtained when Odalasvir is dosed every other day at 50 mg.

### Results of the DDI Study Include the Following:

Compound (III) had no apparent effect on SMV or ODV.
SMV caused exposure of Compound (III) to increase ×3 (and Compound A-3 exposure to increase ×1.6, but no apparent effect on Compound A-1).
SMV caused exposure of Odalasvir to increase ×1.6.
Odalasvir caused exposure of Compound (III) to increase ×4; Compound A-3 exposure to increase ×2.6 and Compound A-1 exposure to increase ×1.5.
Odalasvir caused exposure of SMV to increase ×1.6.
The effect of the three Compounds caused: (i) exposure of Compound (III) to increase ×7 to ×8; Compound A-3 exposure to increase ×1.9 to ×2.8; Compound A-1 exposure to increase ×1 to ×1.5. The SMV and Odalasvir interaction appears additive and increases Cₘᵢₙ Compound A-1 by ×3 to ×3.5; (ii) exposure of SMV to increase ×1.6; (iii) exposure of Odalasvir to increase ×1.5 and no apparent contribution of Compound (III) to interaction.

Additionally, reference may be made to FIGS. 1-5 (wherein Odalasvir, is also referred to as “ODV”, ACH-3102 or “ACH”).

In some embodiments, and consistent with the results given herein, the combination of SMV, compound of Compound (III) and ACH-3102 was provided at the following doses which are significantly lower than envisaged on the basis of known individual compound properties and behavior:

SMV=100 mg (once daily i.e. QD) or, in an alternative embodiment, 75 mg QD.
ACH-3102=50 mg QD; without a “loading dose”.

### Compound (III)=400 mg QD.

### Example 2. Phase 2a Combination Study

A randomized, Phase 2a, open-label study was carried out to evaluate the safety, pharmacokinetics and efficacy of the combination of Compound (III), Odalasvir and Simeprevir in Genotype 1 treatment-naïve subjects with chronic hepatitis C. Other treatment-naïve subjects included Genotype 2, 3, 4, 5 and 6.

The combination of Compound (III) and Odalasvir, with or without Simeprevir (SMV), resulted in substantial efficacy in treatment naïve genotype (GT) 1 hepatitis C virus (HCV) infected patients.

The aim of the study was to determine the efficacy, pharmacokinetics (PK), and safety of Compound (III)+Odalasvir±SMV in HCV-infected subjects.

This was an open-label study evaluating various dosing regimens of Compound (III)+Odalasvir±SMV for 6-8 weeks in treatment-naïve HCV-infected subjects with varying clinical characteristics (e.g., GT 1 or 3, presence/absence of compensated Child Pugh A cirrhosis). Efficacy, PK and safety evaluations were conducted during and through 24 weeks post dosing. Up to 15 cohorts were enrolled; data was generated from cohorts that have completed dosing.

Results for 80 treatment naïve, GT 1 infected subjects without cirrhosis who have completed dosing are shown in Table 4 below.

TABLE 4
Dosing Regimens of DDI Study

| | Dose | | | Dosing | Number (%) with |
|---|---|---|---|---|---|
| | Compound (III) | ODV | SMV | Duration | undetectable HCV |
| Cohort # | (mg QD) | (mg) | (mg QD) | (weeks) | RNA (EOT or SVR) |
| 1 | 400 | 50 QD | 100 | 8 | 20/20 (100%), SVR24 |
| 2 | 800 | 50 QOD | — | 8 | 18/20 (90%), SVR12 |
| 3 | 800 | 50 QOD | 75 | 8 | 20/20 (100%), SVR12 |
| 4 | 800 | 50 QOD | 75 | 6 | 20/20 (100%), SVR12 |

*Or below the limit of quantitation (N = 2; Cohort 4 only).
QD: every day;
QOD: every other day;
RNA: ribonucleic acid;
SVR: sustained virologic response.

Compound (III)+ODV±SMV was generally safe and well tolerated. The majority of adverse events (AEs) were mild, most commonly headache, fatigue, and upper respiratory tract infection. There was a single serious AE (Mobitz Type 1 2nd degree atrioventricular block in Cohort 1), which was attributed to treatment. This ECG abnormality was not associated with clinical or echocardiological abnormalities and resolved following treatment discontinuation. No clinically significant laboratory abnormalities were observed.

Consistent with prior studies, increasing Compound (III) dose from 400 to 800 mg increased A-1 (parent nucleoside of Compound (III)) exposure less than proportionally. Observed ODV and SMV exposures in Cohort 1 were higher than anticipated. Reducing ODV dosing from QD to QOD decreased ODV exposure proportionally. Reducing SMV dosing from 100 mg to 75 mg QD decreased SMV exposure less than proportionally.

AL-335+ODV+SMV for 6 or 8 weeks was well tolerated and highly effective in non-cirrhotic patients with HCV GT 1 infection. Ongoing cohorts are evaluating this regimen in patients with HCV GT 3 infection and also GT 1 or 3 infected subjects with cirrhosis.

### Example 3. Preparation of Odalasvir Dihydrate

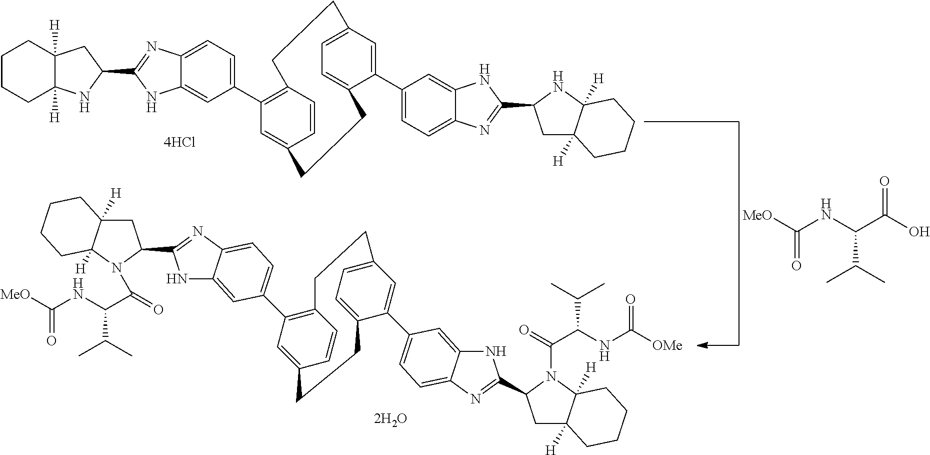

Odalasvir (6,6′-tricyclo[8.2.2.2^{4,7}]hexadeca-1(12),4,6,10,13,15-hexaene-5,11-diylbis[2-[(2S,3aS,7aS)-octahydro-1H-indol-2-yl]-1H-benzimidazole] tetrahydrochloride) can be prepared as described in U.S. Pat. No. 8,809,313 to Wiles et al.

To a solution of Moc-valine methyl ester (0.626 wt. eq.) in dichloromethane was added HOBt (0.56 wt. eq.) followed by EDCI (0.7 wt. eq.). The reaction mixture was cooled to 0° C.-5° C. and 6,6′-tricyclo[8.2.2.2^{4,7}]hexadeca-1(12),4,6,10,13,15-hexaene-5,11-diylbis[2-[(2S,3aS,7aS)-octahydro-1H-indol-2-yl]-1H-benzimidazole] tetrahydrochloride (1 wt. eq.) followed by DIPEA (1.5 vol. eq) were added. The reaction was allowed to warm to room temperature and stirred until completion as analyzed by HPLC. Activated charcoal was added to the reaction mixture and stirring continued for about 30 minutes and filtered over a pad of Celite®. The filtrate was washed with brine containing sodium hydroxide to remove any traces of HOBT. The filtrate was then dried over anhydrous sodium sulfate and evaporated to dryness. Methanol was added to the residue and the mixture heated to about 55° C. and crystalline Odalasvir dihydrate precipitated from the reaction mixture. The solid was filtered to afford the product in about 75% yield.

### Example 4. Preparation of Odalasvir Dihydrate

A round bottomed flask was charged with dichloromethane (10 vol.), N-moc-L-valine (3.0 eq.), and O-(benzotriazol-1-yl)-N,N,N′,N′-tetramethyluronium tetrafluoroborate (TBTU, 3.0 eq.) at 25±5° C. under a nitrogen atmosphere and the reaction was stirred for 5-10 minutes. The reaction was cooled to 0±5° C. under a nitrogen atmosphere and stirred for 5-10 minutes. Odalasvir (1.0 eq.) was added to the reaction at 0±5° C. and stirred for 20-30 minutes under a nitrogen atmosphere. Diisopropylethylamine (7.1 eq.) was slowly added to the reaction through an addition vessel while maintaining the temperature at 0±5° C. over a period of 2 hours under a nitrogen atmosphere. The reaction temperature was raised to 25±5° C. and the reaction was stirred for 24 hours. The reaction was diluted with dichloromethane (10 vol.) and stirred for 10 minutes. Activated charcoal (0.1 w/w) was added at 25±5° C. and stirred for 30-40 minutes. The reaction was filtered through a Celite® bed, the Celite® bed was washed with dichloromethane (5 vol.), and vacuum dried for 20-30 minutes. The organic layer was washed with sodium hydroxide in 13% sodium chloride solution (10 vol.×3). The organic layer was washed with water (10 vol.), diluted with citric acid monohydrate solution (10 vol.×2) and stirred for 1 hour. The organic layer was separated, washed with water (10 vol.), washed with 8% sodium bicarbonate solution (10 vol.) and washed with water (10 vol.). The organic layer was dried over anhydrous sodium sulphate (0.5 w/w), filtered through a Celite® bed and the Celite® bed was washed with dichloromethane (4 vol.). The organic layer was passed through a cartridge filter and the cartridge was washed with dichloromethane (3 vol.). The filtrate was concentrated under vacuum below 55° C. until 1: about 2.0 w/w stage (product:dichloromethane). Methanol (6 vol.) was added at a temperature below 55° C. and the reaction was concentrated at a temperature below 55° C. under vacuum until 1: about 3.0 w/w stage (product:solvent). The reaction was cooled to 25±5° C. and cartridge filtered methanol (15 vol.) was added. The reaction temperature was raised to 65±5° C. and the reaction was stirred for 6 hours. The reaction was cooled to 25±5° C. and stirred for 1 hour. The product was collected, washed with methanol (2 vol.) and spin dried for 20-30 minutes. The purity was not less than 97.0%.

### Example 5. Preparation of Odalasvir Dihydrate

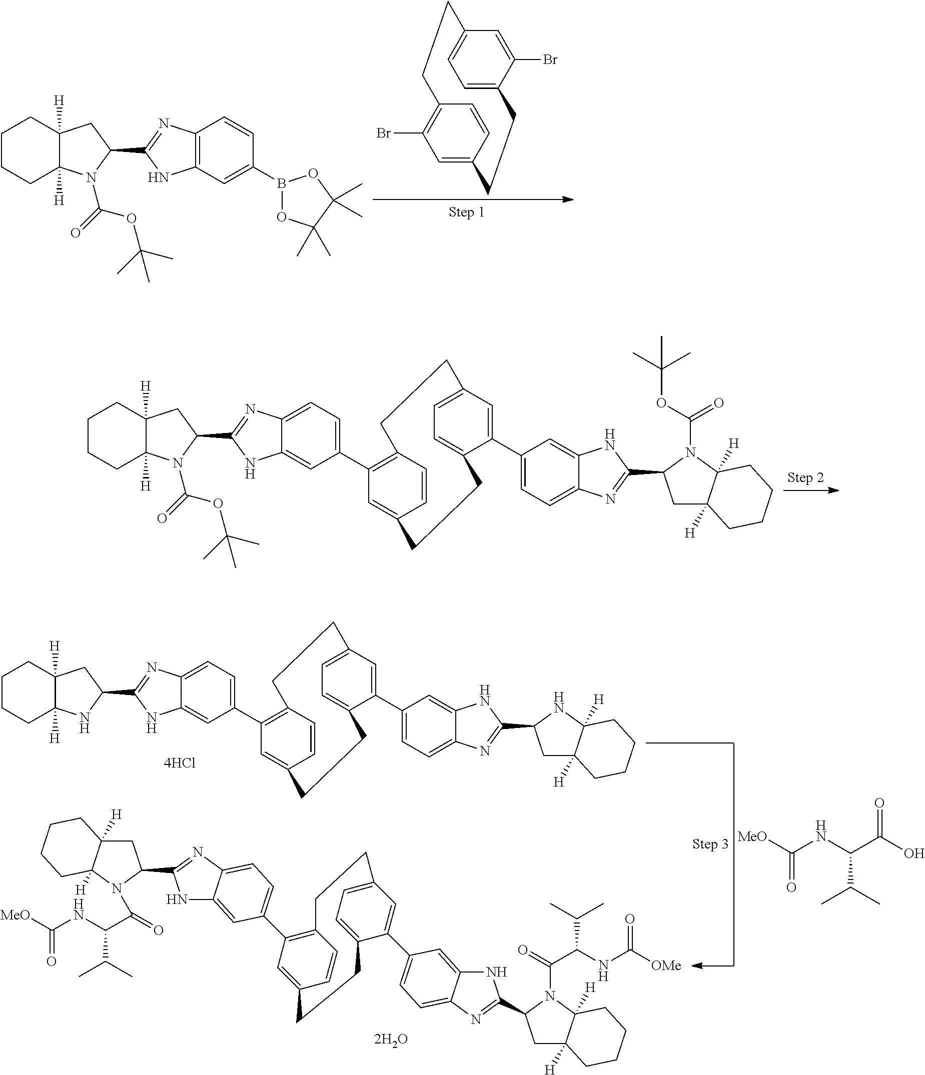

### Step 1: Preparation of di-tert-butyl (2S,3aS,7aS,2′S,3a′S,7a′S)-2,2′-[tricyclo[8.2.2.2^{4,7}]hexadeca-1(12),4,6,10,13,15-hexaene-5,11-diylbis(1H-benzimidazole-6,2-diyl)]bisoctahydro-1H-indole-1-carboxylate

tert-Butyl (2S,3aS,7aS)-2-[6-(4,4,5,5-tetramethyl-1,3,2-dioxaborolan-2-yl)-1H-benzimidazol-2-yl]octahydro-1H-indole-1-carboxylate and pseudo-para-5,11-dibromotricyclo[8.2.2.2^{4,7}]hexadeca-1(12),4,6,10,13,15-hexaene can be prepared as described in U.S. Pat. No. 8,809,313 to Wiles et al.

tert-Butyl (2S,3aS,7aS)-2-[6-(4,4,5,5-tetramethyl-1,3,2-dioxaborolan-2-yl)-1H-benzimidazol-2-yl]octahydro-1H-indole-1-carboxylate was coupled with pseudo-para-5,11-dibromotricyclo[8.2.2.2^{4,7}]hexadeca-1(12),4,6,10,13,15-hexaene in the presence of a palladium catalyst such as Pd(PPh₃)₄ and cesium carbonate in aqueous dimethyl sulfoxide (DMSO) as the solvent. After completion of the reaction, the reaction mixture was added to water, and the precipitated product was isolated and washed with water and acetonitrile. Subsequently, the crude product was dissolved in dichloromethane and the organic layer was separated and washed with water. Then, the dichloromethane was chased out with methanol and acetonitrile, which was followed by the addition of acetonitrile. The resulting precipitate was isolated, washed with acetonitrile, and dissolved in a dichloromethane/methanol mixture. A solvent switch to n-heptane was performed and the crystallized product was isolated, washed with n-heptane, and dried.

### Step 2. Preparation of 6,6′-tricyclo[8.2.2.2^{4,7}]hexadeca-1(12),4,6,10,13,15-hexaene-5,11-diylbis[2-[(2S,3aS,7aS)-octahydro-1H-indol-2-yl]-1H-benzimidazole] tetrahydrochloride

To di-tert-butyl (2S,3aS,7aS,2′S,3a′S,7a′S)-2,2′-[tricyclo[8.2.2.2^{4,7}]hexadeca-1(12),4,6,10,13,15-hexaene-5,11-diylbis(1H-benzimidazole-6,2-diyl)]bisoctahydro-1H-indole-1-carboxylate in dichloromethane and methanol, a solution of hydrogen chloride in 1,4-dioxane was added. After completion of the reaction, a solvent switch to methanol was performed. Subsequently, the precipitate was isolated, washed with methanol, and dried. Optionally, the precipitate was then treated with dichloromethane, isolated, washed with dichloromethane, and dried.

### Step 3. Preparation of the dihydrate of methyl [(2S)-1-[(2S,3aS,7aS)-2-[6-[11-[2-[(2S,3aS,7aS)-1-[(2S)-2-[(methoxy carbonyl)amino]-3-methylbutanoyl]octahydro-1H-indol-2-yl]-1H-benzimidazol-6-yl]tricyclo[8.2.2.2^{4,7}]hexadeca-1 (12),4,6,10,13,15-hexaen-5-yl]-1H-benzimidazol-2-yl]octahydro-1H-indol-1-yl]-3-methyl-1-oxobutan-2-yl]carbamate

To N-(methoxycarbonyl)-L-valine and 1-[bis(dimethylamino)methylene]-1H-benzotriazol-1-ium 3-oxide tetrafluoroborate (TBTU) in dichloromethane, 6,6′-tricyclo[8.2.2.2^{4,7}]hexadeca-1 (12),4,6,10,13,15-hexaene-5,11-diylbis[2-[(2S,3aS,7aS)-octahydro-1H-indol-2-yl]-1H-benzimidazole] tetrahydrochloride was added. Subsequently, N-ethyl-N-isopropylpropan-2-amine (DIPEA) was added slowly to the reaction mixture. After completion of the reaction, dichloromethane was added and the mixture was washed with an aqueous solution of sodium chloride and sodium hydroxide to remove traces of 1H-benzotriazol-1-ol (HOBt) and N-(methoxycarbonyl)-L-valine. Subsequently, the mixture was washed consecutively with water, an aqueous citric acid solution, water, an aqueous sodium hydrogen carbonate solution, and water. After the mixture was partially concentrated, a solvent switch to methanol was performed and the crystallized product was isolated, washed with methanol, and dried. Optionally, the crystallization from methanol may be repeated if required to meet the acceptance criteria, and/or the product may be recrystallized from dichloromethane/methanol, isolated, washed with methanol and dried. The product was obtained as the dihydrate.

### Example 6. Recrystallization of Odalasvir Dihydrate

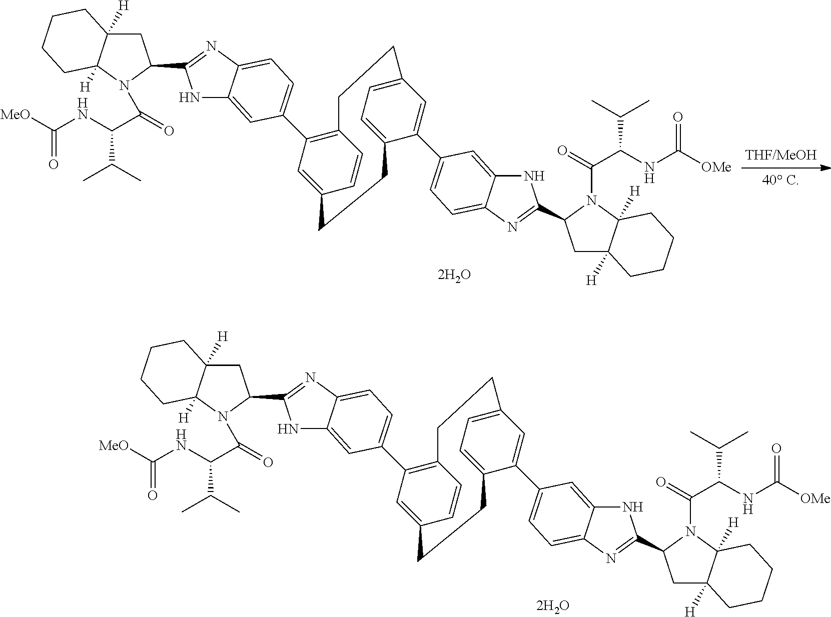

EasyMax laboratory reactors (Mettler Toledo, USA) equipped with 100 mL vessels were charged Odalasvir dihydrate and THF at a ratio of 1 mol Odalasvir dihydrate/2.766 L THF. The reactions were stirred at 250 to 350 rpm using Agitators equipped with four-blade 451 angle impellers and heated to 40° C. for about 20 to 30 minutes or until the Odalasvir dihydrate was dissolved. Samples were taken and analyzed for their water content by the KF method; the water content should be between 0.8-1.5 wt %, and if the water content is lower than 0.8 wt %, water can be added into the solution to reach the required level of water concentration. Methanol (0.9605 L_{MeOH}/Odalasvir dihydrate) was added over a 10 minute period and the reactions were stirred for an additional 5 to 10 minutes. The reactions were seeded with 3 wt % of Odalasvir dihydrate (0.03 1 kg/mol API) at 40° C. and secondary nucleation appeared. The reactions were stirred for an additional 20 minute. Methanol (6.264 L_{MeOH}/mol Odalasvir dihydrate) was added according to Table 5 using a non-linear profile over 2-3 hours.

TABLE 5
Methanol added to Reaction Vessel using a Non-linear
Profile over 2-3 Hours

| | MeOH added | ΔMeOH added | |
|---|---|---|---|
| MeOH added (%) | (L/mol per ODV) | (L/mol per ODV) | Time (hours) |
| 0 | 0 | 0 | 0 |
| 5 | 0.313 | 0.313 | 0.75 |
| 10 | 0.626 | 0.313 | 1.22 |
| 15 | 0.940 | 0.313 | 1.53 |
| 20 | 1.253 | 0.313 | 1.75 |
| 25 | 1.566 | 0.313 | 1.9 |
| 30 | 1.879 | 0.313 | 2.02 |
| 35 | 2.192 | 0.313 | 2.11 |
| 40 | 2.506 | 0.313 | 2.19 |
| 45 | 2.819 | 0.313 | 2.24 |
| 50 | 3.132 | 0.313 | 2.29 |
| 60 | 3.758 | 0.626 | 2.36 |
| 70 | 4.385 | 0.626 | 2.41 |
| 80 | 5.011 | 0.626 | 2.45 |
| 90 | 5.638 | 0.626 | 2.48 |
| 100 | 6.264 | 0.626 | 2.5 |

The suspensions were heated to 60° C. over a 60 minute period. The reactions were cooled to 5° C. over a 120 minute period. The reactions were stirred at 5° C. for 90-120 minutes and filtered at the lab temperature. The products were washed twice with methanol (2.3051 L_{MeOH}/mol API), once with precooled water (2.3051 L_{MeOH}/mol API), and dried at 45-50° C. with a trace of water in the oven for 24 hours. The products were sampled and the solid form was analyzed by XRD. The water content was determined by KF. The residual solvent content of MeOH and THF were determined by GC head space (GCHS). Drying of the product was complete when the residual MeOH concentration was below 50 ppm and the water content was between 2.9-3.7 wt %. (Theoretical yield 93-96%)

### Example 7. Preparation of Fixed-Dose Combinations of Simeprevir/Odalasvir/Compound III

Fixed dosage forms of Simeprevir/Odalasvir/Compound III were prepared as immediate release fixed dose combination tablets for oral administration. Examples of four different tablet formulations were prepared. Three fixed dosage forms (FDC01, FDC02 and FDC04) contained 100 mg Simeprevir, 50 mg Odalasvir and 800 mg Compound III. Another fixed dosage form (FDC03), contained 100 mg Simeprevir, 50 mg Odalasvir and 400 mg Compound III. The fixed dosage forms contained Simeprevir as a spray dried powder (SDP), Odalasvir as a spray dried powder and Compound III.

### Example 8. Preparation of Simeprevir Spray Dried Powder

Methanol and DL-alpha-tocopherol (vitamin D) were mixed. Methanol, methylene chloride, purified water, sodium hydroxide and Simeprevir were mixed and filtered. The two solutions were combined, spray dried, and the product was dried and packaged.

An 8 kg batch of Simeprevir SDP required Simeprevir 7.764 kg; sodium hydroxide 0.414 kg; and DL-alpha-tocopherol (vitamin-E) 0.008 kg. Purified water 2.243 kg; methanol 34.49 kg and methylene chloride 6.734 were used for processing.

The Simeprevir SDP contained an equivalent of 970.56 mg (Simeprevir) free form per gram of the SDP. The Simeprevir SDP was an amorphous sodium salt which also contained an antioxidant DL-alpha-tocopherol. Table 6 lists the composition of Simeprevir eq 970.56 mg/g SDP (SDP44) used to produce the oral tablets FDC01, FDC02, FDC03 and FDC04.

TABLE 6
Composition of the Simeprevir SDP

| | | Quantity per unit |
|---|---|---|
| Component | Function | (mg) |
| Simeprevir | Active | 970.56 |
| Sodium hydroxide | Simeprevir sodium | 51.77 |
| | salt formation | |
| DL-Alpha- | Antioxidant | 1.00 |
| Tocopherol | | |
| Purified water | Process Solvent | 280.39 |
| Methanol^{c} | Process Solvent | 4310.90 |
| Methylene chloride | Process Solvent | 841.69 |
| Total weight | | 1000.00 |

### Example 9. Preparation of SDP Containing an Equivalent of 292.03 mg/g ODV

Acetone was transferred into a suitable container and stirred using a suitable mixer. While stirring, copovidone was added into the container. The mixture was stirred until dissolved. The poloxamers were added to the solution with stirring. The mixture was stirred until dissolved. Odalasvir dihydrate was added with stirring to the solution. The mixture was stirred until dissolved. The mixture was spray dried with spray solution using a suitable spray dryer and the resulting spray dry product was collected in a suitable container. The spray dried product was dried in a suitable dryer. The SDP was collected and packaged in a suitable container.

The Odalasvir spray dried powder contained an equivalent of 292.03 mg Odalasvir free form per gram of the SDP. This SDP was used in FDC01.

To prepare a batch of Odalasvir 292.03 mg/g SDP: Odalasvir 96.81 g; copovidone 161.7 g; poloxamers 64.87 g and acetone 1293 g were used. Table 7 lists the composition of Odalasvir eq. 292.03 mg/g SDP.

### Example 10. Preparation of SDP Containing an Equivalent of 491.16/Mg/g ODV

Acetone was transferred into a suitable container and stirred using a suitable mixer. While stirring, hypromellose acetate succinate was added into the container. The mixture was stirred until dissolved. Odalasvir dihydrate was added with stirring to the solution. The mixture was stirred until dissolved. The mixture was then spray dried with spray solution using a suitable spray dryer and the resulting spray dry product was collected in a suitable container.

The Odalasvir spray dried powder contained an equivalent of 491.16 mg Odalasvir free form per gram of the SDP. This SDP was used in the fixed dosage form FDC02 and FDC03. To prepare the batch of Odalasvir eq 491.16 mg/g SDP: Odalasvir 81.41 g; hypromellose acetate succinate 81.41 g and acetone 1873 g were used. Table 7 lists the composition of composition of Odalasvir eq. 491.16/mg/g SDP.

### Example 11. Preparation of SDP Containing an Equivalent of 243.43 mg/g ODV

Acetone was transferred into a suitable container and stirred using a suitable mixer. While stirring, hypromellose acetate succinate was added into the container. The mixture was stirred until dissolved. Odalasvir dihydrate was added with stirring to the solution. The mixture was stirred until dissolved. The mixture was spray dried with spray solution using a suitable spray dryer and the resulting spray dry product was collected in a suitable container.

The Odalasvir spray dried powder contained an equivalent of 243.43 mg Odalasvir free form per gram of the SDP.

To prepare a batch of Odalasvir eq 243.43 mg/g SDP; Odalasvir 44.13 g; hypromellose acetate succinate 132.4 g and acetone 2766 g are used. Table 7 shows the composition of composition of Odalasvir eq. 243.43 mg/g SDP.

TABLE 7
Composition of Odalasvir Spray Dried Powder

| | | Quantity per | Quantity per | Quantity per |
|---|---|---|---|---|
| | | unit (mg) | unit (mg) | unit (mg) |
| Component | Function | Eq. 292.03 mg/g | Eq 491.16 mg/g | Eq 243.43 mg/g |
| Odalasvir | Active | 302.54 | 508.84 | 252.19 |
| Copovidone | Stabilizer | 505.26 | — | — |
| Poloxamers | Solubilizer | 202.71 | — | — |
| Hypromellose | Stabilizer | — | 508.84 | 756.57 |
| acetate succinate | | | | |
| Acetone | Process Solvent | 4042.04 | 11703.32 | 15803.91 |
| Total weight | Solvent | 1000.00 | 1000.00 | 1000.00 |

### Example 12. Exemplary Process for a Fixed Dose Combination of Simeprevir, Odalasvir, and Compound III

Simeprevir spray dried product, Odalasvir spray dried product, Compound III, croscarmellose sodium and silicified microcrystalline cellulose were blended. Magnesium stearate was added and blended. The product was dry granulated, and screened. Silicified microcrystalline cellulose and croscarmellose were added and blended. Magnesium stearate was added and blended. The product was compressed into tablets and packaged. Table 8 lists the compositions of the tablets FDC01, FDC02, FDC03 and FDC04.

TABLE 8
Composition in Tablets FDC01, FDC02, FDC03 and FDC04

| | | | | | Quantity |
|---|---|---|---|---|---|
| | | Quantity per | Quantity per | Quantity per | per unit |
| | | unit (mg) | unit (mg) | unit (mg) | (mg) |
| | | Eq. 100/eq | Eq. 100/eq | Eq. 100/eq | Eq. 100/eq |
| | | 50/800 mg | 50/800 mg | 50/400 mg | 50/800 mg |
| | | Tablet | Tablet | Tablet | Tablet |
| Component | Function | FDC01 | FDC02 | FDC03 | FDC04 |
| Intragranular | | | | | |
| Phase | | | | | |
| Simeprevir eq | Active | 103.03 | 103.03 | 103.03 | 103.03 |
| 970.56 mg/g SDP | | | | | |
| Odalasvir eq | Active | 171.22 | — | — | — |
| 292.03 mg/g SDP | | | | | |
| Odalasvir eq | Active | — | 101.80 | 101.80 | — |
| 491.16 mg/g SDP | | | | | |
| Odalasvir eq | Active | — | — | — | 205.40 |
| 243.43 mg/g SDP | | | | | |
| Compound III | Active | 800.00 | 800.00 | 400.00 | 800.00 |
| Croscarmellose | Disintegrant | 24.00 | 24.00 | 14.30 | 24.00 |
| sodium | | | | | |
| Silicified | Filler | 437.75 | 507.17 | 293.27 | 403.57 |
| Microcrystalline | | | | | |
| Cellulose | | | | | |
| Magnesium | Lubricant | 8.00 | 8.00 | 4.50 | 8.00 |
| stearate | | | | | |
| Extragranular | | | | | |
| Phase | | | | | |
| Croscarmellose | Disintegrant | 24.00 | 24.00 | 14.30 | 24.00 |
| Sodium | | | | | |
| Silicified | Filler | 24.00 | 24.00 | 14.30 | 24.00 |
| Microcrystalline | | | | | |
| Cellulose | | | | | |
| Magnesium | Lubricant | 8.00 | 8.00 | 4.50 | 8.00 |
| Stearate | | | | | |
| Core Tablet | | 1600.00 | 1600.00 | 950 | 1600.00 |
| Weight | | | | | |

### Example 13. PK Analysis of Compounds and Metabolites from Combination Study

SMV (100 mg), ODV (50 mg), and Compound (III) (800 mg) were administered as three different formulations or together as single doses in vivo to healthy volunteers. The arms of the study included:
Arm 1 (Reference Arm): Single oral doses of single agents, one SMV 100-mg capsule, one ODV 50-mg tablet as granule layered solid dispersion with copovidone and poloxamers, and two Compound (III) 400-mg tablets, administered together.
Arm 2: Single oral dose of fixed dose combination (FDC02): SMV 100 mg+ODV 50 mg as a spray dried solid dispersion with HPMC-AS L in a ratio ODV/HPMC-AS L 1/1 w/w+Compound (III) 800 mg
Arm 3: Single oral dose of fixed dose combination (FDC04): SMV 100 mg+ODV 50 mg as a spray dried solid dispersion with HPMC-AS L in a ratio ODV/HPMC-AS L 1/3 w/w+Compound (III) 800 mg
Arm 4: Single oral dose of fixed dose combination (FDC01): SMV 100 mg+ODV 50 mg as a spray dried solid dispersion with copovidone and poloxamers+Compound (III) 800 mg

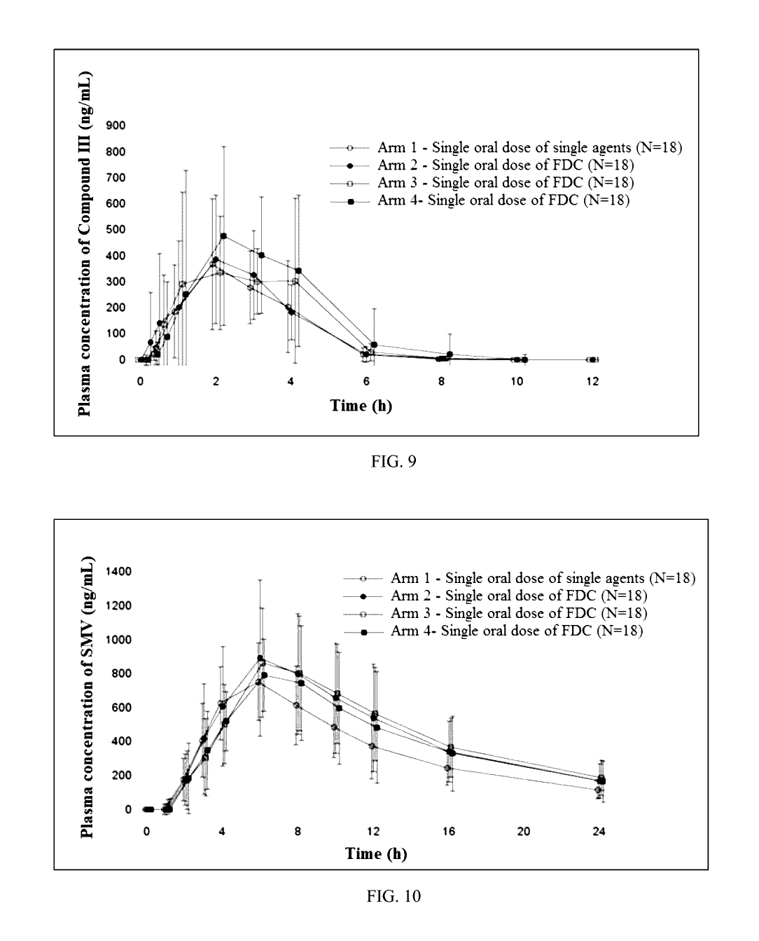

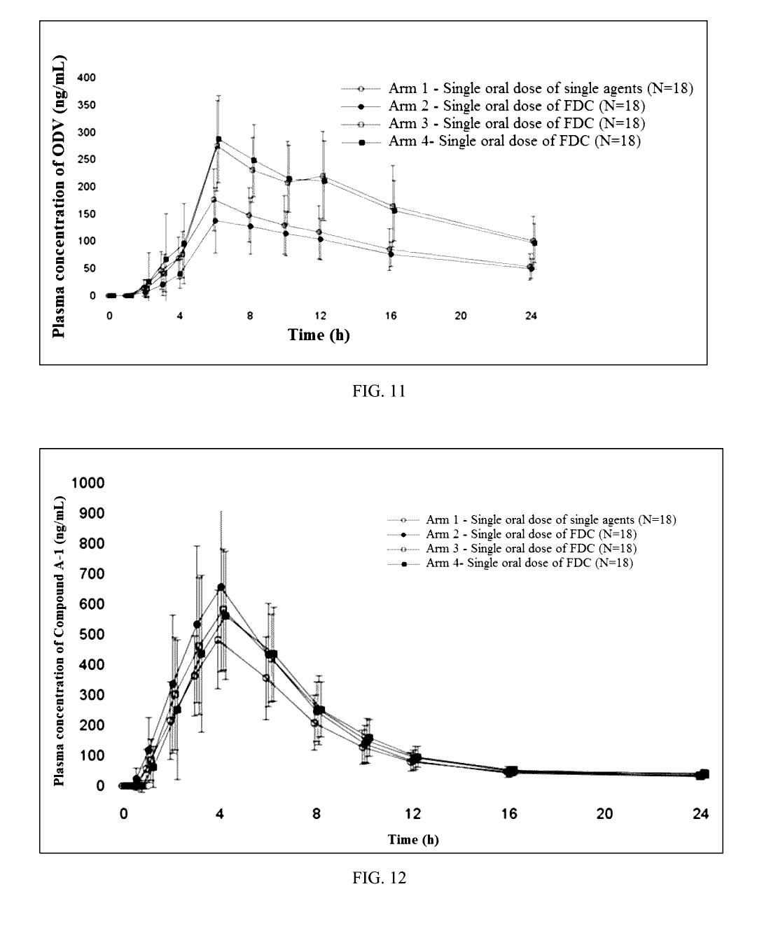

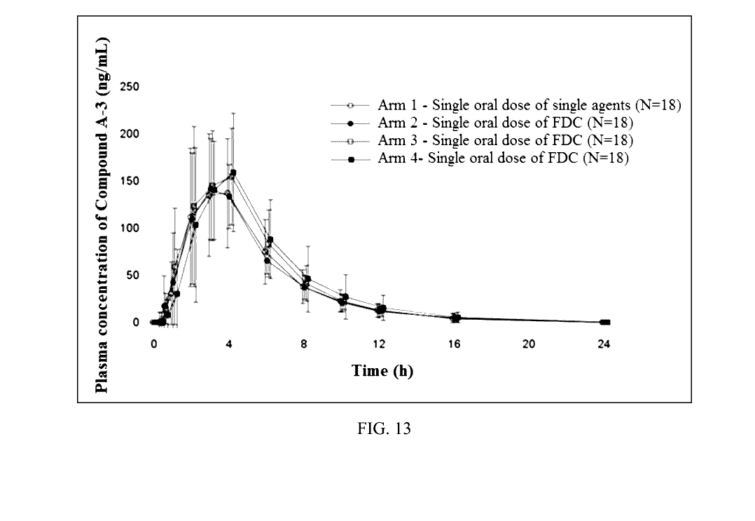

PK parameters, including Cₘₐₓ, tₘₐₓ, AUCₗₐₛₜ, and AUC_{∞} were measured for Compound (III), ODV, SMV, and metabolites Compound A-I and Compound A-3 for each Arm of the study. Arms that included fixed dose combinations (FDC) (Arm 2, Arm 3, and Arm 4) were then compared to a single oral dose (Arm 1, the reference Arm). Table 9, Table 10, Table 11, Table 12 and Table 13 display relevant PK parameters and FIG. 9, FIG. 10, FIG. 11, FIG. 12, and FIG. 13 depict the plasma concentration over time of SMV, ODV, Compound A-1, and Compound A-3 for each Arm of the study.

The plasma concentration of Compound (III) over 12 hours was measured for each Arm of the study. The results are illustrated in FIG. 9, wherein the Compound (III) plasma concentration is shown on the y-axis (measured in ng/mL) and time is measured in hours on the x-axis. SMV, ODV, and Compound (III) were administered as single agents in the combination therapy (Arm 1) or a fixed dose combination (FDC) (Arm 2, Arm 3, and Arm 4). The curve with the open circles is the concentration curve obtained when ODV, SMV, and Compound III are administered together as single agents (Arm 1). The curve with the closed circles is the concentration curve when ODV, SMV, and Compound III are administered as a FDC: SMV 100 mg+ODV 50 mg as a spray dried solid dispersion with HPMC-AS L in a ratio ODV/HPMC-AS L 1/1 w/w+Compound III 800 mg (Arm 2). The curve with the open squares is the concentration curve when ODV, SMV, and Compound III are administered as a FDC: SMV 100 mg+ODV 50 mg as a spray dried solid dispersion with HPMC-AS L in a ratio ODV/HPMC-AS L 1/3 w/w+Compound III 800 mg (Arm 3). The curve with the closed squares is the concentration curve when ODV, SMV, and Compound III are administered as a FDC: SMV 100 mg+ODV 50 mg as a spray dried solid dispersion with copovidone and poloxamers+Compound III 800 mg (Arm 4).

Table 9 reports the Cₘₐₓ, tₘₐₓ, AUCₗₐₛₜ, AUC_{∞}, and t_{1/2term} for Compound (III) for each Arm of the study. The least square means ratio comparing each fixed dose combination Arm to the reference Arm (Arm 1) is also reported for each PK parameter.

TABLE 9
PK Parameters of Compound (III)

| mean ± | | | | |
|---|---|---|---|---|
| SD, | | | | |
| tₘₐₓ: | Arm 1: Single | | | |
| median | agents | | | |
| [range] | (reference) | Arm 2 | Arm 3 | Arm 4 |
| N | 18^{a} | 18^{a} | 18^{b} | 17^{c} |
| Cₘₐₓ, | 484 ± 177 | 544 ± 225 | 617 ± 295 | 745 ± 381 |
| ng/mL | | | | |
| tₘₐₓ, h | 2.00 (1.00-4.00) | 2.00 (0.50-4.00) | 2.00 (1.00-4.00) | 2.00 (1.00-4.00) |
| AUCₗₐₛₜ, | 1156 ± 497 | 1258 ± 411 | 1436 ± 450 | 1834 ± 866 |
| ng · h/mL | | | | |
| AUC_{∞}, | 1183 ± 524 | 1299 ± 420 | 1576 ± 434 | 1793 ± 889 |
| ng · h/mL | | | | |
| t_{1/2term}, h | 0.6 ± 0.1 | 0.7 ± 0.2 | 0.7 ± 0.1 | 0.6 ± 0.1 |

Least Square Means Ratio (90% CI), %

| | | Arm 2 vs | Arm 3 vs | Arm 4 vs |
|---|---|---|---|---|
| | | reference | reference | reference |
| N | — | 18^{a} vs 18^{a} | 18^{b} vs 18^{a} | 17^{c} vs 18^{a} |
| Cₘₐₓ | — | 109.68 | 122.40 | 145.56 |
| | | (84.80-141.86) | (94.63-158.31) | (112.12-188.97) |
| AUCₗₐₛₜ | — | 110.22 | 127.06 | 151.72 |
| | | (88.85-136.72) | (102.43-157.61) | (121.93-188.80) |
| AUC_{∞} | — | 111.86 | 138.97 | 144.80 |
| | | (88.52-141.34) | (108.55-177.92) | (114.15-183.67) |

^{a}N = 16 for AUC_{∞} and t_{1/2term}
^{b}N = 13 for AUC_{∞} and t_{1/2term}
^{c}N = 15 for AUC_{∞} and t_{1/2term}

The plasma concentration of SMV over 12 hours was measured for each Arm of the study. The results are shown in FIG. 10. SMV plasma concentration on the y-axis is measured in ng/mL and time is measured in hours on the x-axis. SMV, ODV, and Compound (III) were administered as a single agent in the combination therapy (Arm 1) or a fixed dose combination (FDC) (Arm 2, Arm 3, and Arm 4). The curve with the open circles is the concentration curve obtained when ODV, SMV, and Compound III are administered together as single agents (Arm 1). The curve with the closed circles is the concentration curve when ODV, SMV, and Compound III are administered as a FDC: SMV 100 mg+ODV 50 mg as a spray dried solid dispersion with HPMC-AS L in a ratio ODV/HPMC-AS L 1/1 w/w+Compound III 800 mg (Arm 2). The curve with the open squares is the concentration curve when ODV, SMV, and Compound III are administered as a FDC: SMV 100 mg+ODV 50 mg as a spray dried solid dispersion with HPMC-AS L in a ratio ODV/HPMC-AS L 1/3 w/w+Compound III 800 mg (Arm 3). The curve with the closed squares is the concentration curve when ODV, SMV, and Compound III are administered as a FDC: SMV 100 mg+ODV 50 mg as a spray dried solid dispersion with copovidone and poloxamers+Compound III 800 mg (Arm 4).

Table 11 reports the Cₘₐₓ, tₘₐₓ, AUCₗₐₛₜ, AUC_{∞}, and t_{1/2term} for SMV for each Arm of the study. The least square means ratio comparing each fixed dose combination Arm to the reference Arm (Arm 1) is also reported for each PK parameter.

TABLE 10
PK Parameters of SMV

| mean ± | | | | |
|---|---|---|---|---|
| SD, tₘₐₓ: | Arm 1: Single | | | |
| median | agents | | | |
| [range] | (reference) | Arm 2 | Arm 3 | Arm 4 |
| N | 18 | 18 | 18 | 18 |
| Cₘₐₓ, | 777 ± 225 | 942 ± 431 | 906 ± 321 | 856 ± 318 |
| ng/mL | | | | |
| tₘₐₓ, h | 6.00 (3.00-8.00) | 6.00 (4.00-12.00) | 6.00 (4.00-12.00) | 6.00 (3.00-10.00) |
| AUCₗₐₛₜ, | 9418 ± 3376 | 12704 ± 6260 | 12893 ± 5828 | 11634 ± 6295 |
| ng · h/mL | | | | |
| AUC_{∞}, | 9922 ± 3432 | 13228 ± 6433 | 13449 ± 6015 | 12087 ± 6414 |
| ng · h/mL | | | | |
| t_{1/2term}, h | 8.3 ± 1.8 | 8.7 ± 2.3 | 9.1 ± 1.6 | 8.5 ± 1.5 |

Least Square Means Ratio (90% CI), %

| | | Arm 2 vs | Arm 3 vs | Arm 4 vs |
|---|---|---|---|---|
| | | reference | reference | reference |
| N | — | 18 vs 18 | 18 vs 18 | 18 vs 18 |
| Cₘₐₓ | — | 116.50 | 115.24 | 108.22 |
| | | (97.47-139.25) | (96.41-137.74) | (90.54-129.35) |
| AUCₗₐₛₜ | — | 127.79 | 133.85 | 117.50 |
| | | (101.26-161.28) | (106.06-168.92) | (93.11-148.29) |
| AUC_{∞} | — | 126.19 | 132.28 | 116.00 |
| | | (100.48-158.47) | (105.33-166.12) | (92.37-145.68) |

The plasma concentration of ODV over 12 hours was measured for each arm of the study. The results are shown in FIG. 10. The ODV plasma concentration on the y-axis is measured in ng/mL and time is measured in hours on the x-axis. SMV, ODV, and Compound (III) were administered as a single agent in the combination therapy (Arm 1) or a fixed dose combination (FDC) (Arm 2, Arm 3, and Arm 4). The curve with the open circles is the concentration curve obtained when ODV, SMV, and Compound III are administered together as single agents (Arm 1). The curve with the closed circles is the concentration curve when ODV, SMV, and Compound III are administered as a FDC: SMV 100 mg+ODV 50 mg as a spray dried solid dispersion with HPMC-AS L in a ratio ODV/HPMC-AS L 1/1 w/w+Compound III 800 mg (Arm 2). The curve with the open squares is the concentration curve when ODV, SMV, and Compound III are administered as a FDC: SMV 100 mg+ODV 50 mg as a spray dried solid dispersion with HPMC-AS L in a ratio ODV/HPMC-AS L 1/3 w/w+Compound III 800 mg (Arm 3). The curve with the closed squares is the concentration curve when ODV, SMV, and Compound III are administered as a FDC: SMV 100 mg+ODV 50 mg as a spray dried solid dispersion with copovidone and poloxamers+Compound III 800 mg (Arm 4).

Table 11 reports the Cₘₐₓ, tₘₐₓ, AUCₗₐₛₜ, AUC_{∞}, and t_{1/2term} for ODV for each Arm of the study. The least square means ratio comparing each fixed dose combination Arm to the reference Arm (Arm 1) is also reported for each PK parameter.

TABLE 11
PK Parameters of ODV

| mean ± | | | | |
|---|---|---|---|---|
| SD, tₘₐₓ: | Arm 1: Single | | | |
| median | agents | | | |
| [range] | (reference) | Arm 2 | Arm 3 | Arm 4 |
| N | 18^{a} | 18^{b} | 18^{c} | 18^{b} |
| Cₘₐₓ, | 186 ± 60.4 | 151 ± 54.0 | 301 ± 80.5 | 298 ± 81.7 |
| ng/mL | | | | |
| tₘₐₓ, h | 6.00 (6.00-12.00) | 6.00 (6.00-12.00) | 6.00 (6.00-12.00) | 6.00 (3.00-12.00) |
| AUCₗₐₛₜ, | 6323 ± 2323 | 5592 ± 1889 | 11488 ± 4387 | 11127 ± 3354 |
| ng · h/mL | | | | |
| AUC_{∞}, | 7076 ± 2144 | 7040 ± 2130 | 14818 ± 5122 | 15717 ± 3555 |
| ng · h/mL | | | | |
| t_{1/2term}, h | 168.6 ± 21.7 | 169.0 ± 24.5 | 176.0 ± 29.5 | 167.8 ± 22.4 |

Least Square Means Ratio (90% CI), %

| | | Arm 2 vs | Arm 3 vs | Arm 4 vs |
|---|---|---|---|---|
| | | reference | reference | reference |
| N | — | 18 vs 18 | 18 vs 18 | 18 vs 18 |
| Cₘₐₓ | — | 81.00 | 165.30 | 163.29 |
| | | (67.57-97.10) | (137.89-198.16) | (136.21-195.75) |
| AUCₗₐₛₜ | — | 89.66 | 182.32 | 180.14 |
| | | (73.71-109.05) | (149.90-221.76) | (148.10-219.12) |

^{a}N = 7 for AUC_{∞} and N = 17 for f_{1/2term}
^{b}N = 9 for AUC_{∞}
^{c}N = 8 for AUC_{∞} and N = 17 for f_{1/2term}

The plasma concentration of Compound A-1 over 12 hours was measured for each Arm of the study. The results are shown in FIG. 12. Compound A-1 plasma concentration on the y-axis is measured in ng/mL and time is measured in hours on the x-axis, SMV, ODV, and Compound (III) were administered as a single agent in the combination therapy (Arm 1) or a fixed dose combination (FDC) (Arm 2, Arm 3, and Arm 4). The curve with the open circles is the concentration curve obtained when ODV, SMV, and Compound III are administered together as single agents (Arm 1). The curve with the closed circles is the concentration curve when ODV, SMV, and Compound III are administered as a FDC: SMV 100 mg+ODV 50 mg as a spray dried solid dispersion with HPMC-AS L in a ratio ODV/HPMC-AS L 1/1 w/w+Compound III 800 mg (Arm 2). The curve with the open squares is the concentration curve when ODV, SMV, and Compound III are administered as a FDC: SMV 100 mg+ODV 50 mg as a spray dried solid dispersion with HPMC-AS L in a ratio ODV/HPMC-AS L 1/3 w/w+Compound III 800 mg (Arm 3). The curve with the closed squares is the concentration curve when ODV, SMV, and Compound III are administered as a FDC: SMV 100 mg+ODV 50 mg as a spray dried solid dispersion with copovidone and poloxamers+Compound III 800 mg (Arm 4).

Table 12 reports the Cₘₐₓ, tₘₐₓ, AUCₗₐₛₜ, AUC_{∞}, and t_{1/2term} for Compound A-1 for each Arm of the study. The least square means ratio comparing each fixed dose combination Arm to the reference Arm (Arm 1) is also reported for each PK parameter.

TABLE 12
PK Parameters of Compound A-1

| mean ± | | | | |
|---|---|---|---|---|
| SD, tₘₐₓ: | Arm 1: Single | | | |
| median | agents | | | |
| [range] | (reference) | Arm 2 | Arm 3 | Arm 4 |
| N | 18^{a} | 18^{b} | 18^{c} | 17^{d} |
| Cₘₐₓ, | 498 ± 154 | 681 ± 268 | 598 ± 192 | 607 ± 208 |
| ng/mL | | | | |
| tₘₐₓ, h | 4.00 (3.00-6.00) | 4.00 (3.00-8.00) | 4.00 (3.00-6.00) | 4.00 (2.00-6.00) |
| AUCₗₐₛₜ, | 3912 ± 1153 | 4684 ± 1547 | 4714 ± 1310 | 4693 ± 1418 |
| ng · h/mL | | | | |
| AUC_{∞}, | 4532 ± 1797 | 4369 ± 1345 | 5384 ± 1564 | 4555 ± 787 |
| ng · h/mL | | | | |
| t_{1/2term}, h | 19.6 ± 2.7 | 20.5 ± 3.3 | 20.2 ± 3.5 | 18.3 ± 3.5 |

Least Square Means Ratio (90% CI), %

| | | Arm 2 vs | Arm 3 vs | Arm 4 vs |
|---|---|---|---|---|
| | | reference | reference | reference |
| N | — | 18 vs 18 | 18 vs 18 | 18 vs 18 |
| Cₘₐₓ | — | 133.00 | 119.83 | 121.37 |
| | | (110.01-160.79) | (99.12-144.87) | (100.11-147.13) |
| AUCₗₐₛₜ | — | 118.54 | 121.29 | 120.45 |
| | | (100.56-139.73) | (102.89-142.98) | (101.94-142.33) |

^{a}N = 6 for AUC_{∞} and t_{1/2term}
^{b}N = 8 for AUC_{∞} and t_{1/2term}
^{c}N = 5 for AUC_{∞} and t_{1/2term}
^{d}N = 8 for AUC_{∞} and N = 9 for t_{1/2term}

The plasma concentration of Compound A-3 over 12 hours was measured for each Arm of the study. The results are provided in FIG. 13. Compound A-3 plasma concentration on the y-axis is measured in ng/mL and time is measured in hours on the x-axis. SMV, ODV, and Compound (III) were administered as a single agent in the combination therapy (Arm 1) or a fixed dose combination (FDC) (Arm 2, Arm 3, and Arm 4). The curve with the open circles is the concentration curve obtained when ODV, SMV, and Compound III are administered together as single agents (Arm 1). The curve with the closed circles is the concentration curve when ODV, SMV, and Compound III are administered as a FDC: SMV 100 mg+ODV 50 mg as a spray dried solid dispersion with HPMC-AS L in a ratio ODV/HPMC-AS L 1/1 w/w+Compound III 800 mg (Arm 2). The curve with the open squares is the concentration curve when ODV, SMV, and Compound III are administered as a FDC: SMV 100 mg+ODV 50 mg as a spray dried solid dispersion with HPMC-AS L in a ratio ODV/HPMC-AS L 1/3 w/w+Compound III 800 mg (Arm 3). The curve with the closed squares is the concentration curve when ODV, SMV, and Compound III are administered as a FDC: SMV 100 mg+ODV 50 mg as a spray dried solid dispersion with copovidone and poloxamers+Compound III 800 mg (Arm 4).

Table 13 reports the Cₘₐₓ, tₘₐₓ, AUCₗₐₛₜ, AUC_{∞}, and t_{1/2term} for Compound A-3 for each Arm of the study. The least square means ratio comparing each fixed dose combination Arm to the reference Arm (Arm 1) is also reported for each PK parameter.

TABLE 13
PK Parameters of Compound A-3

| mean ± | | | | |
|---|---|---|---|---|
| SD, tₘₐₓ: | Arm 1: Single | | | |
| median | agents | | | |
| [range] | (reference) | Arm 2 | Arm 3 | Arm 4 |
| N | 18 | 18^{a} | 18 | 17^{b} |
| Cₘₐₓ, | 163 ± 61.8 | 163 ± 45.2 | 189 ± 49.7 | 197 ± 62.1 |
| ng/mL | | | | |
| tₘₐₓ, h | 3.00 (2.00-4.00) | 3.00 (2.00-4.00) | 3.50 (2.00-6.00) | 4.00 (2.00-6.00) |
| AUCₗₐₛₜ, | 800 ± 307 | 790 ± 157 | 895 ± 223 | 909 ± 244 |
| ng · h/mL | | | | |
| AUC_{∞}, | 823 ± 311 | 817 ± 133 | 914 ± 226 | 926 ± 245 |
| ng · h/mL | | | | |
| t_{1/2term}, h | 2.6 ± 1.4 | 2.3 ± 0.6 | 2.9 ± 1.5 | 2.5 ± 0.7 |

Least Square Means Ratio (90% CI), %

| | | Arm 2 vs | Arm 3 vs | Arm 4 vs |
|---|---|---|---|---|
| | | reference | reference | reference |
| N | — | 18^{a} vs 18 | 18 vs 18 | 17 vs 18 |
| Cₘₐₓ | — | 102.81 | 119.82 | 123.77 |
| | | (86.27-122.52) | (100.54-142.78) | (103.60-147.88) |
| AUCₗₐₛₜ | — | 103.24 | 115.78 | 116.82 |
| | | (88.23-120.80) | (98.94-135.48) | (99.61-137.01) |
| AUC_{∞} | — | 104.40 | 114.92 | 115.62 |
| | | (89.17-122.25) | (98.61-133.93) | (98.99-135.05) |

^{a}N = 16 for AUC_{∞} and f_{1/2term}
^{b}N = 18 for t_{1/2term}

### Example 14. Examples of Embodiments

In one aspect, the invention is the use of a combination for the treatment of hepatitis C virus infection comprising three direct acting antivirals (DAAs), a HCV NS3/4A serine protease inhibitor, a HCV NS5A inhibitor and a NS5B polymerase inhibitor (a nucleoside or non-nucleoside), in a treatment regime of 4-12 weeks (for example 4-6 weeks or 6-12 weeks). Such 3DAA combinations may refer to Simeprevir, ODV and a Compound of formula (III).

In particular, the present disclosure is directed to methods of treating HCV in a patient comprising administering to the patient an effective amount of: a HCV NS3/4A serine protease inhibitor; a HCV NS5A inhibitor; and a NS5B polymerase inhibitor; wherein the administration terminates after a period of time that is 6 weeks or less (e.g. 6 weeks or, in some embodiments, 5 or 4 weeks). In an alternative embodiment, the administration terminates after a period of time that is 12 weeks or less (e.g. 12 weeks or 8 weeks). More specifically, the present disclosure is directed to methods of treating HCV in a patient comprising administering to the patient an effective amount of: Simeprevir, or a pharmaceutically acceptable salt thereof; Odalasvir, or a pharmaceutically acceptable salt thereof, and Compound of formula (III), or a pharmaceutically acceptable salt thereof; wherein the administration terminates after a period of time that is 6 weeks or less (e.g. 6 weeks or, in some embodiments, 5 or 4 weeks). In an alternative embodiment, the administration terminates after a period of time that is 12 weeks or less (e.g. 12 weeks or 8 weeks). The invention also includes the specific combination as such comprising: (i) a compound of formula (I), or a pharmaceutically-acceptable salt thereof, (ii) a compound of formula (II), or a pharmaceutically-acceptable salt thereof; and (iii) a compound of formula (III), or a pharmaceutically-acceptable salt thereof. For instance, compound of formula (I) may be in the form of an amorphous sodium salt, the compound of formula (II) may be a crystalline form that in some embodiments is not in the form of a salt, and the compound of formula (III) may be an anhydrous crystalline form that in some embodiments is not in the form of a salt or solvate.

The present disclosure provides methods of treating HCV in a patient comprising administering to the patient an effective amount of: a HCV NS3/4A serine protease inhibitor; a HCV NS5A inhibitor; and a NS5B polymerase inhibitor; wherein the administration terminates after a period of time that is 6 weeks or less (e.g. 6 weeks or, in some embodiments, 5 or 4 weeks.

More specifically, the present disclosure provides methods of treating HCV in a patient comprising administering to the patient an effective amount of a compound of formula (I) (Simeprevir) or a pharmaceutically acceptable salt thereof, a compound of formula (II) (“Odalasvir”): or a pharmaceutically acceptable salt thereof, and a compound of formula (III) (also referred to as Compound (III) or “Cpd (III)”) or a pharmaceutically acceptable salt thereof, wherein said administration terminates after a period of time that is 6 weeks or less (e.g. 6 weeks or, in some embodiments, 5 or 4 weeks). In another embodiment, the administration period may also be a period of anything between 4 and 12 weeks (e.g. 4, 6, 8 or 12 weeks). Patients who can be treated using the described methods are in some embodiments human. Other warm-blooded animals can also be treated.

In an alternative embodiment of the invention, there is provided a specific combination of: (i) a compound of formula (I), or a pharmaceutically-acceptable salt thereof, (ii) a compound of formula (II), or a pharmaceutically-acceptable salt thereof; and (iii) a compound of formula (III), or a pharmaceutically-acceptable salt thereof. In some embodiments, the pharmaceutically acceptable salt of compound of formula (I) is a sodium salt, for example the monosodium salt. In some embodiments, compound of formula (II) is in a crystalline non-salt form. In some embodiments, compound of formula (III) is in an anhydrous crystalline non-salt form, which still in other embodiments is in the form of an anhydrous crystalline form that is neither a salt nor solvate. Embodiments of this invention showed that administration of compounds of formulas (I)-(III) or pharmaceutically acceptable salts thereof unexpectedly led to advantageous influences amongst them as manifested by PK analysis of the same, which could lead to a reduction in the treatment time and/or effective treatment dosages in comparison with those that would be envisaged according to conventional treatment methods and compounds.

The present disclosure is also directed to a combination comprising Simeprevir (a compound of formula (I)), or a pharmaceutically acceptable salt thereof; Odalasvir (a compound of formula (II)), or a pharmaceutically acceptable salt thereof; and a compound of formula (III), or a pharmaceutically acceptable salt thereof, for use in an HCV treatment regime that terminates after a period of time that is 6 weeks or less, for example, 6, 5, or 4 weeks. In an alternative embodiment, such a treatment regime may terminate after a period of time that is 6 to 12 weeks (e.g. 6 weeks, 8 weeks or 12 weeks). In some embodiments, the administration of the compounds of formulas (I), (II), and (III), or any salt form(s) thereof, terminates after a period of time that is less than 6 weeks, for example, 5, or 4 weeks. In other embodiments, the administration terminates after a period of time that is 4 weeks.

In some embodiments, the patients treated according to the described methods include the following patient categories: —all genotypes; —treatment naïve; —treatment-experienced; —compensated liver patients; —decompensated liver patients; —cirrhotics; —non-cirrhotics; —patients with fibrosis (e.g. high levels of fibrosis); —all ethnicities; —co-infected (particularly co-infected with HIV); —liver transplant patients; —patients with polymorphisms (e.g. Q80K, etc); —all IL28 status patients. HCV infections that can be treated according to the disclosed methods include HCV genotype 1 infections, for example, HCV genotype 1a infections. Other infections that can be treated using the disclosed methods include HCV genotype 4 infections. However, in an embodiment, the methods disclosed treat any HCV genotype (“pan-genotypic treatment”). HCV genotyping can be performed using methods known in the art, for example, VERSANT™ HCV Genotype 2.0 Assay Line Probe Assay (LiPA).

In diverse embodiments of methods according to this invention, compounds of formulas (I), (II), and (III), or pharmaceutically acceptable salts thereof are administered once per day during the period of administration. In some embodiments, they can be co-administered, in others sequentially administered, while in still others they can be administered substantially simultaneously. In some of the latter embodiments, administration entails taking such compounds or pharmaceutically acceptable salts thereof within 30 minutes or less of each other, in some embodiments 15 minutes or less of each other. In some embodiments, the compounds of formulas (I), (II), and (III), or pharmaceutically acceptable salts thereof are administered once per day, at approximately the same time each day. For example, the compounds of formulas (I), (II), and (III), or pharmaceutically acceptable salts thereof are administered within a time range of 4 hours of the original time of administration on the first day, that is, ±2 hours, or ±1 hour, or in still other embodiments±30 minutes of the time on the original administration day. In some embodiments, the compounds of formulas (I), (II), and (III), or pharmaceutically acceptable salts thereof are administered as separate oral capsules or oral tablets. Other formulations, e.g., for the compound of formula (II), may include solid dispersions. The combination of compounds as described herein may be co-administered, sequentially administered, or administered substantially simultaneously.

### Example 15. Phase 2b Study Design

A Phase 2b, multicenter, randomized, open-label study is carried out to investigate the efficacy, safety and pharmacokinetics of a 8-, 6- or 4-week (e.g. 8- or 6-week) treatment regimen with Simeprevir, Odalasvir and Compound (III), followed by a 24-week post-treatment follow-up, in treatment-naïve and treatment experienced subjects with chronic hepatitis C virus Genotype 1, 2, 3, 4, 5 and 6 infection, with and without cirrhosis. This Phase 2b is a multicenter study that includes a screening period of 6 weeks, a treatment period of 6 or 8 or 12-weeks (and the 24-weeks post-treatment follow-up period) and can be extended with an additional 4 weeks. The total study duration for each subject is approximately 36 to 42 weeks. This study can be used to confirm the activity of the three direct-acting antiviral agent (DAA) combination of Simeprevir (SMV) (HCV NS3A4 protease inhibitor), Odalasvir (ODV) (a second generation HCV NS5A inhibitor) and Compound (III) (HCV NS5B inhibitor) and 2 DAA combination of ODV and Compound (III) directed at 3 different targets in the HCV life cycle.

TABLE 14
The Arms and the Assigned Interventions of the Phase 2b Study Design

| Arms | Assigned Interventions |
|---|---|
| Experimental: Cohort 1: Group A | Drug: Simeprevir |
| Participants with or without cirrhosis receive | Simeprevir 75 mg administered once daily for |
| Simeprevir 75 milligram (mg) once daily, | 6 or 8 weeks. |
| Odalasvir 50 mg once daily every other day | Drug: Odalasvir |
| and Compound (III) 800 mg or 1200 mg once | Odalasvir 50 mg administered once daily |
| daily for 6 weeks. | every other day for 6 or 8 or 12 weeks. |
| | Drug: Compound (III) |
| | Compound (III) 800 mg or 1200 mg |
| | administered as once daily for 6 or 8 or 12 |
| | weeks. |
| Experimental: Cohort 1: Group B | Drug: Simeprevir |
| Participants with or without cirrhosis receive | Simeprevir 75 mg administered once daily for |
| Simeprevir 75 mg once daily, Odalasvir 50 mg | 6 or 8 weeks. |
| once daily every other day and Compound | Drug: Odalasvir |
| (III) 800 mg or 1200 mg once daily for 8 | Odalasvir 50 mg administered once daily |
| weeks. | every other day for 6 or 8 or 12 weeks. |
| | Drug: Compound (III) |
| | Compound (III) 800 mg or 1200 mg |
| | administered as once daily for 6 or 8 or 12 |
| | weeks. |
| Experimental: Cohort 2: Group C | Drug: Odalasvir |
| Participants with and without cirrhosis receive | Odalasvir 50 mg administered once daily |
| Compound (III) 800 mg or 1200 mg | every other day for 6 or 8 or 12 weeks |
| and Odalasvir 50 mg once daily every | Drug: Compound (III) |
| other day for 8 weeks. | Compound (III) 800 mg or 1200 mg |
| | administered as once daily for 6 or 8 or 12 |
| | weeks. |
| Experimental: Cohort 2: Group D | Drug: Odalasvir |
| Participants with and without cirrhosis receive | Odalasvir 50 mg administered once daily |
| Compound (III) 800 mg or 1200 mg | every other day for 6 or 8 or 12 weeks. |
| and Odalasvir 50 mg once daily every | Drug: Compound (III) |
| other day for 12 weeks. | Compound (III) 800 mg or 1200 mg |
| | administered as once daily for 6 or 8 or 12 |
| | weeks. |

This specification has been described with reference to embodiments of the invention. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

What is claimed:

1. A composition comprising Compound (I) (Simeprevir), Compound (II) (Odalasvir), and Compound (III) or independently its salt, hydrate or solvate:

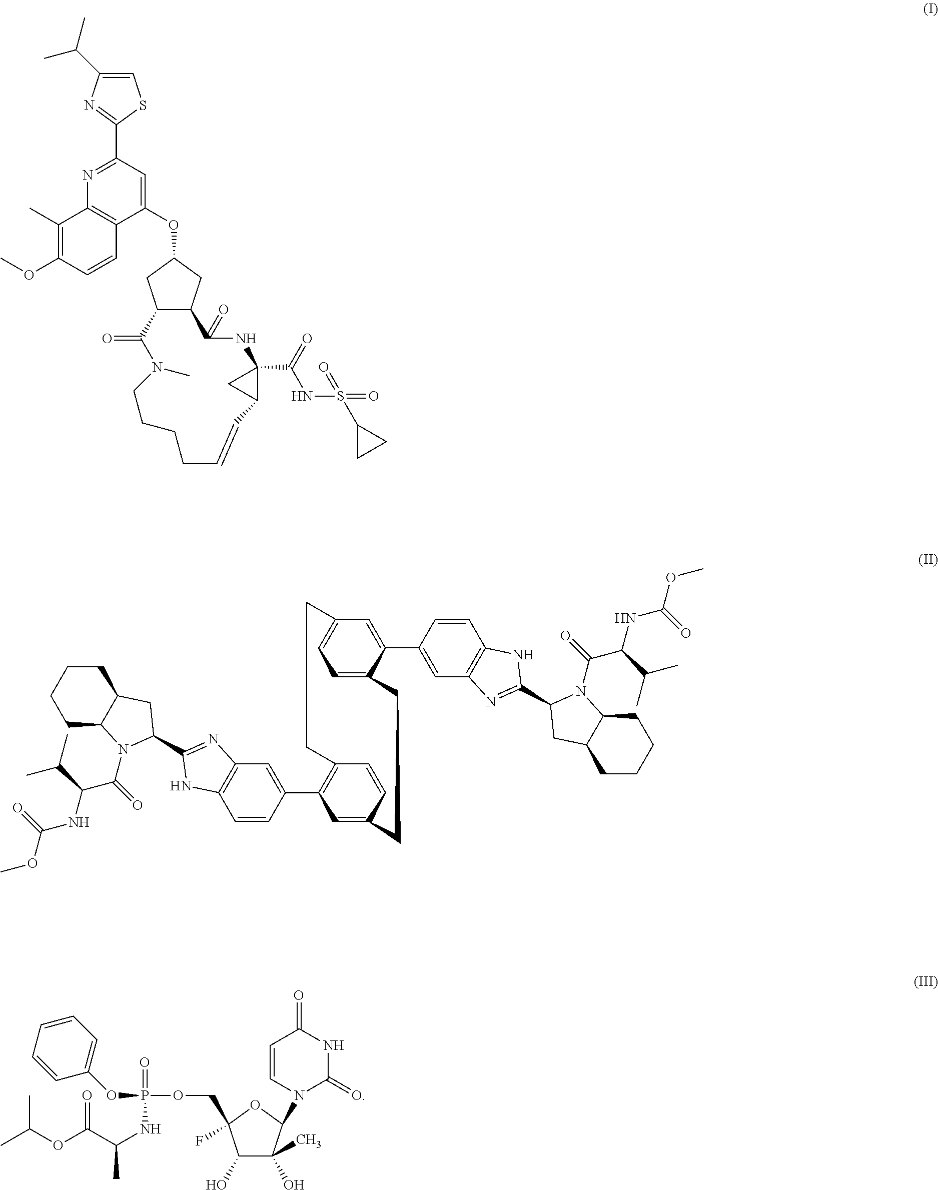

2. The composition of claim 1, wherein the composition is in a form suitable for oral delivery.

3. The composition of claim 1, further comprising a pharmaceutically acceptable excipient.

4. A method of treating HCV in a patient comprising administering to the patient a substantially simultaneous combination of an effective amount of: 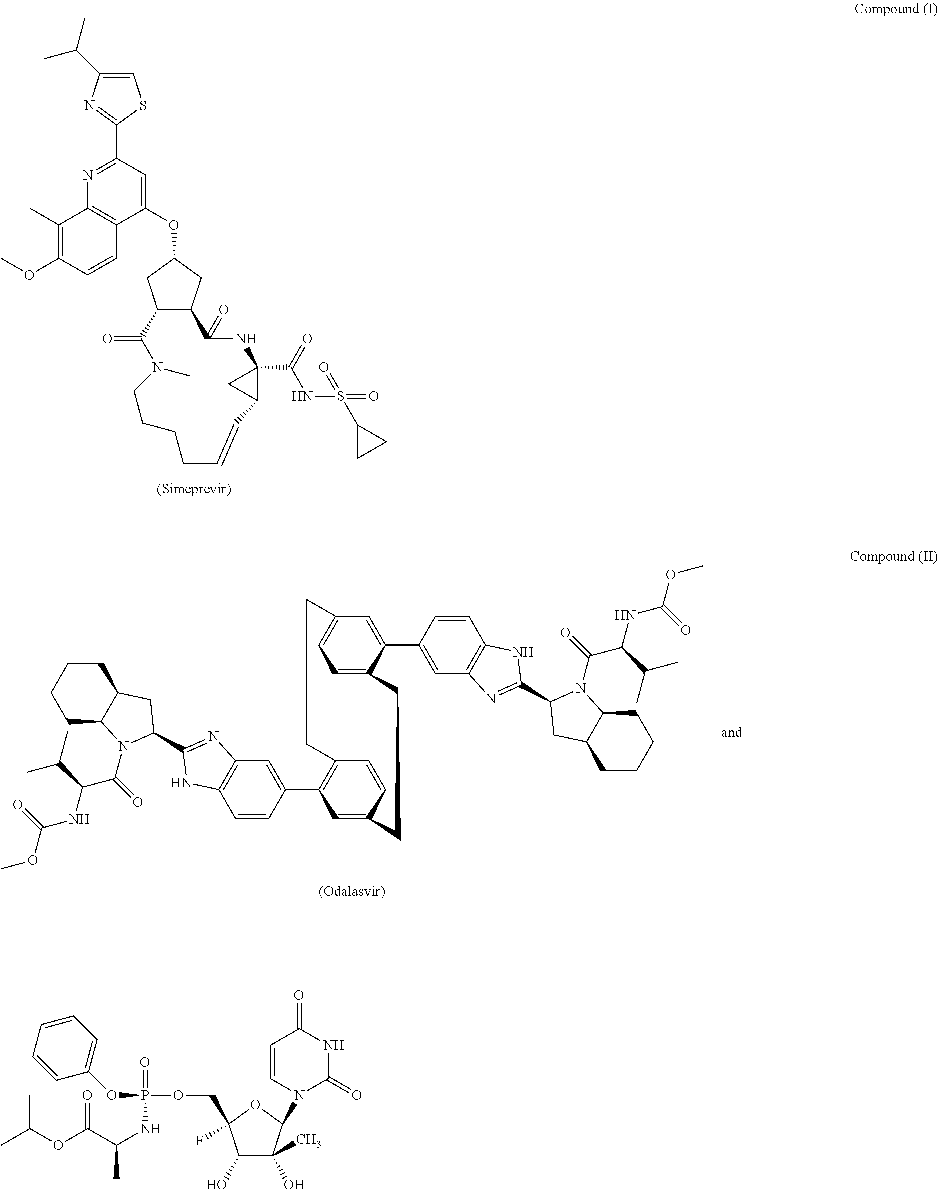
or independently a pharmaceutically acceptable salt, hydrate, or solvate thereof,
wherein the treatment period is 8 weeks or less.

5. The method of claim 4, wherein the treatment period is 7 weeks or less.

6. The method of claim 4, wherein the treatment period is 6 weeks or less.

7. The method of claim 4, wherein the treatment period is 5 weeks or less.

8. The method of claim 4, wherein the treatment period is 4 weeks or less.

9. The method of claim 4 that does not include administering interferon, PEGylated interferon, or ribavirin to the patient.

10. The method of claim 4, wherein the patient is a treatment naïve patient.

11. The method of claim 4, wherein the patient is a treatment experienced patient.

12. The method of claim 4, wherein the HCV comprises HCV genotype 1

13. The method of claim 4, wherein the HCV comprises HCV genotype 2.

14. The method of claim 4, wherein the HCV comprises HCV genotype 3.

15. The method of claim 4, wherein the HCV comprises HCV genotype 4.

16. The method of claim 4, wherein the HCV comprises HCV genotype 5.

17. The method of claim 4, wherein the HCV comprises HCV genotype 6.

18. The method of claim 4, wherein the drug combination exhibits pan-genotypic efficacy.

19. The method of claim 4, wherein the patient achieves sustained virologic response with an HCV RNA level of less than LLOQ for up to 24 weeks after termination of treatment.

20. The method of claim 4, wherein the patient achieves sustained virologic response with an HCV RNA level of less than LLOQ for up to 12 weeks after termination of treatment.

21. The method of claim 4, wherein the patient achieves sustained virologic response with an HCV RNA level of less than LLOQ for up to 4 weeks after termination of treatment.

22. The method of claim 4, wherein the compounds, or salts thereof, are each administered once per day during the period of administration.

23. The method of claim 4, wherein the compounds, or salts thereof, are administered in one fixed dosage form.

24. The method of claim 4, wherein Compound (I), or a pharmaceutically acceptable salt thereof, is administered in an amount that is about 100 mg per day.

25. The method of claim 4, wherein Compound (II), or a pharmaceutically acceptable salt thereof, is administered in an amount that is about 10 to 25 mg per day.

26. The method of claim 4, wherein Compound (III), or a pharmaceutically acceptable salt thereof, is administered in an amount that is 800 mg per day.

27. A pharmaceutical fixed dosage form of the combination of the compounds: 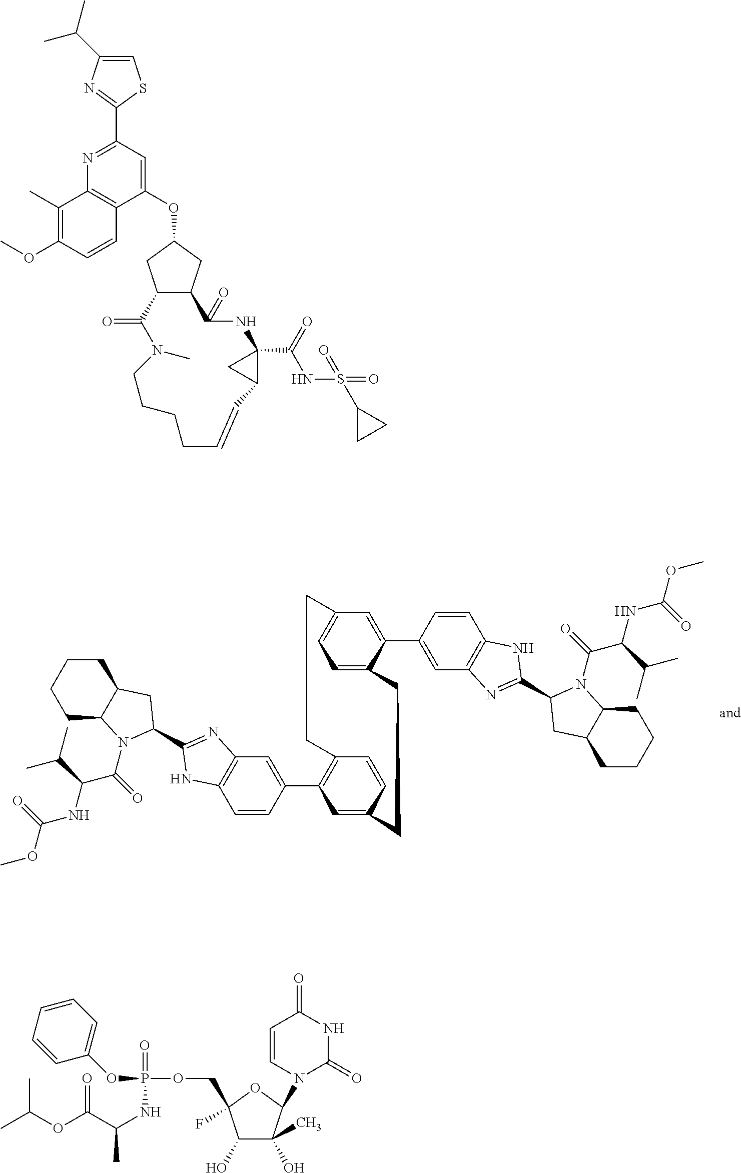
or independently a pharmaceutically acceptable salt, solvate or hydrate thereof.

28. The fixed dosage combination of claim 27 which comprises a spray dried dispersion.

29. The fixed dosage combination as claimed in claim 27, which comprises a granulo layered solid dispersion.

30. A package for providing a therapeutic dosage regime for administration to a patient infected with HCV that includes: Compound (I), Compound (II), and Compound (III), or pharmaceutically acceptable salts thereof, wherein the compounds may be in individual dosage forms or two or more are in combined dosage forms, and wherein the Compounds are as defined in claim 1.

31. The package of claim 30, wherein Compound I is provided in a dosage of 75 or 100 mg optionally as its sodium salt, Compound II is provided in a dosage of 10 to 25 mg and Compound III is provided in a dosage of 800 mg.

32. The package of claim 30, wherein the dosages are solid dosage forms.

33. The composition of claim 1, wherein Compound (I) is in the form of a sodium salt and Compound (II) and Compound (III) are not in the form of a salt.

34. The composition of claim 1, wherein Compound (II) is in the form of a sodium salt and Compound (I) and Compound (III) are not in the form of a salt.

35. The composition of claim 1, wherein Compound (I) is present in the composition as a spray dried dispersion.

36. The composition of claim 1, wherein Compound (II) is present in the composition as a spray dried dispersion.

37. The method of claim 4, wherein the patient is non-cirrhotic.

38. The method of claim 4, wherein the patient is a human.
